(19) United States
(12) Patent Application Publication
(10) Pub. No.: US 2006/0024278 A1
(43) Pub. Date: Feb. 2, 2006

(54) Methods and products related to the production of inner ear hair cells

(75) Inventor: Zheng-Yi Chen, Somerville, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: The General Hospital Corporation, Boston, MA (US)

(21) Appl. No.: 11/041,907

(22) Filed: Jan. 24, 2005

Related U.S. Application Data
(60) Provisional application No. 60/538,917, filed on Jan. 23, 2004.

(51) Int. Cl. A61K 48/00

(52) U.S. Cl. 424/93.21; 514/44

(57) ABSTRACT

The invention relates to the generation of inner ear sensory epithelial cells through the manipulation of the expression level or function of the genes and/or proteins involved in the retinoblastoma (Rb) pathway, particularly retinoblastoma family members, such as Rb1. Methods for generating inner ear sensory epithelial cells and for restoring hearing or balance in a subject, therefore, are provided. The invention further relates to cell lines of inner ear sensory epithelial cells, such as progenitor, supporting or hair cells, where the expression level or function of the retinoblastoma genes and/or retinoblastoma proteins has been manipulated. In addition to these methods, compositions of agents for use in or the cells produced by such methods provided are also included. Finally, the invention also relates to methods and compositions for the generation of inner ear sensory epithelial cells through the manipulation of Isl-1 alone or in combination with the manipulation of retinoblastoma gene and/or retinoblastoma protein.

### RELATED APPLICATIONS

This application claims priority under 35 U.S.C. §119 from U.S. provisional application serial No. 60/538,917, filed Jan. 23, 2004, the entire contents of which is herein incorporated by reference.

### GOVERNMENT SUPPORT

Aspects of the invention may have been made using funding from National Institute on Deafness and Other Communication Disorders Grant DC04546. Accordingly, the Government may have rights in the invention.

### FIELD OF THE INVENTION

The invention relates to the generation of hair cells of the ear through the control of the genes involved in the retinoblastoma pathway, particularly Rb family proteins, and/or their protein expression products, related methods and compositions.

### BACKGROUND OF THE INVENTION

Inner ear hair cells play a crucial role in hearing and balance. The essential function and delicate structure of the hair cells make them particularly prone to damage. Millions of people suffer from permanent hearing impairment as the result of hair cell loss (Corwin, 1998).

Both environmental and genetic factors are involved in hair cell death. For instance, otoxic drugs, noise, as well as mutations in many genes can cause hair cell death (Schacht, 1999; Wu, 2002; Morton, 2002; Duan, 2002). For example, genes involved in the function of hair cells, such as myosin 15, can cause deafness when mutated (Self et al., 1998; Wang et al., 1998). Noise and viral infection are other leading causes of hair cell death (Duan et al., 2002; Morton, 2002). Moreover, like the majority of neurons, mammalian hair cells in general do not undergo spontaneous regeneration after damage, further complicating the process of functional recovery of hearing. Therefore, any therapy aimed at regenerating hair cells will require an understanding of hair cell development.

Hair cell development involves permanent exit of the cell cycle by the progenitor cells and the initiation of fate determination and terminal differentiation of the hair cell. A bHLH transcription factor, Math1, has been identified as the gene controlling hair cell fate, and expression of Math1 can trigger downstream hair cell terminal differentiation in the supporting cells, although the mechanism involved is not known (Bermingham et al., 1999; Kawamoto et al., 2003; Zheng and Gao, 2000). Other pathways, most notably, the retinoic acid and Notch pathways, are involved in the induction of hair cells either from the progenitor cell pool or from the supporting cells at early stages of development. (Bryant et al. 2002; Kiernan et al., 2001; Lanford et al., 1999; Raz and Kelley 1999; Zhang et al., 2000; Zine et al., 2000). One of the key questions in hair cell development is the identity of the genes and the pathways that regulate permanent cell cycle exit for the sensory progenitor cells, as well as the maintenance of the quiescent postmitotic hair cells and supporting cells. It is possible that perturbations of the pathways involved may force the postmitotic sensory epithelial cells to re-enter the cell cycle, an important implication for hair cell regeneration.

It has been recently demonstrated that cell cycle exit of the progenitor cell is a key event prior to the differentiation of hair cells and supporting cells. Negative cell cycle regulators have been increasingly recognized for their important roles in maintaining the postmitotic status of the hair cells. During embryonic development, both the cochlea and the vestibule are derived from the pro-sensory patch of the otocyst which forms through invagination of the otic placode (Bryant et al., 2002). The cochlear progenitor cells are in an active cell cycle that progresses through E12.2-13.5 when a zone of non-proliferating cells (ZNPC) is defined by the expression of p27kip1, a cyclin dependent kinase inhibitor (Chen et al., 2002; Chen and Segil, 1999; Lowenheim et al., 1999) that is highly expressed in the cochlear hair cell progenitor cells but completely absent in the hair cells. It has been established that the function of p27kip1 is necessary for the cell cycle exit in the supporting cells, as in p27kip1 knockout mice both the embryonic and adult supporting cells can re-enter the cell cycle. Subsequently, the expression of p27kip1 is downregulated in the cells within the ZNPC that are destined to become the hair cells, whereas its expression is maintained in the supporting cells. Supernumerary hair cells and supporting cells are generated in p27kip1 mouse cochlea, presumably by increasing the number of progenitor cells (Chen and Segil, 1999; Lowenheim et al., 1999). p27kip1 is the only negative cell cycle regulator known to be involved in early developmental control of the sensory cells in the cochlea.

Another cyclin dependent kinase inhibitor, p19ink4d, is expressed as early as E14 in the hair cells and has been implicated in the apoptotic pathway. The hair cells in the p19ink4d null mice start to re-enter the cell cycle at P5 and those cells subsequently die through apoptosis, causing progressive hearing loss in the mouse mutant (Chen et al., 2003). Therefore, in the inner ear, the function of p27kip1 is primarily involved in the postmitotic status of the supporting cells, whereas the function of p19ink4d is involved in protecting the hair cells from apoptosis. However, no gene has been identified that is required for cell cycle arrest in the fully differentiated hair cells, or that can be manipulated in the fully differentiated hair cells to lead to regeneration of healthy and functional hair cells. From the small number of hair cells being affected in the p19ink4d cochlea it is suggestive that some other negative cell cycle controllers may also be involved in the exit of the cell cycle, similar to the role of p27kip1 in the hair cells.

The cycling of proliferating cells through the different phases of the cell cycle is mainly controlled by different types of cyclin-dependent kinases (Classon and Harlow, 2002). To exit the cell cycle the expression of the cyclin-dependent kinases has to be downregulated through inhibitory functions of different types of cyclin dependent kinase inhibitors such as p27kip1, for Cdk2. In order to keep the cells, such as hair cells and a majority of neurons, in permanent cell cycle arrest, negative cell growth control genes are required. In addition, some growth factors, such as TGF-β, can be effective growth inhibitors (Hu and Zuckerman, 2000). It is, therefore, necessary to identify those cell cycle regulators, in particular the negative growth control genes, to understand how the postmitotic status of the sensory epithelial cell is maintained. Understanding the process could shed light on the possibility of re-entering the cell cycle by quiescent cells, through relieving the negative controls.

Avian, fish and chick sensory hair cells can be regenerated after being damaged by trauma (Corwin and Cotanche, 1988; Ryals and Rubel, 1988). In addition in the avian vestibular epithelium, new hair cells are generated through supporting cell division (Jorgensen and Mathiesen, 1988). The hair cells in the mammalian inner ear, however, do not undergo spontaneous regeneration despite the fact that the vestibular system retains very limited cell division capacity (Forge et al., 1993; Warchol et al., 1993). As a consequence damage to the hair cells leads to permanent hearing impairment since new hair cells cannot be formed. Irreversible damage to the hair cells is, therefore, the main cause of hearing loss and balance problems.

### SUMMARY OF THE INVENTION

Cell cycle regulators, in particular negative cell growth control genes, have been implicated in the re-entry into the cell cycle of the inner ear sensory cells. Using a functional genomics approach, the expression profiles of the developing mouse utricle were studied. To identify critical molecules involved in the cell cycle regulation in hair cell development, microarray analysis was employed. Retinoblastoma protein (pRb) family members were implicated by their distinct expression pattern in the inner ear. Using mice bearing targeted deletion of Rb1, it was demonstrated that pRb is essential for cell cycle exit and maintenance of the postmitotic status in all hair cells. Hair cells without pRb are undergoing cell division, and are differentiated and functional, indicating a pRb independent differentiation process. Identification of the pRb family members and subsequent characterization of pRb conditional knockout mice revealed a pivotal role of retinoblastoma (Rb) in the maintenance of the quiescent state of differentiated hair cells. The capacity of differentiated, pRb-null hair cells to remain in the cell cycle, therefore, leads to hair cell regeneration by regulation of the Rb pathway.

Therefore, in one aspect a method for generating or regenerating functional, differentiated inner ear hair cells is provided. In some embodiments this method is for in vivo purposes. In other embodiments this method is for in vitro purposes. In one aspect of the invention the method for generating or regenerating functional, differentiated inner ear hair cells includes the step of eliminating or reducing the expression level or function of the retinoblastoma gene and/or retinoblastoma protein (pRb) in inner ear sensory cells by an amount effective to generate functional, differentiated inner ear hair cells.

The elimination or reduction can be accomplished either directly or indirectly. It has been demonstrated that deletion of the Rb gene leads to proliferation of the cells in which the Rb gene was deleted. This can be accomplished in, for example, progenitor cells, supporting cells or hair cells. It was found, for example, that postnatal mature hair cells proliferated after the acute elimination of the Rb1 gene. In addition, the deletion of Rb1 gave rise to proliferation of sensory progenitor cells.

Proliferation can, however, be accomplished by indirect means. For instance the elimination or reduction of Rb expression or function can be performed in one type of sensory epithelial cell in order to lead to the proliferation of another type of inner ear sensory epithelial cell. For example, it was found that supporting cells can be induced into the cell cycle when hair cells are cycling. Therefore, Rb expression or function can be reduced or eliminated, for example, in one type of sensory cell (e.g., hair cells) to result in the regeneration of another type of cell (e.g., supporting cell). Although not wishing to be bound by any theory, it is thought that this is the result of the action of signaling molecules of the cells with reduced or eliminated Rb expression or function. Therefore, one aspect of the invention provides a method of generating or regenerating an inner ear sensory cell by contacting the cell with another type of inner ear sensory cell in which the expression level or function of a Rb gene and/or its protein has been eliminated or reduced. In some embodiments the cell in which Rb expression or function has been eliminated or reduced is an intact cell. In another embodiment some portion of the cell is collected (e.g., a cell fraction subsequent to cell lysis) and used in the methods provided. In one embodiment of the invention a method is provided to generate or regenerate inner ear supporting cells by eliminating or reducing the expression level or function of a Rb gene and/or its protein in a hair cell and contacting a supporting cell with the hair cell to generate or regenerate the supporting cell.

In another aspect of the invention a method for restoring hearing or balance to a subject is provided. The subject can be any subject who suffers from or is at risk of suffering from hearing damage, loss of hair cells and/or the symptoms of hearing damage or loss of hair cells. Such a method includes the step of eliminating or reducing the expression level or function of pRb in the inner ear sensory cells of the subject by an amount effective to generate functional, differentiated inner ear hair cells and thereby to restore hearing or balance to the subject. In some embodiments the subject suffers from hearing damage due to a viral infection, noise, a mutation in a gene which causes hair cell death, or ototoxic drug exposure. In some embodiments this method is performed in vivo, while in other embodiments the cells are manipulated in vitro and then provided to the inner ear of the subject. Therefore, in another aspect of the invention a method for restoring hearing or balance to a subject by providing to the subject in need thereof functional, differentiated inner ear hair cells generated by the elimination or reduction of the expression level or function of Rb in inner ear sensory epithelial cells is also provided.

Inner ear sensory cells include any cell that can generate a functional, differentiated hair cell. Inner ear sensory cells include progenitor cells, supporting cells and hair cells. In some embodiments the supporting cells and/or hair cells are cells of the vestibular or auditory system. Cells of the vestibular system include cells of the utricle, saccule maculae and three crista. In other embodiments the supporting and/or hair cells are the cells of the auditory system. Cells of the auditory system include the cells of the cochlea.

The compositions and methods of the invention can also be used to generate or regenerate neuronal cells. In one aspect of the invention, therefore, a method for generating or regenerating neuronal cells is provided wherein the expression level or function of a Rb gene and/or its protein is reduced or eliminated in the neuronal cells. In one embodiment the neuronal cell is an inner ear neuronal cell. In another embodiment the neuronal cell is of the central nervous system. In another embodiment the neuronal cell is of the peripheral nervous system.

The retinoblastoma gene and/or retinoblastoma protein includes any of the genes and/or proteins that are part of the retinoblastoma family. In one embodiment the retinoblastoma gene and/or retinoblastoma protein is Rb1/Rb/p105. In another embodiment the retinoblastoma gene and/or retinoblastoma protein is Rbl1/p107. In yet another embodiment the retinoblastoma gene and/or retinoblastoma protein is RB2/Rbl2/p130. The retinoblastoma gene and/or retinoblastoma protein also includes any of the genes and/or proteins that are involved in the retinoblastoma pathway.

The reduction or elimination of the expression level or function of a retinoblastoma gene and/or its protein can be accomplished with a variety of agents. In some embodiments the agents are Rb-binding molecules. Rb-binding molecules are molecules that bind to the retinoblastoma gene or the retinoblastoma protein. Such molecules include, in some embodiments, antisense oligonucleotides, RNAi or siRNA molecules, intrabodies, adenovirus E1A or SV40 T-antigen. In some embodiments the molecules are Rb-binding polypeptides, such anti-Rb antibodies or anti-Rb antibody fragments. In one embodiment the elimination or reduction of the expression level or function of retinoblastoma gene and/or its protein can be accomplished by eliminating the Rb gene. In another embodiment the Rb gene and/or its transcripts are bound by a Rb-binding molecule, e.g., Rb-binding nucleic acids. In still another embodiment methods and compositions are contemplated whereby nucleic acids that eliminate or reduce the expression level of Rb are produced through the use of hair cell specific promoters. In one embodiment, the production of antisense oligonucleotides, RNAi or siRNA molecules can be placed under the control of the hair cell specific promoter. Hair cell specific promoters include, but are not limited to Brn 3.1, Math-1, myosin VIIa and Lhx3. In one embodiment of the invention an expression vector or plasmid which contains a hair cell specific promoter is provided. In still another embodiment a method of administering the expression vector or plasmid containing a hair cell promoter in order to generate or regenerate inner ear sensory cells is also provided. In yet a further embodiment in vitro methods for using expression vectors or plasmids containing a hair cell promoter for generating or regenerating inner ear sensory cells are also provided.

The reduction or elimination of the expression level or function of retinoblastoma gene and/or its protein can also be accomplished in yet other embodiments with kinase activators, cyclin-dependent kinases (CDKs), and/or agents that inhibit the activity of kinase inhibitors (e.g., histone acetyltransferase (HAT) inhibitors). In another aspect of the invention compositions of the agents provided herein and a pharmaceutically acceptable carrier are provided. The methods and compositions provided herein can, in some embodiments, include at least 2, 3, 4, 5, 6, or more different agents that reduce or eliminate the expression level or function of a Rb gene and/or its protein product.

The methods provided herein can also further include eliminating or reducing the expression level or function of other molecules, such as other cell cycle regulators. In some embodiments the expression level or function of p27kip1, p57kip2, Isl-1, a Notch family protein or a MAPK-JNK family protein is reduced or eliminated in these methods.

In another aspect of the invention a functional, differentiated inner ear hair cell line is provided. In some embodiments the functional, differentiated inner ear hair cell line is composed of functional, differentiated inner ear hair cells with reduced or eliminated expression level or function of Rb. The functional, differentiated inner ear hair cells in other embodiments can further have reduced or eliminated expression level or function of p27kip1, p57kip2, Isl-1, a Notch family protein or a MAPK-JNK family protein.

In still another aspect of the invention an inner ear sensory epithelial cell line, wherein the expression level or function of Rb is eliminated or reduced is also provided. In some embodiments the inner ear sensory epithelial cell line is an inner ear progenitor, supporting or hair cell line. In some embodiments the cell line lacks a thermolabile variant of the large T antigen that is stable at 33° C. The inner ear sensory epithelial cell line in some embodiments also can further have reduced or eliminated expression level or function of p27kip1, p57kip2, Isl-1, a Notch family protein or a MAPK-JNK family protein.

In yet another aspect of the invention an inner ear sensory epithelial cell, wherein the expression level or function of pRb is reduced or eliminated, is also provided. In some embodiments the inner ear sensory epithelial cell is a functional, differentiated inner ear hair cell. In other embodiments the inner ear sensory epithelial cell is a supporting cell. In yet other embodiments the inner ear sensory epithelial cell is a progenitor cell. In still other aspects of the invention compositions of inner ear sensory epithelial cells with reduced or eliminated Rb expression level or function and a pharmaceutically acceptable carrier are provided. Compositions provided herein, in some embodiments, can further comprise a therapy for treating hearing damage or ameliorating the symptoms of hearing damage. In some embodiments the therapy is a hearing aid or cochlear implant.

In still another aspect of the invention a screening method for identifying compounds for generating, regenerating or protecting hair cells by contacting a candidate compound with a sample containing cells of a functional, differentiated inner ear hair cell line, and determining if the candidate compound affects the production of or protects the functional, differentiated inner ear hair cells is provided. In other aspects a screening method for identifying compounds for inducing supporting cells or progenitor cells to become hair cells by contacting a candidate compound with a sample containing cells of an inner ear supporting or progenitor cell line, and determining if the candidate compound induces the supporting cells to become hair cells is also provided. In some embodiments the supporting cells are cells where the expression level or function of Rb is eliminated or reduced. In still other embodiments the cells lack a thermolabile variant of the large T antigen that is stable at 33° C.

Yet another aspect of the invention provides a method for generating or regenerating inner ear sensory epithelial cells (e.g., functional, differentiated inner ear hair cells) by eliminating or reducing the expression level or function of Isl-1 in inner ear sensory epithelial cells by an amount effective to generate inner ear sensory epithelial cells. In another aspect the method is a method for restoring hearing or balance in a subject by eliminating or reducing the expression level or function of Isl-1 in inner ear sensory cells. These methods can be performed in vivo or in vitro. In other aspects of the invention inner ear sensory epithelial cell lines, wherein the expression level or function of Isl-1 is eliminated or reduced, are provided. The cells, compositions thereof and methods of screening with such cells are also provided in other aspects of the invention.

Use of the foregoing cells, cell lines and agents in the preparation of medicaments, particularly for the treatment of hearing loss or loss of balance, is also provided according to the invention.

Each of the limitations of the invention can encompass various embodiments of the invention. It is, therefore, anticipated that each of the limitations of the invention involving any one element or combinations of elements can be included in each aspect of the invention.

These and other aspects of the invention, as well as various advantages and utilities, will be more apparent with reference to the detailed description of the preferred embodiments.

### BRIEF DESCRIPTION OF THE FIGURES

FIG. 1 provides a diagram of the utricle.

FIG. 2 provides the list of genes identified as being primarily expressed in the sensory epithelia.

FIG. 3 provides the list of cell cycle regulators that were identified and clustered into 15 groups by K-mean analysis.

FIG. 4 provides the results from the K-mean cluster analysis of expression profiles of the developing utricles (Panel A). 2331 genes were classified into 15 clusters. Multiple cyclin genes (CDK4, cyclin A2, B1 and B2) showed high levels of expression during early development (cluster 4). Rb members were assigned to three distinct clusters (Panel B) which showed down-regulation of p107 during development; consistent expression level of Rb; and up-regulation of p130. The expression profiles of the three genes closely follow their known roles in the cell cycle (bottom graph). The samples analyzed are indicated in cluster 4 (Panel B). Ht-heart; MEU-cell mouse embryonic utricular cell line; MEU (RA)-MEU treated with retinoic acids; Re-retina; U-utricle; U+Sac-utricle and saccule; U-Se-utricle sensory epithelia. In situ hybridization of selected cell growth regulators (Panels C-E): MAX-interacting protein 1 Mxi1 (Panel C); v-ab1 Abelson murine leukemia oncogene 1 (Panel D) and Jun-B oncogene (Panel E). The prominent hair cell expression of the genes confirmed the conclusions in the GeneChip studies. Ut-utricle; Sac-saccule; Amp-ampulla; HC-hair cell; SC-supporting cell.

Scale bars=50 μm.

FIG. 5 shows the expression pattern of Rb (Panels A-H) and p130 (Panels I-L) in the developing inner ear. Anti-Rb antibody stained widely in E12.5 otocyst (Panel A), and had prominent hair cell labeling from E14.5 up to the adult (Panels B-G). Panel B shows that Rb is absent in the Rb^{-/-} utricle. p130 shows minimal expression at early stages (Panels I-J), and an increased expression in the hair cells at later stages (Panels K-L). Panels M-P provide the confocal images of phalloidin labeling of the hair bundles in the whole utricles (Panels M and N) and mid-turn of the cochlea (Panels O and P). Panel N shows circled areas which mark the regions with significantly less (left) or more (right) hair bundles in the Rb^{-/-} utricle. Panel P shows the drastic increase in the number of the hair cells evident in the Rb^{-/-} cochlea. The Rb^{-/-} has as many as 3-4 rows of inner hair cells and 8-10 rows of outer hair cells. Most of the Rb^{-/-}cochlear hair cells have hair bundles, with irregular orientation. Ot-otocyst; Sac-saccule; Ut-utricle; Coch-cochlea; HC-hair cell; SC-supporting cell; IHC-inner hair cell; OHC-outer hair cell.

Scale bars=50 μm.

FIG. 6 illustrates the results from the in situ hybridization confirming the expression of Col1A1.

FIG. 7 shows dividing hair cells in the Rb^{-/-} mice from the results of PCNA and myosin VIIa double-labeling of the E13.5 utricle (Panels A-F), E18.5 utricle (Panels G-L) and E18.5 cochlea (Panels M-R). PCNA labeled the dividing cells whereas myosin VIIa labeled the hair cells. Panels A-C, Panels G-I and Panels M-O are from the Rb^{+/-} cells; Panels D-F, Panels J-L and Panels P-R are from the Rb^{-/-} cells. In all cases, prominent PCNA staining was observed in virtually all of the hair cells in the Rb^{-/-} mice, and no PCNA labeling was found in any of the control hair cells. There was a mild increase in the number of hair cells in the E13.5 Rb^{-/-} utricle (Panel E) and drastically more hair cells in the E18.5 Rb^{-/-} utricle (Panel K), indicating continuous cell division of the hair cells between E13.5 and E18.5. Multi-rows of the inner and the outer hair cells were found in the E18.5 Rb ^{-/-} cochlea (Panel Q). The brackets (Panel P and Panel R) indicate PCNA labeled supporting cells. Panel S provides the results of the Brn-3.1 and DAPI labeling. Hair cells in the M phase of the cell cycle (arrowheads) in the Rb^{-/-} utricle and DAPI labeled condensed chromosomes being separated to two daughter cells are shown. The hair cells in M phase are also indicated by arrowheads in (Panel L), which shows co-localization of PCNA and myosin VIIa.

Scale bars: 50 μm (Panels A-R); 10 μm (Panel S).

FIG. 8 illustrates the results of the immunostaining of p27kip1 (Panels A and C) and Rb (Panels B and D) in the normal cochlea and the in situ hybridization of p27kip1 in the control (Panels E) and G) and Rb^{-/-} cells (Panels F and H). In the E14.5 otocyst the initial downregulation of p27kip1 in the future hair cell region (Panel A) correlates with mildly increased expression of Rb (Panel B) in the same region. Brackets denote a zone of non-proliferating cells within the future organ of Corti. Note the cells with slight downregulation of p27kip1 and upregulation of Rb (Panel C). Absence of p27kip1 expression coincides with upregulation of Rb (Panel D) in the hair cells in the E16.5 cochlea. p27kip1 (Panel F) was downregulated in some supporting cells of Rb^{-/-} cochlea (arrows), compared to the control (Panel E). Note that the mRNA of p27kip1 is located in the apical region of the cells. Little p27kip1 expression could be detected within the control and Rb ^{-/-} utricles. Ot-otocyst; Coch-cochlea; Ut-utricle; HC-hair cell; SC-supporting cell.

Scale bars: 25 μm.

FIG. 9 provides the results from BrdU labeling of the dividing hair cells in the Rb^{-/-} mice. Extensive BrdU labeling in the hair cells was only in the Rb ^{-/-} organ of Corti (Panels D-F) and utricle (Panels J-L), where no BrdU labeling was found in the hair cells in the control (Panels A-C) and (Panels G-I). Note the hair cells have in general weak BrdU labeling (arrows show examples), in contrast to some strong BrdU labeling in the supporting cells (arrowheads), indicating continuous hair cell division after initial incorporation of BrdU. Also BrdU labeling of the cochlear supporting cells only occurred in the Rb^{-/-} mice. Hair cells were labeled with myosin VIIa.

Scale bars=50 μm.

FIG. 10 provides the results of the immunostaining of p27kip1 (Panels A and B) in the normal cochleas, and in situ hybridization of p27kip1 (Panels C-F) and p57kip2 immunostaining (Panels G-J) in the control and Rb^{-/-} mice. Panel A shows that in the E14.5 otocyst there was an initial down regulation of p27kip1 in the future hair cell region. A bracket denotes a zone of non-proliferating cells within the future organ of Corti. Panel B shows that p27kip1 was completely absent in the cochlear hair cells at E16.5. Panel D shows that p27kip1 was down-regulated in the supporting cells of the Rb^{-/-} cochlea (arrows), compared to the control (Panel C). Note that the mRNA of p27kip1 is located in the peri-nuclear region of the cells. Panels E and F illustrate that the low level of p27kip1 expression could be detected in the control and Rb^{-/-} utricles. Panels G-H illustrate the prominent expression of p57kip2 in the outer hair cells of the control cochlea. Panels I-J show that the expression of p57kip2 was severely reduced in some, while was completely absent in rest of the Rb^{-/-} outer hair cells. Ot-otocyst; Coch-cochlea; Ut-utricle; HC-hair cell; SC-supporting cell. Sac-saccule.

Scale bars: 25 μm.

FIG. 11 shows the results from the hair cells labeled with different hair cell markers. Panels A and B provide the results from the espin staining showing the labeled hair bundles in both the control (Panel A) and Rb^{-/-} utricles (Panel B), indicated by the arrows. Panels C and D show tubulin labeled nerve fibers found to surround the hair cells in the control (Panels C) and the Rb^{-/-} utricles (Panels D) (arrows). Note the labeling surrounding the hair cells beneath the apical hair cells in the Rb^{-/-} utricle. There is a clear disorganization of the nerve fibers in the Rb^{-/-} utricle. Lhx3 and myo7a label the hair cells, in the nuclei and cytoplasm, respectively. Panels E and G show that one layer of the Rb^{+/-} utricle hair cells was above the supporting cell layer; Panels F and H show 3-4 layers of hair cells in the Rb^{-/-} utricle. Among those in the supporting cell layer, some have typical cylindrical shape of supporting cell nuclei (arrowheads). Arrows indicate the hair cells that were facing away from apical lumen.

Scale bars=50 μm.

FIG. 12 illustrates the results of the functional analysis of pRb^{-/-} hair cells. Panels A-F show the DIC and FM1-43 uptake images of the E18.5 pRb^{+/-} and pRb^{-/-} utricular hair cells. Most of the pRb^{-/-} hair cells, like the pRb^{+/-} cells, showed FM1-43 uptake (examples of co-localization of hair bundles and Fma-43 labeling are shown by the arrows, indicating that they have functional mechanotransduction channels). Panels G-H show that the transduction apparatus was functional in the pRb^{-/-} mice at E18.5. Panel G shows that transduction currents were elicited in wild type (top) and pRb^{-/-} (middle) littermates by step deflections of the hair bundle (bottom). Adaptation of the transduction current was visible in response to positive hair bundle deflections as decay of the transduction current. In responses to negative hair bundle deflections adaptation visible in the large response at the end of the step. The wild type response was typical of transduction currents in neonatal mice (Vollrath and Eatock, 2003). However, all transduction currents in the pRb^{-/-} mice were small: mean maximal transducer current±SEM=14.2 pA±2.9 (n=4). Panel H shows the normalized I(X) relations for the wild type and pRb^{-/-} mouse transduction currents shown in Panel G. The two hair cells shown in this example had similar operating ranges.

Scale bars: 50 μm.

FIG. 13 illustrates the results of the Isl-1 and myosin VIIa labeling of the E13.5 (Panels A-F) and E18.5 (Panels G-L) utricles. At E13.5, Isl-1 equally labeled the supporting cells and the hair cells in the control utricle (Panels A-C), whereas Isl-1 primarily labeled the supporting cells in the Rb^{-/-} utricle with markedly reduced labeling in the hair cells (Panels D-F). Notice that similar to the control, many supporting cells were present in the E13.5 Rb^{-/-} utricle. At E18.5, Isl-1 labeled the supporting cells both in the control (Panel G) and the Rb^{-/-} (Panel J) utricle striola regions. In the same region Isl-1 labeling was absent in the Rb^{-/-}hair cells (Panel L), while it remained at a lower level in the control hair cells (Panel I). Arrows indicate the hair cells with an orientation facing away from the lumen. Arrowheads show the cylindrical shape of the hair cell nuclei in the supporting cell zone, implying that the cells may have been derived from the supporting cells.

Scale bars: 50 μm.

FIG. 14 illustrates the results of the Lhx3 and Brn-3.1 labeling of the Rb^{-/-} utricle (Panels A-C). Three cells in the supporting cell region were Brn-3.1 positive and Lhx 3 negative (circles), indicating that they were newly differentiated hair cells. Panels D-F illustrate the Isl-1 and myosin VIIa staining of the Rb utricle. The supporting cells, with downregulated Isl-1 expression, were also myosin VIIa negative (circle), suggesting the cells may have been in the process of becoming hair cells. The arrow points to a cell within the same region expressing weak myosin VIIa, indicating it was a newly differentiated hair cell.

Scale bars: 50 μm.

FIG. 15 shows the results of p27, myosin VIIa and DAPI staining of the E18.5 control (Panels A-D) and pRb (Panels E-H) cochleas. The arrowhead points to a cell in the Rb^{-/-} cochlea, in the region of the supporting cells, with weak myosin VIIa expression and downregulated p27 expression. It is likely that this cell was a newly differentiated hair cell, from one of the supporting cells. No such event was observed in the control.

FIG. 16 provides evidence of supporting cell to hair cell induction. Panels A-C show that all the Rb^{+/-} utricle hair cells were above the supporting cell layer; Panels D-F show that the nuclei of R^{-/-} utricle hair cells, in the supporting cell layer, have the typical cylindrical shape of supporting cell nuclei (arrowheads). Arrows indicate that the hair cells were facing away from the apical lumen. Panels G-I show a hair cell in the supporting cell layer in an Rb^{-/-} cochlea. Myosin VIIa labeling was weak in the hair cell, indicating a newly derived hair cell which coincided with the absence of p27kip1 expression. Panels J-K provide the results of the in situ hybridization of Notch1 in the Rb^{-/-} and Rb^{+/-} utricle. The mRNA of Notch1 was concentrated in the apical surface of the epithelium between the hair cells. The expression of Notch1 was greatly reduced in the Rb^{-/-} utricle.

FIG. 17 shows the labeling of pRb in the developing mouse inner ear. Panel A illustrates that an anti-pRb antibody showed immunoreactivity ubiquitously in the E12.5 otocyst. pRb labeling was prominent in hair cells of E14.5 saccule (Panel B), and of P6 utricle (Panel C). Myosin VIIa (Myo7a) stained hair cells. Distinct pRb staining was in cochlear hair cells at E16.5 (Panel D) and in adult (Panel E). pRb immunoreactivity was detected prominently in both hair cells and supporting cells of adult utricle (Panel F). Ot-otocyst; Sac-saccule; Ut-utricle; Coch-cochlea; HC-hair cells, SC-supporting cells, IHC-inner hair cells and OHC-outer hair cells.

Scale bars=25 μm.

FIG. 18 shows the expression of collagen 1A1 (Col1A1) in the inner ear by in situ hybridization. Panel A shows that Col1A1 was detected ubiquitously throughout otocyst at E11.5, indicating that Rb1 could be deleted in otocyst as early as E11.5. Panel B demonstrates that Col1A1 expression was detected, but at a reduced level, in both hair cells and supporting cells of E13.5 utricle. Ot-otocyst; Ut-utricle; HC-hair cell; SC-supporting cell.

Scale bars=25 μm.

FIG. 19 shows the expression of Rb1 in the inner ear and increased hair cell numbers in the Col1A1-pRb^{-/-} mice. Panels A and B show that an anti-pRb antibody primarily stained hair cells in E18.5 control utricle; an antibody to myosin-7a (Myo7a) marked hair cells. Panels C and D show that pRb was absent in the E18.5 Col1A1-pRb^{-/-} utricle. Note multiple-layer hair cells in Col1A1-pRb^{-/-} utricle. Basal lamina marked with dashed lines. Confocal images of rhodamine phalloidin-labeled hair bundles in the E18.5 utricular macula (Panels E and F) and mid-turn of the cochlea (Panels G and H). The distribution of hair cells in Col1A1-pRb^{-/-} utricle was abnormal, as indicated by clustered hair bundles [round circles in (Panel F)], in contrast to the normal mosaic pattern in control (Panel E). Panels G and H show that inner hair cells (arrows) and outer hair cells (arrowheads) in cochlea remained separated by pillar cells, which do not have hair bundles. Uniform orientation of the hair bundles was altered in Col1A1-pRb^{-/-} cochlear hair cells (Panel H). Ut-utricle; Coch-cochlea; IHC-inner hair cell; OHC-outer hair cell.

Scale bars=25 μm.

FIG. 20 shows sensory progenitor cells and hair cells undergoing mitosis in Col1A1-pRb^{-/-} mice. An anti-myo7a antibody labels hair cells; confocal images. Panels A, E and I show that in E18.5 control utricular macula, BrdU labeling was not found in hair cells, but appeared in some supporting cells. Panels B, F and J show that in Col1A1-pRb−/− utricular macula, BrdU labeling appeared in both hair cells and supporting cells. Panels C, G and K show no BrdU labeling in control cochlear hair cells or supporting cells. Panels D, H and L show BrdU labeling of Col1A1-pRb^{-/-} cochlear hair cells and supporting cells. Overall hair cell labeling was weaker (arrows) than supporting cells (arrowheads) (Panels F, H, J and L). Panel M shows that no BrdU labeling was in control progenitor cells in the ZNPC (demarcated by dashed lines) of the primordial organ of Corti at E13.5, whereas BrdU staining was in Col1A1-pRb^{-/-} progenitor cells (Panel N). Panel O shows hair cells in M-phase of cell cycle, as shown by cytoplasmic like labeling for Brn-3.1 and condensed nuclear labeling by DAPI (arrows). Inset shows a M-phase hair cell with Brn-3.1 alone and Brn-3.1 plus DAPI labeling. Ut-utricle; Coch-cochlea.

Scale bars=25 μm (Panels A-N) and 10 μm (Panel O).

FIG. 21 shows the cell-specific proliferation of E18.5 cochlear supporting cells. Panel A shows that an anti-S100A1 antibody labeled Deiters' cells (DC), as well as inner hair cell and phalangeal cells (PHC) in control cochlea. The Pillar cells (PC) were unlabeled. Panel B shows that the same labeling was detected in the Col1A1-pRb^{-/-} cochlear supporting cells. However, there was an increase in the number of Deiters' cells in Col1A1-pRb^{-/-} cochlea (an average of 7-9 vs. 4-5 in control). In Col1A1-pRb^{-/-} cochlea, only two Pillar cells were present, indicating there was no proliferation. Panel C shows that an anti-p75ntr antibody showed labeling in the apical region of Pillar cells in control (red between IHC and OHC), and similar labeling was in the Col1A1-pRb^{-/-} Pillar cells (Panel D). p27kip1 labeled all the cochlear supporting cells. OHC-outer hair cells and IHC-inner hair cells.

Scale bars=25 μm.

FIG. 22 shows the results of PCNA labeling of proliferating hair cells in the E13.5 and E18.5 Col1A1-pRb^{-/-} inner ear. Panels A-C show that in the E13.5 Col1A1-pRb^{+/-} utricle, no PCNA labeling was in hair cells. PCNA however labeled supporting cells. Panels D-F show that strong PCNA labeling was in virtually all the hair cells in E13.5 Col1A1-pRb^{-/-} utricle. Panels G-I show that in the E18.5 Col1A1-pRb^{+/-} utricle, no PCNA labeling was in hair cells. Scattered PCNA labeling was seen in some supporting cells. Panels J-L show that strong PCNA labeling was in most hair cells in E18.5 Col1A1-pRb^{-/-} utricle. Panels M-O show that no PCNA labeling was in either Col1A1-pRb^{+/-} cochlear hair cells or supporting cells. Panels P-R show that there was strong PCNA labeling in almost all the Col1A1-pRb^{-/-} cochlear hair cells and in some supporting cells. Myosin VIIa (myo7a) stained the hair cells. HC-hair cells; SC-supporting cells and ST-stroma tissue.

Scale bars=25 μm.

FIG. 23 shows hair cells labeled with differentiating hair cell markers. Panels A-D show Lhx3 labels hair cell nuclei. Antibodies to espin labeled hair bundles (arrows) in control (Panel A) and Col1A1-pRb^{-/-} utricles (Panel B). Panels C and D show that antibodies to Ptprq labeled hair bundles (arrows) in control (Panel C) and Col1A1-pRb^{-/-} cochleas (Panel D). Panels E and F show that antibodies to tubulin labeled nerve fibers surrounding hair cells marked with myo7a (arrows) in control (Panel E) and the Col1A1-pRb^{-/-} cochleas (Panel F). Note labeling surrounding multiple inner hair cells in the Col1A1-pRb^{-/-} cochlea (Panel F).

Scale bars=25 μm.

FIG. 24 shows the synaptophysin labeling of surrounding hair cells. Myo7a labeled hair cells. Antibodies to synaptophysin labeled nerve terminals surrounding hair cells (arrows) in E18.5 control (arrows in Panels A and C) and the Col1A1-pRb^{-/-} cochleas (arrows in Panels D and F).

Scale bars=25 μm.

FIG. 25 shows the functional mechanotransduction by Col1A1-pRb^{-/-} and control hair cells at E18.5. Panels A-F show FM1-43 accumulation by utricular hair cells. After a 1 min exposure to FM1-43, most hair bundles (DIC images, Panels A and D) were labeled with FM1-43 (Panels B and E) in both control (Panels A-C) and Col1A1-pRb^{-/-} (Panels D-F) mice, indicating that these cells had functional mechanotransduction channels. Arrows indicate clear-labeled bundles. Panel G shows transduction currents elicited in control (top) and Col1A1-pRb^{-/-} (middle) littermates by step deflections of the hair bundle (bottom). Adaptations of the transduction currents in response to positive and negative hair bundle deflections were revealed. The wild type response is typical of transduction currents in neonatal mice (M. A. Vollrath, R. A. Eatock, J Neurophysiol 90, 2676 (2003)). However, transduction currents in Col1A1-pRb^{-/-} mice were small: peak transducer current (mean±SEM) was 14.2±2.9 pA (n=4). Panel H shows the normalized I(X) relations for the control and Col1A1-pRb^{-/-} transduction currents shown in Panel G. These two hair cells had similar operating ranges.

Scale bars=10 μm.

FIG. 26 shows the results of anti-activated caspase 3 labeling in the organ of Corti. Panels A and B demonstrate that caspase 3 labeling shows no positive cells in the Col1A1-pRb^{+/-} organ of Corti, with an exception of positive labeling in mesenchyme cell (arrow). Panels C and D show that no caspase 3 immunoreactivity was observed in the Col1A1-pRb^{-/-} organ of Corti. C3-caspase 3; Is1-Islet-1.

Scale bars=25 μm.

FIG. 27 shows cell cycle re-entry by postmitotic hair cells, after acute deletion of Rb1 gene. Panels A and B show the E17.5 and Panels C and D show P10 floxP-pRb utricles infected with adenovirus carrying GFP as controls, and then cultured with addition of BrdU. All hair cells are pRb positive and BrdU negative. The two BrdU positive cells (Panels A and B) are not hair cells. Panels E and F show E17.5 and Panels G and H show P10 floxP-pRb utricles infected with adenovirus carrying Cre/GFP. Cell cycle re-entry by the infected hair cells (pRb) negative was shown by BrdU labeling (Panels F and H). As an internal control, no BrdU labeling was in the uninfected hair cells (pRb positive, arrows).

Scale bars=25 μm.

FIG. 28 demonstrates the cell cycle re-entry of postmitotic hair cells, after acute deletion of Rb1 gene. E17.5 (Panels A and B) and P10 (Panels C and D) floxP-pRb utricles were infected with adenovirus carrying GFP as controls and then cultured, with the addition of BrdU. All hair cells are pRb positive and BrdU negative. The two BrdU positive cells (Panels A and B) are not hair cells. E17.5 (Panels E and F) and P10 (Panels G and H) floxP-pRb utricles were infected with adenovirus carrying Cre/GFP. Cell cycle re-entry by the infected hair cells (pRb negative) was shown by BrdU labeling (Panels F and H). As an internal control, no BrdU labeling was in the uninfected hair cells (pRb positive, arrows).

Scale bars=25 μm.

FIG. 29 shows the proliferation of hair cells in postnatal Brn-Cre-pRb utricle. PCNA labeled virtually all hair cells at P4. PCNA labeling was decreased from P17 to 6-week Brn-Cre-pRb, and no PCNA labeling was observed in 3-month-old Brn-Cre-pRb hair cells.

Scale bars=25 μm.

FIG. 30 shows that hair cells are functional in postnatal Brn-Cre-pRb mice. Transduction current recordings showed robust currents in P4 Brn-Cre-pRb hair cells (left, bottom), which was similar to that in the control (left, top). In 3-month-old mice FM1-43 uptake experiments showed labeling in both Brn-Cre-pRb (right, top) and control (right, bottom) hair cells.

FIG. 31 shows the induction of supporting cell proliferation in Brn-Cre-pRb cochlea. PCNA labeled both hair cells and supporting cells in Brn-Cre-pRb and control utricle (left and right, top panel). PCNA did not label control cochlea (left, bottom panel) whereas it labeled both hair cells and supporting cells of Brn-Cre-pRb cochlea (right, bottom panel).

Scale bars=25 μm.

FIG. 32 shows sensory progenitor cells undergoing mitosis in Col-pRb ^{-/-} mice (Panel A). No BrdU labeling was seen in the control progenitor cells in the ZNPC (demarcated by dashed lines) of the primordial organ of Corti at E13.5, whereas BrdU staining was in Col1A1-pRb^{-/-} progenitor cells (Panel B).

Scale bars=25 μm.

### DETAILED DESCRIPTION OF THE INVENTION

The retinoblastoma protein (pRb) is well studied for its multiple roles in tumorigenesis, terminal exit of cell cycle, protection from apoptosis and differentiation (Classon and Harlow, 2002; Lipinski and Jacks, 1999). More than 100 proteins have been shown to interact with pRb (Morris and Dyson, 2001). Underphosphorylated pRb interacts with its cellular targets, most notably, the E2F family of transcription factors (E2F1-E2F3) and suppresses their transcription activities.

Phosphorylation of pRb by cyclin-dependent kinases (CDKs) releases pRb from its binding to E2F members, enabling them to regulate cell proliferation by promoting transition from the G1 to S phase of the cell cycle (Dyson, 1998). Activity of CDK depends upon binding with cyclin partners and is inhibited by cyclin-dependent kinase inhibitors, such as p27kip1 (Dyson, 1998). Homozygous Rb1 knockout animals are embryonic lethal between E13-E15, with severe defects in lens development, hematopoiesis, myogenesis, osteogenesis and neurogenesis (Classon and Harlow, 2002; Ferguson and Slack, 2001; Thomas et al., 2001). It has been shown that pRb interacts with bHLH protein Id2, an important regulator for proliferation and differentiation (Lasorella, 2002; Lasorella, 2000). Furthermore the genomic sequence of the Jagged2 (a ligand for Notch1) promoter contains a potential binding site for E2F, suggesting pRb may also influence the Notch pathway (Deng, 2000).

Rb plays a key role in neurogenesis. In the nervous system of Rb null mice the neuronal differentiation of proliferating precursors was impaired as manifested by decreased expression of neuronal markers such as neurotrophin receptors TrkA, TrkB and βII tubulin. Ectopic mitoses were found in many regions of the brain and a large number of cells underwent apoptosis in both the central and peripheral nervous systems (Ferguson and Slack, 2001; Yoshikawa, 2000). In pRb null mice cells underwent ectopic mitoses and subsequent apoptosis in both the central and peripheral nervous systems (Jacks et al., 1992; Macleod et al., 1996). Transgenic studies, using neuron specific Ta1 α-tubulin promoter driving a lacZ reporter gene in Rb null mice, showed widespread abnormal neuronal development including the retina, the neocortex and the olfactory epithelium. It has also been shown that Rb becomes essential immediately after neuron fate determination and that lack of Rb causes virtually all neuron populations to undergo apoptosis (Gloster et al., 1999; Slack et al., 1998). However, recent studies using conditional mice that specifically deleted pRb in the CNS, showed an increase in neuronal populations due to aberrant S phase entry (Ferguson et al., 2002; MacPherson et al., 2003; Marino et al., 2003). Interestingly, there was normal differentiation for some neurons without apoptosis. Rb therefore, seems to have multi-functional roles in the CNS: it is required for the cell cycle arrest in many cell types in the CNS, and it is also necessary for the differentiation of sub-cell types. However, none of the Rb family members has been previously studied in the inner ear.

Molecules with the potential to control cell cycle arrest in inner ear sensory cells were identified with a functional genomics approach in combination with studies of developing utricles. This approach had distinct advantages. A functional genomics approach provided a global view of the expression profiles of the cell cycle regulators during inner ear development, enabling the identification of interesting candidates for in-depth characterization. Also, the selection of the utricle as the sample of choice, together with using the sensory utricular epithelium, greatly reduced the complexity of analysis and allowed the association of the expressed genes with the sensory epithelial cells. With this approach a list of genes with the potential to play a role in the maintenance of cell cycle arrest of inner ear sensory cells was defined.

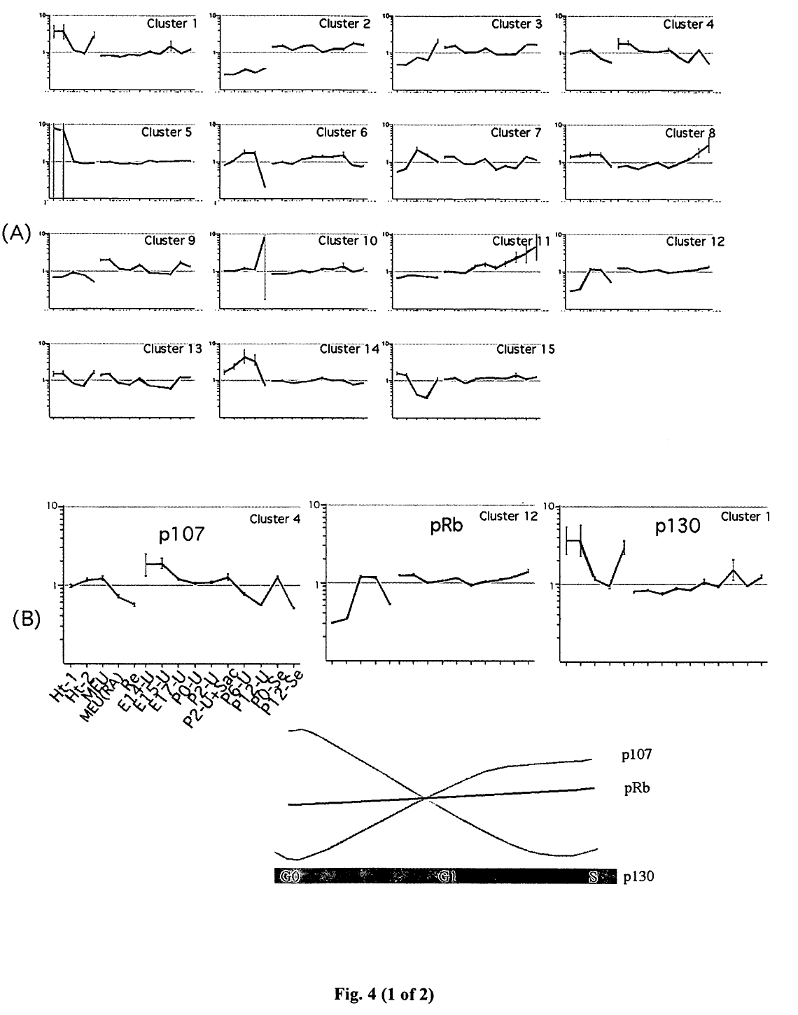

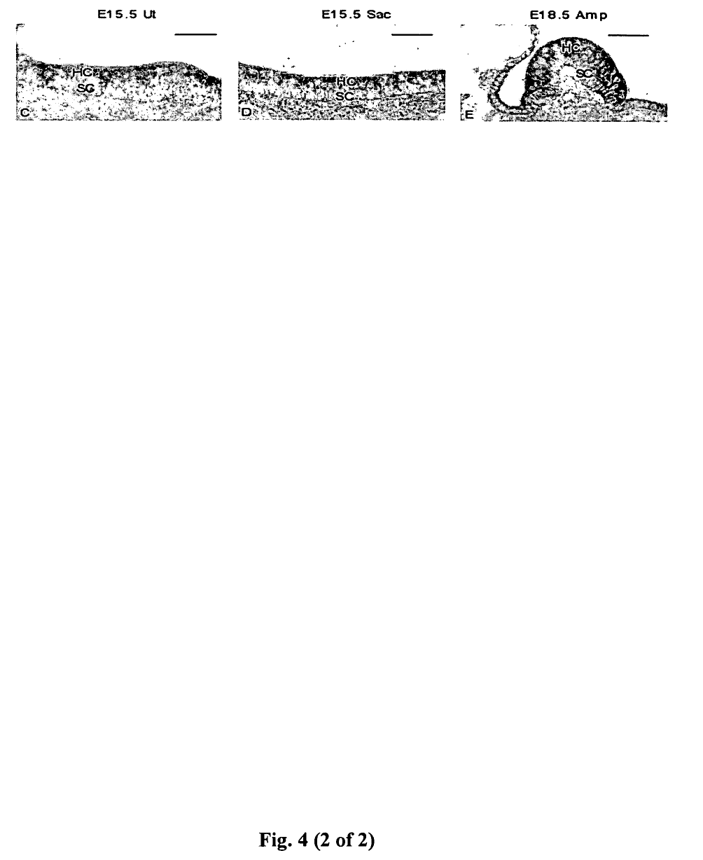

The microarray expression profile analysis identified over 80 cell cycle regulators, including many negative cell growth controllers with expression in the utricle. Their presence in the developing utricular sensory epithelium and their distinct expression patterns from cluster analysis suggest their specific roles in the context of utricle development. For example, p19ink4d, Mxi1, Jun-B and c-fos are all associated with negative regulation of cell growth (Balsalobre and Jolicoeur, 1995; Schlingensiepen et al., 1993; Schreiber-Agus et al., 1998) and share an upregulated expression profile in the developing utricle (FIG. 4 cluster 11), indicating that they may be required in later development, probably in the functions related to the maintenance of cell cycle arrest, similar to the function of p19ink4d. Given the fact that the microarray (GeneChip murine 6500) set used contained probably less than 25% of all the genes encoded in the mouse genome, future studies with higher density chip sets, together with high resolution sampling during the development, should produce additional important genes and associated pathways involved in development.

The results of the studies presented herein indicate that there was a consistent expression pattern of pRb in the inner ear sensory hair cells, from embryo to adult, which strongly suggests that pRb is required throughout hair cell development. The examination of the conditional pRb knockout mouse with pRb expression abolished in the inner ear revealed the primary role of pRb in maintaining the postmitotic state of the hair cells. Also it was demonstrated that one copy of Rb was sufficient to restore full function, as all the analyses done with pRb^{-/+} mice did not reveal any obvious abnormality. This study demonstrated that not only is retinoblastoma protein a key regulator in cell cycle arrest of sensory epithelial cells in the mammalian inner ear, but surprisingly additional differentiated mammalian hair cells can be generated through hair cell division when Rb expression is abolished. The sensory epithelial cells regenerated are differentiated and functional. This is in agreement with recent studies showing that acute loss of pRb in quiescent or senescent mouse embryonic fibroblast cells (MEFs) resulted in cell cycle re-entry (Sage, 2003).

Therefore, by manipulating Rb, to reduce or eliminate its expression level or function, at the gene or protein level, inner ear sensory epithelial cells (both the hair cell and the supporting cell) can re-enter or remain in the cell cycle in vivo or in vitro, leading to the regeneration of sensory epithelial cells in the inner ear, in particular the progenitor, supporting and/or hair cells. Manipulation of Rb, as such, is applicable to developing and fully differentiated sensory epithelial cells. The regenerated functional hair cells have the potential to be used in therapy to restore hearing and balance associated with hair cell damage. Rb is the only gene identified that in its absence enable the highly differentiated and functional hair cells to remain in the cell cycle and produce more hair cells with the same characteristics. Lack of the Rb gene also leads to cycling supporting cells.

Therefore, methods of generating or regenerating sensory inner ear cells, progenitor, supporting, or hair cells that are functional and differentiated, are provided. These methods can be for use in vivo and/or in vitro. The sensory inner ear cells are generated, in such methods, through the elimination or reduction of the expression level or function of a retinoblastoma gene and/or retinoblastoma protein. A “retinoblastoma gene or retinoblastoma protein” refers to any of the members of the retinoblastoma family. The retinoblastoma family members include Rb1/Rb/p105, Rbl1/p107 and RB2/Rbl2/p130. As used herein, the use of “Rb” and like terms is intended to encompass all of the members of the retinoblastoma family, the gene and/or its protein, while the use of “Rb gene” and “pRb” refer specifically to the gene and its protein, respectively. The methods of the invention provided herein can include the elimination or reduction of the expression level or function of each of these retinoblastoma family members either singularly or in any combination. In some embodiments the methods are directed to the reduction or elimination of Rb1/Rb/p105 expression level or function. The methods of the invention provided herein can be performed in any of the inner ear sensory epithelial cells. Such cells include progenitor cells, supporting cells and hair cells. “Progenitor cells”, as used, herein are cells that are capable of self-renewal and can produce progeny cells that are more differentiated than itself. Progenitor cells, therefore, include stem cells. “Supporting cells”, as used herein, include cells that are in direct contact with and/or separate the hair cells. Supporting and hair cells include those of the auditory system (e.g., the ganglion cells of the cochlea) and of the vestibular organs of the inner ear (e.g., the utricle, saccule maculae and three crista).

As used herein “generating” or “regenerating” refers to producing the new desired cells and/or causing cells to re-enter the cell cycle.

The reduction or elimination of the expression level or function of a retinoblastoma gene and/or its protein can be accomplished using a variety of agents (called herein “Rb inhibiting agents”. It will be apparent to one of ordinary skill in the art that agents that reduce or eliminate the expression level or function of Rb include Rb-binding molecules. “Rb-binding molecules” are molecules that bind Rb, such as antisense oligonucleotides (e.g., the antisense oligonucleotides of Bredesen et al., U.S. Pat. No. 5,324,654), RNAi molecules, Rb-binding polypeptides, e.g., anti-Rb antibodies or anti-Rb antibody fragments, intrabodies, small molecules or any other compound that binds to Rb and inhibits its function of maintaining cell cycle arrest. In some embodiments the Rb-binding molecule is adenovirus E1A or SV40 T-antigen that form a complex with pRb between amino acids 393 and 572 and 646 and 772 (Hu et al., 1990). Rb-binding molecules may be isolated from natural sources or synthesized or produced by recombinant means. Methods for preparing or identifying molecules which bind to a particular target, in this instance to Rb, are well-known in the art and are described below. Rb-binding polypeptides, such as antibodies, may easily be prepared by generating antibodies to pRb (or obtained from commercial sources) or by screening libraries to identify peptides or other compounds which bind to pRb.

As provided herein, agents include any molecule that eliminates or reduces the expression level or function of Rb, preferably to generate functional, differentiated hair cells. Agents also include compounds which result in the phosphorylation of Rb, such compounds are kinases, kinase activators or agents that inhibit kinase inhibitor activity. Since unphosphorylated pRb is essential for its function, the proteins such as cyclin-dependent kinases (CDKs) that can phosphorylate pRb may also be used to inactivate pRb. In addition molecules with histone acetyltransferase (HAT) activities such as p300/CBP hinders the phosphorylation of pRb by cyclin-dependent kinases (Chan et al., 2001). Therefore any approach that interferes with HAT activity may reduce pRb activities.

In another aspect of the invention, methods for generating, regenerating or providing regenerated hair cells to a subject in need thereof are provided. Such “a subject in need thereof” is one that has or is at risk of having hair cell damage and/or the symptoms associated with hair cell damage, e.g., hearing loss or loss of balance. As described above, damaged hair cells and supporting cells can be the result of viral infection, overdose of ototoxic drugs, noise, aging, hereditary causes, etc. Therefore, a subject at risk is one who has had exposure to these and/or other risk factors associated with hair cell damage, hearing loss and/or loss of balance. A subject at risk can also be one of advanced age. In one embodiment the subject is 60, 70 80, 85, 90 or more years old. Therefore, a method is provided for restoring hearing or balance to a subject. In a preferred aspect, the method includes eliminating or reducing the expression level or function of Rb in sensory inner ear cells in vivo to cause them to re-enter the cell cycle and to produce new progenitor, supporting or, functional, differentiated hair cells. In another preferable aspect of the invention, the method includes eliminating or reducing the expression level or function of Rb in sensory inner ear cells in vitro and providing these cells to the subject. They can be used to replace the damaged hair cells and/or progenitor or supporting cells in the inner ear, to restore hearing and balance. As Rb is expressed in inner ear sensory epithelial cells from embryo to adult, the generation or regeneration of the sensory epithelial cells can be performed in both early developmental stages or in the adult. The manipulation of the expression level of Rb or the function of pRb can be of short or long duration. The methods provided herein include the use of any agent in an amount effective to reduce or eliminate the expression level or function of Rb. The methods can be performed in any cell of the sensory epithelia that can lead to the production of inner ear hair cells. The result is the generation or regeneration of sensory inner ear cells. The generated/regenerated cells can be the progenitor cells, hair cells, the supporting cells or some combination thereof.

The methods and compositions provided herein can be used alone or in combination with other medical treatments that are used to ameliorate the symptoms associated with hair cell damage and/or hair cell loss. Such treatments include the use of hearing aids and cochlear implants.

Subjects as used herein includes humans, non-human primates, dogs, cats, horses, sheep, goats, cows, rabbits, pigs and rodents.

The methods provided herein can further include reducing or eliminating the expression level or function of proteins that are involved in the Rb pathway. These proteins can be one or more of the proteins of the Rb family. These proteins also can be upstream or downstream of Rb. These proteins, therefore, can include p107(Rbl1), p130(Rbl2), p27kip1, p57kip2, Isl-1, a Notch family protein or a MAPK-JNK family protein.

In another aspect of the invention methods and compositions for the generation or regeneration of neuronal cells through the elimination or reduction of Rb expression or function are provided. Neuronal cells include the neuronal cells of the inner ear, central nervous system or peripheral nervous system. Neuronal cells are predominantly categorized based on their local/regional synaptic connections (e.g., local circuit intemeurons vs. longrange projection neurons) and receptor sets, and associated second messenger systems. There are many different neuronal cell types. Examples include, but are not limited to, sensory and sympathetic neurons, cholinergic neurons, dorsal root ganglion neurons, proprioceptive neurons (in the trigeminal mesencephalic nucleus), ciliary ganglion neurons (in the parasympathetic nervous system), etc. A person of ordinary skill in the art will be able to easily identify neuronal cells and distinguish them from non-neuronal cells such as glial cells, typically utilizing cell-morphological characteristics, expression of cell-specific markers, secretion of certain molecules, etc.

In another aspect of the invention it is the expression level or function of Isl-1 alone or in combination with another gene/protein, that is eliminated or reduced in the supporting or inner ear hair cells, preferably to result in the production of functional, differentiated hair cells. This aspect of the invention is intended to encompass in vitro and in vivo applications.

As one example, antisense oligonucleotides can be used to reduce or eliminate the expression level of Rb. As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA, respectively. The antisense molecules are designed so as to interfere with transcription or translation of a target gene upon hybridization with the target gene or transcript. Antisense oligonucleotides that selectively bind to either a nucleic acid molecule encoding Rb are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding the retinoblastoma family or pathway members (e.g., Rb1, NCBI Accession Nos. M26460, NM_{—}000321; Rbl1/p107, NCBI Accession Nos. L14812, BC069179; RB2/Rbl2/p130, NCBI Accession Nos. BC034490, BC020528) or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., Nat. Med. 1(11): 1116-1118, 1995. Most preferably, the antisense oligonucleotides comprise a complementary sequence of 7, 8, 9, 10, 11, 12, 13, 14, 15, 16, 17, 18, 19, 20, 21, 22, 23, 24, 25, 26, 27, 28, 29 or 30 bases. Although oligonucleotides may be chosen which are antisense to any region of a gene or its mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors. In some embodiments the vectors contain a hair cell specific promoter.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding retinoblastoma protein, together with pharmaceutically acceptable carriers. Antisense oligonucleotides may be administered as part of a pharmaceutical composition. In this latter embodiment, it is preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. In one embodiment it is the vectors that produce antisense oligonucleotides that are administered for the in vivo production of the antisense oligonucleotides.

A reduction or elimination of the expression level of Rb in another method may be achieved by using the technique of RNA interference (RNAi). The use of RNAi involves the use of double-stranded RNA (dsRNA) to block gene expression. (See: Sui, G, et al, 2002, Proc Natl. Acad. Sci U.S.A. 99:5515-5520). The application of RNAi strategies for reducing gene expression specifically is understood by one of ordinary skill in the art.

In one aspect of the invention, a method is provided in which siRNA molecules are used to eliminate or reduce the expression level of Rb. In one embodiment, a cell is contacted with a small interfering RNA (siRNA) molecule to produce RNA interference (RNAi) that reduces expression of one or more Rb molecules. The siRNA molecule is directed against nucleic acids coding for Rb (e.g., RNA transcripts including untranslated and/or translated regions). In a preferred aspect of the invention Rb is Rb1. The expression level of the targeted Rb molecule(s) can be determined using well known methods such as Western blotting for determining the level of protein expression and Northern blotting or RT-PCR for determining the level of mRNA transcript of the target gene.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of a target gene. In one embodiment the siRNA molecule corresponds to a region selected from a cDNA target gene beginning between 50 to 100 nucleotides downstream of the start codon. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of Rb is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. In one embodiment the siRNA is synthesized with plasmids that are controlled by a tissue-specific promoter. Preferably the tissue specific promoter is specific for the inner ear sensory epithelia. In one embodiment the tissue specific promoters are Brn-3.1, Math-1, myosin VIIa and Lhx3.

In one aspect of the invention a vector comprising any of the nucleotide coding Rb is provided, preferably one that includes promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2/V5-GW/lacZ, pENTR/U6, pLenti6-GW/U6-laminshrna and pLenti6/BLOCK-iT-DEST (Invitrogen). These vectors and others are available from commercial suppliers.

The term “high stringency conditions” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C. There are other conditions, reagents, and so forth which can be used, which result in the same degree of stringency. A skilled artisan will be familiar with such conditions, and thus they are not given here.

The methods provided herein, in some embodiments, also encompass the use of other inhibitors of the function of Rb, such as, “dominant negative” molecules. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, given the teachings contained herein of retinoblastoma proteins, one of ordinary skill in the art can modify the sequence of the retinoblastoma protein by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution and/or for retention of an activity. Other similar methods for creating and testing dominant negative variants of a protein will be apparent to one of ordinary skill in the art.

Inhibitors of the function of Rb also include Rb-binding polypeptides. As used herein “Rb-binding polypeptides” are polypeptides which bind to the retinoblastoma protein and inhibit its maintenance of cell cycle arrest, allowing sensory inner ear cells to re-enter the cell cycle, and preferably generate supporting cells and hair cells. Most preferably the hair cells that are generated are functional and differentiated and undergo little or no apoptotic activity. Preferred Rb-binding polypeptides are antibodies, such as monoclonal antibodies, including chimeric, human, or humanized antibodies; single chain antibodies or antigen-binding fragments, such as F(ab′)₂, Fab, Fd, or Fv fragment; and intrabodies.

Antibodies and methods of their production are well known to those of ordinary skill in the art. As used herein, the term “antibody” means not only intact antibody molecules but also fragments of antibody molecules retaining specific binding ability. Such fragments are also well known in the art and are regularly employed both in vitro and in vivo. In particular, as used herein, the term “antibody” means not only intact immunoglobulin molecules but also the well-known active fragments F(ab′)₂, and Fab.

According to one embodiment, the molecule is an intact soluble monoclonal antibody in an isolated form or in a pharmaceutical preparation. An intact soluble monoclonal antibody, as is well known in the art, is an assembly of polypeptide chains linked by disulfide bridges. Two principle polypeptide chains, referred to as the light chain and heavy chain, make up all major structural classes (isotypes) of antibody. Both heavy chains and light chains are further divided into subregions referred to as variable regions and constant regions. As used herein the term “monoclonal antibody” refers to a homogenous population of immunoglobulins which specifically bind to an epitope (i.e. antigenic determinant) , e.g., of pRb.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)₂ and Fv are used consistently with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

Therefore, antibodies of the invention may be single chain antibodies or may be single domain antibodies (intrabodies or intracellular antibodies). Intrabodies are generally known in the art as single chain Fv fragments with domains of the immunoglobulin heavy (VH) and light chains (VL). Well-known functionally active antibody fragments include but are not limited to F(ab′)₂, Fab, Fv and Fd fragments of antibodies. These fragments which lack the Fc fragment of intact antibody, clear more rapidly from the circulation, and may have less non-specific tissue binding than an intact antibody (Wahl et al., J. Nuc. Med. 24:316-325 (1983)). For example, single-chain antibodies can be constructed in accordance with the methods described in U.S. Pat. No. 4,946,778 to Ladner et al. Such single-chain antibodies include the variable regions of the light and heavy chains joined by a flexible linker moiety. Methods for obtaining a single domain antibody (“Fd”) which comprises an isolated variable heavy chain single domain, also have been reported (see, for example, Ward et al., Nature 341:644-646 (1989), disclosing a method of screening to identify an antibody heavy chain variable region (V_{H} single domain antibody) with sufficient affinity for its target epitope to bind thereto in isolated form). Methods for making recombinant Fv fragments based on known antibody heavy chain and light chain variable region sequences are known in the art and have been described, e.g., Moore et al., U.S. Pat. No. 4,462,334. Other references describing the use and generation of antibody fragments include e.g., Fab fragments (Tijssen, Practice and Theory of Enzyme Immunoassays (Elsevieer, Amsterdam, 1985)), Fv fragments (Hochman et al., Biochemistry 12: 1130 (1973); Sharon et al., Biochemistry 15: 1591 (1976); Ehrilch et al., U.S. Pat. No. 4,355,023) and portions of antibody molecules (Audilore-Hargreaves, U.S. Pat. No. 4,470,925). Thus, those skilled in the art may construct antibody fragments from various portions of intact antibodies without destroying the specificity of the antibodies for the their target, e.g., pRb.

As is well-known in the art, the complementarity determining regions (CDRs) of an antibody are the portions of the antibody which are largely responsible for antibody specificity. The CDRs directly interact with the epitope of the antigen. In both the heavy chain and the light chain variable regions of IgG immunoglobulins, there are four framework regions (FR1 through FR4) separated respectively by three complementarity determining regions (CDR1 through CDR3). The framework regions (FRs) maintain the tertiary structure of the paratope, which is the portion of the antibody which is involved in the interaction with the antigen. The CDRs, and in particular the CDR3 regions, and more particularly the heavy chain CDR3 contribute to antibody specificity. Because these CDR regions and in particular the CDR3 region confer antigen specificity on the antibody these regions may be incorporated into other antibodies or peptides to confer the identical specificity onto that antibody or molecule.

The molecule useful according to the methods of the present invention may be a human antibody (e.g., a human monoclonal antibody) or an intact humanized monoclonal antibody. A “humanized monoclonal antibody” as used herein is a monoclonal antibody or functionally active fragment thereof having human constant regions and an antigen-binding region (e.g., CDR3) from a mammal of a species other than a human. Humanized monoclonal antibodies may be made by any method known in the art. Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. There are entities in the United States which will synthesize humanized antibodies from specific murine antibody regions commercially, such as Protein Design Labs (Mountain View Calif.). For instance, a humanized form of a murine anti-Rb antibody could be prepared and used according to the methods of the invention.

European Patent Application 0239400, the entire contents of which is hereby incorporated by reference, provides an exemplary teaching of the production and use of humanized monoclonal antibodies in which at least the CDR portion of a murine (or other non-human mammal) antibody is included in the humanized antibody. Briefly, the following methods are useful, as examples for constructing a humanized monoclonal antibody including at least a portion of a mouse CDR. A first replicable expression vector including a suitable promoter operably linked to a DNA sequence encoding a variable domain of an immunoglobulin (Ig) heavy or light chain and the variable domain comprising framework regions from an human antibody and a CDR region of a murine antibody is prepared. Optionally a second replicable expression vector is prepared which includes a suitable promoter operably linked to a DNA sequence encoding at least the variable domain of a complementary human Ig light or heavy chain, respectively. A cell line is then transformed with the vector(s). Preferably the cell line is an immortalized mammalian cell line of lymphoid origin, such as a myeloma, hybridoma, trioma, or quadroma cell line, or is a normal lymphoid cell which has been immortalized by transformation with a virus. The transformed cell line is then cultured under conditions known to those of skill in the art to produce the humanized antibody.

As set forth in European Patent Application 0239400 several techniques are well known in the art for creating the particular antibody domains to be inserted into the replicable vector. (Vectors and recombinant techniques are discussed in greater detail below.) For example, the DNA sequence encoding the domain may be prepared by oligonucleotide synthesis. Alternatively a synthetic gene lacking the CDR regions in which four framework regions are fused together with suitable restriction sites at the junctions, such that double stranded synthetic or restricted subcloned CDR cassettes with sticky ends could be ligated at the junctions of the framework regions. Another method involves the preparation of the DNA sequence encoding the variable CDR containing domain by oligonucleotide site-directed mutagenesis. Each of these methods is well known in the art. Therefore, those skilled in the art may construct humanized antibodies containing a murine CDR region without destroying the specificity of the antibody for its epitope.

Human monoclonal antibodies may be made by any of the methods known in the art, such as those disclosed in U.S. Pat. No. 5,567,610, issued to Borrebaeck et al., U.S. Pat. No. 5,565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, New York, 1987), and Boerner et al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

A pRb binding antibody can be used to identify pRb binding molecules. It is now routine to produce large numbers of molecules having inhibitory functions based on one or a few peptide sequences or sequence motifs. (See, e.g., Bromme, et al., Biochem. J. 315:85-89 (1996); Palmer, et al., J. Med. Chem. 38:3193-3196 (1995)). For example, an inhibitor of pRB-antibody interactions may be chosen or designed as a polypeptide or modified polypeptide having the same sequence as an Rb-binding portion of the antibody, or having structural similarity to such a sequence of the antibody. Thus, a plurality of these compounds chosen or designed may be produced, tested for inhibitory activity, and sequentially modified to optimize or alter activity, stability, and/or specificity.

The method is useful for designing a wide variety of biological mimics that are more stable than the natural counterpart, because they are typically prepared by the free radical polymerization of functional monomers, resulting in a compound with a non-biodegradable backbone. Thus, the created molecules would have the same binding properties as the anti-Rb antibody but be more stable in vivo, thus preventing Rb from interacting with components normally available in its native environment. Other methods for designing such molecules include, for example, drug design based on structure-activity relationships which require the synthesis and evaluation of a number of compounds and molecular modeling.

Binding molecules may also be identified by conventional screening methods, such as those described above. Additionally, Rb-binding molecules can be identified from combinatorial libraries. Many types of combinatorial libraries have been described. For instance, U.S. Pat. No. 5,712,171 (which describes methods for constructing arrays of synthetic molecular constructs by forming a plurality of molecular constructs having the scaffold backbone of the chemical molecule and modifying at least one location on the molecule in a logically-ordered array); U.S. Pat. No. 5,962,412 (which describes methods for making polymers having specific physiochemical properties); and U.S. Pat. No. 5,962,736 (which describes specific arrayed compounds).

By using the known anti-Rb monoclonal antibodies, it is also possible to produce anti-idiotypic antibodies which can be used to screen other antibodies to identify whether the antibody has the same binding specificity as the known monoclonal antibody. Such anti-idiotypic antibodies can be produced using well-known hybridoma techniques (Kohler and Milstein, Nature, 256:495, 1975). An anti-idiotypic antibody is an antibody which recognizes unique determinants present on the known monoclonal antibodies. These determinants are located in the hypervariable region of the antibody. It is this region which binds to a given epitope and, thus, is responsible for the specificity of the antibody. An anti-idiotypic antibody can be prepared by immunizing an animal with the known monoclonal antibodies. The immunized animal will recognize and respond to the idiotypic determinants of the immunizing known monoclonal antibodies and produce an antibody to these idiotypic determinants. By using the anti-idiotypic antibodies of the immunized animal, which are specific for the known monoclonal antibodies, it is possible to identify other clones with the same idiotype as the known monoclonal antibody used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody.

Other Rb-binding molecules of the invention can be identified using routine assays, such as binding assays. The Rb-binding molecules of the invention are isolated molecules, e.g., isolated polypeptides. As used herein, with respect to Rb-binding molecules, “isolated” means molecules are substantially pure and are essentially free of other substances with which they may be found in nature or in vivo systems to an extent practical and appropriate for their intended use. In particular, the isolated molecules are sufficiently pure and are sufficiently free from other biological constituents of their hosts cells so as to be useful in, for example, producing pharmaceutical preparations. Because an isolated molecule of the invention may be admixed with a pharmaceutically acceptable carrier in a pharmaceutical preparation, the molecule may comprise only a small percentage by weight of the preparation. The molecule is nonetheless substantially pure in that it has been substantially separated from the substances with which it may be associated in living systems. The term isolated refers to molecules which are either naturally occurring or synthetic. Thus, in some embodiments the isolated molecules are derived from natural sources. The term “isolated” as used in conjunction with the other agents provided herein, has the same meaning.

In other embodiments the isolated molecules may be synthesized or produced by recombinant means by those of skill in the art. Methods for preparing or identifying molecules which bind to a particular target are well known in the art. Molecular imprinting, for instance, may be used for the de novo construction of macromolecular structures such as peptides which bind to a particular molecule. See for example Kenneth J. Shea, Molecular Imprinting of Synthetic Network Polymers: The De Novo synthesis of Macromolecular Binding and Catalytic Sites, TRIP Vol. 2, No. 5, May 1994; Klaus Mosbach, Molecular Imprinting, Trends in Biochem. Sci., 19(9) January 1994; and Wulff, G., in Polymeric Reagents and Catalysts (Ford, W. T., Ed.) ACS Symposium Series No. 308, pp 186-230, American Chemical Society (1986). One method for preparing mimics of Rb-binding molecules involves the steps of: (i) polymerization of functional monomers around a known Rb-binding peptide that exhibits a desired activity; (ii) removal of the template molecule; and then (iii) polymerization of a second class of monomers in the void left by the template, to provide a new molecule which exhibits one or more desired properties which are similar to that of the template. In addition to preparing peptides in this manner other Rb-binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone.

Molecules which bind to Rb may also be identified by conventional screening methods such as phage display procedures (e.g., methods described in Hart, et al., J. Biol. Chem. 269:12468 (1994)). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Rb-binding molecules that bind selectively to Rb are obtained by selecting those phages which express on their surface a peptide that binds to Rb. These phages then are subjected to several cycles of reselection to identify the peptide ligand-expressing phages that have the most useful binding characteristics. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding to Rb. Alternatively, such peptide ligands can be selected from combinatorial libraries of peptides containing one or more amino acids. Such libraries can further be synthesized which contain non-peptide synthetic moieties which are less subject to enzymatic degradation compared to their naturally-occurring counterparts.

Thus, according to another aspect of the invention, a method for optimizing a selected Rb-binding molecule for its ability to bind to Rb and/or inhibit its maintenance of cell cycle arrest is provided. “Optimizing” as used herein refers to an iterative process of introducing changes to an existing system or compound and evaluating the functional significance of each change, followed by selecting the resulting system or compound associated with a functional outcome that is most improved; these steps are repeated until a desired endpoint is achieved or it appears further changes will not improve the functional outcome. The sane objective can be achieved in a parallel manner by generating a library of closely related compounds and screening the library for the compound or compounds possessing the most favorable embodiment of the characteristic being optimized. In this particular instance, optimizing a selected Rb-binding molecule for Rb-binding activity involves testing a panel of structurally related Rb-binding molecules for their ability to bind to Rb. The screening method involves contacting at least one candidate optimized Rb-binding molecule selected from a group of candidate optimized Rb-binding molecules with Rb under conditions which, in the absence of a competitor, permit a reference Rb-binding molecule to bind or remain bound to Rb. The candidate optimized Rb-binding molecule is contacted with Rb before, after, or simultaneously with contact between the labeled reference Rb-binding molecule and Rb. The residual binding of the labeled reference Rb-binding molecule to Rb is then detected. Detection of a decrease in binding of the reference Rb-binding molecule indicates that the candidate optimized Rb-binding molecule interferes with the binding of the reference Rb-binding molecule to Rb. Candidate optimized Rb-binding molecules can be generated as members of a combinatorial library of compounds, for example using SELEX technology. Gold L et al. (1995) Annu Rev Biochem 64:763:797.

This assay can involve the separation of both unbound unlabeled candidate optimized Rb-binding molecules and unbound labeled reference Rb-binding molecules from the sample. The separation step can be accomplished in any way known in the art, in a manner similar to the separation method described above. Likewise, the detection of the remaining bound labeled reference Rb-binding molecule can be accomplished in any way known in the art, in a manner similar to the detection method described above.

The screening assay can also be performed as a competition between labeled candidate optimized Rb-binding molecules (e.g., anti-Rb antibodies or fragments thereof) and unlabeled reference Rb-binding molecules. In this format, binding of the labeled optimized Rb-binding molecule to Rb is then detected. Detection of bound optimized Rb-binding molecule indicates that the candidate optimized Rb-binding molecule interferes with the binding of the reference Rb-binding molecule to Rb.

The screening assay can also be performed by contacting labeled Rb to immobilized Rb-binding molecules. In this format a panel of candidate optimized Rb-binding molecules can be presented in an array fashion on a silicon chip or in a plastic multiwell microtiter or microarray plate. Alternatively, each candidate optimized Rb-binding molecule can be separately coupled to a bead, a resin, a filter, a slide, or a biomolecular interaction analysis (BIA) chip. After contacting Rb with the immobilized candidate Rb-binding molecules and, if indicated, washing away unbound Rb, detection of complexes formed between the immobilized Rb-binding molecule and Rb provides the basis for selecting particular Rb-binding molecules as optimized.

Other assays will be apparent to those of skill in the art, having read the present specification, which are useful for determining whether a Rb-binding molecule binds to Rb and also inhibits the maintenance of cell cycle arrest.

The invention further provides detectably labeled, immobilized and conjugated forms of Rb and the molecules for use in the methods of the invention, as well as fragments and functional equivalents thereof. The Rb-binding molecules of the invention may be labeled using radiolabels, fluorescent labels, enzyme labels, free radical labels, avidin-biotin labels, or bacteriophage labels, using techniques known to the art (Chard, Laboratory Techniques in Biology, “An Introduction to Radioimmunoassay and Related Techniques,” North Holland Publishing Company (1978).

Typical fluorescent labels include fluorescein isothiocyanate, rhodamine, phycoerythrin, phycocyanin, allophycocyanin, and fluorescamine.

Typical chemiluminescent compounds include luminol, isoluminol, aromatic acridinium esters, imidazoles, and the oxalate esters.

Typical bioluminescent compounds include luciferin, and luciferase. Typical enzymes include alkaline phosphatase, β-galactosidase, glucose-6-phosphate dehydrogenase, maleate dehydrogenase, glucose oxidase, and peroxidase.

In some embodiments the Rb-binding molecule are functional equivalents of the Rb-binding molecules provided herein, e.g., anti-Rb antibodies or fragments thereof. Functional equivalents, therefore include Rb-binding polypeptides e.g., anti-Rb antibodies or fragments thereof which are different because they comprise conservative substitutions within their amino acid sequence. As used herein, “conservative substitution” refers to an amino acid substitution which does not alter the relative charge or size characteristics of the peptide in which the amino acid substitution is made. Conservative substitutions of amino acids include substitutions made amongst amino acids with the following groups: (1) M, I, L, V; (2) F, Y, W; (3) K, R, H; (4) A, G; (5) S, T; (6) Q, N; and, (7) E, D. Such substitutions can be made by a variety of methods known to one of ordinary skill in the art. For example, amino-acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene. These and other methods are known to those of ordinary skill in the art and may be found in references which compile such methods, e.g. Sambrook. et al., Molecular Cloning: A Laboratory Manual, 2nd edition, Cold Spring Harbor Laboratory Press, 1989. The activity of functionally equivalent variants of the agents of the invention can be tested by the binding and activity assays discussed herein.

The invention, in one aspect, also permits the reduction or elimination of Rb expression level or function by the construction of Rb gene “knock-outs” or “knock-downs” in cells and in animals, providing materials for treating and studying certain aspects of hearing cell damage and its regeneration. For example, a knock-out mouse (gene disruption) or a knock-down mouse (reduced gene expression by e.g., siRNA) may be constructed and examined for clinical studies of hair cell regeneration.

Hearing research has been greatly hindered by the lack of hair cell or supporting cell lines for in vitro characterization. The proliferative capacity of progenitor cells, differentiated hair cells and supporting cells provides an excellent opportunity to create such cell lines. The diverse properties of hair cells, such as vestibular and auditory hair cells, can therefore be characterized in vitro. Manipulating Rb expression level and/or function allows the sensory epithelial cells to be cultured in large numbers in vitro indefinitely (normal hair cells cannot divide and be maintained indefinitely in a culture system). It is, therefore, now possible to establish cell lines with hair cell and/or supporting cell characteristics.

Such cell lines are provided herein. The cell lines can either be established using the sensory epithelial cells in which Rb has been deleted, or using the cells in which Rb is inhibited by reducing or eliminating the expression level or function of Rb as described herein. Sensory epithelial cells, such as progenitor, supporting or hair cells, can be isolated from a subject by the disaggregation of inner ear tissue, and forming cell suspensions. Disaggregation of a tissue or a population of cells can be readily accomplished using techniques known to those skilled in the art. For example, the tissue can be disaggregated mechanically and/or treated with digestive enzymes and/or chelating agents that weaken the connections between neighboring cells, making it possible to disperse the tissue into a suspension of individual cells without appreciable cell breakage. Enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase, dispase, etc. Mechanical disruption can also be accomplished by a number of methods including, but not limited to, the use of grinders, blenders, sieves, homogenizers, pressure cells, or insonators to name but a few. For a review of tissue disaggregation techniques, see Freshney, Culture of Animal Cells, A Manual of Basic Techniques, 2d Ed., A. R. Liss, Inc., New York, 1987, Ch. 9, pp. 107-126.

Once the tissue has been reduced to a suspension of individual cells, the suspension optionally can be fractionated into subpopulations from which the desired cells can be obtained. This may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis and fluorescence-activated cell sorting. For a review of clonal selection and cell separation techniques, see Freshney, Culture of Animal Cells, A Manual of Basic Techniques, 2d Ed., A. R. Liss, Inc., New York, 1987, Ch. 11 and 12, pp. 137-168.

Cells can be cultured in an appropriate nutrient medium under conditions that are metabolically favorable for the growth of the cells. As used herein, the phrase “metabolically favorable conditions” refers to conditions that maintain cell viability. Such conditions include growth in nutrient medium at 37° C. in a 5% CO₂ incubator with greater than 90% humidity. Many commercially available media, such as RPMI 1640, Fisher's, Iscove's, McCoy's, Dulbecco's Modified Eagle's Medium, etc., and the like, which may or may not be supplemented with serum, may be suitable for use as nutrient medium. Antibiotics such as penicillin may also be included. Fungizone may also be used. In preferred embodiments, the sensory epithelial inner ear cells are cultured first, for a brief period of time in the presence of 10% serum. The medium is then changed to a serum-free medium in order to minimize the number of extraneous agents present in the medium under continuous culture. In general, these cell suspensions can be cultured according to standard cell culture techniques. In small scale, the cultures can be contained in culture plates, flasks, and dishes. In larger scale, the cultures can be contained in roller bottles, spinner flasks and other large scale culture vessels such as fermenters. Culturing in a three-dimensional, porous, solid matrix may also be used.

The cell lines cultures can be used to screen for further targets related to hair cell and/or supporting cell regeneration and protection and the compounds which act on the targets, and to characterize genes and proteins participating in the Rb pathway. Methods, therefore, are provided to study the functions of the progenitor cells, hair cells and the supporting cells. Progenitor or supporting cell lines can also be used to screen for compounds which can induce progenitor or supporting cells to become hair cells. These screening methods are, therefore, provided herein. An example of such a method comprises the steps of contacting a sample containing progenitor, supporting or hair cells with a candidate compound and determining if the candidate compound causes the desired effect (e.g., if the candidate compound causes generation, regeneration or protection of the progenitor, supporting or hair cells or if the candidate compound induces progenitor or supporting cells to become hair cells, in particular functional and differentiated hair cells.) In some embodiments, the use of explanted organ culture of the inner ear tissues are provided. The explanted culture has similar properties as the cell lines and, therefore, can be used for all the purposes mentioned for the cell lines.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., B-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the retinoblastoma (e.g., retinoblastoma inhibitory) nucleic acid molecules and genomic sequences in expression vectors, as well as to transfect host cells and cell lines, be these prokaryotic, e.g., E. coli, or eukaryotic, e.g., CHO cells, COS cells, yeast expression systems, and recombinant baculovirus expression in insect cells. Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. The expression vectors require that the pertinent sequence, i.e., those nucleic acids described supra, be operably linked to a promoter.

The invention, in one aspect, also permits the construction of retinoblatoma gene “knock-outs” and “knock-ins” in cells and in animals, providing materials for studying hair cell generation and regeneration as well as hearing damage and loss.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA or RNA encoding a retinoblastoma inhibitory nucleic acid, a mutant, fragment, or variant thereof. As used herein a “retinoblastoma inhibitory nucleic acid” is any nucleic acid that can be used to produce an agent that reduces or eliminates the expression or function of at least one retinoblastoma gene and/or protein. These nucleic acids include, for example, nucleic acids that are used to produce nucleic acids that can inhibit the transcription or translation of a retinoblastoma gene. In addition, nucleic acids that encode a protein that can inhibit retinoblastoma function are also included. For instance, the nucleic acid can encode a Rb-binding polypeptide. The heterologous DNA or RNA is placed under operable control of transcriptional elements to permit the expression of the heterologous DNA in the host cell.

Preferred systems for mRNA expression in mammalian cells are those such as pcDNA/V5-GW/D-TOPO® and pcDNA3.1 (Invitrogen) that contain a selectable marker (which facilitates the selection of stably transfected cell lines) and contain the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an eAdeno.P1A recombinant is described by Warnier et al., in intradermal injection in mice for immunization against P1A (Int. J. Cancer, 67:303-310, 1996).

The Rb-binding polypeptides of the present invention may also, of course, be produced by eukaryotic cells such as CHO cells, human hybridomas, immortalized B-lymphoblastoid cells, and the like. In this case, a vector is constructed in which eukaryotic regulatory sequences are operably joined to the nucleotide sequences encoding the peptide. The design and selection of an appropriate eukaryotic vector is within the ability and discretion of one of ordinary skill in the art. The subsequent purification of the peptides may be accomplished by any of a variety of standard means known in the art.

In another embodiment, the present invention provides host cells, both prokaryotic and eukaryotic, transformed or transfected with, and therefore including, the vectors of the present invention.

According to the methods of the invention, the compositions may be administered in a pharmaceutically acceptable composition. In general, pharmaceutically-acceptable carriers for peptides and structurally-related small molecules are well-known to those of ordinary skill in the art. As used herein, a pharmaceutically-acceptable carrier means a non-toxic material that does not interfere with the effectiveness of the biological activity of the active ingredients, i.e., the ability of an agent, such as a Rb-binding molecule, to generate or regenerate hair cells of the inner ear, preferably to restore hearing or damage. Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. The compositions of the invention may be formulated into preparations in solid, semi-solid, liquid or gaseous forms such as tablets, capsules, powders, granules, ointments, solutions, depositories, inhalants (e.g., aerosols) and injections, and usual ways for oral, parenteral or surgical administration. The invention also embraces locally administering the compositions of the invention, including as implants.

According to the methods of the invention the compositions (of the agents or cells provided herein) can be administered directly to the inner ear of a subject. In some embodiments the compositions provided herein can be administered by injection by gradual infusion over time or by any other medically acceptable mode. The administration may, for example, be intravenous, intraperitoneal, intramuscular, intracavity, subcutaneous or transdermal. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation.

The compositions of the invention are administered in therapeutically effective amounts. As used herein, an “effective amount” of the invention is a dosage which is sufficient to generate or regenerate progenitor cells, supporting cells and/or hair cells or to treat or prevent hearing damage and/or loss of balance. Preferably an effective amount of an agent is an effective amount for regenerating supporting and/or hair cells and restoring hearing or balance. Generally, a therapeutically effective amount may vary with the subject's age, condition, and sex, as well as the extent of the disease in the subject and can be determined by one of skill in the art. The dosage may be adjusted by the individual physician or veterinarian in the event of any complication. A therapeutically effective amount typically will vary from about 0.01 mg/kg to about 500 mg/kg, were typically from about 0.1 mg/kg to about 200 mg/kg, and often from about 0.2 mg/kg to about 20 mg/kg, in one or more dose administrations daily, for one or several days (depending of course of the mode of administration and the factors discussed above). For administering inner ear sensory cells, as provided herein, the dosage and administration regimen can also be determined by one of skill in the art. In general, the amount of cells that can be administered for the desired effect can be on the order of 10²-10⁴ or more cells, in one or more dose administrations. In one example, approximately 10² cells can be administered. In other examples approximately 10³ or 10⁴ cells can be administered.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Materials And Methods

Isolation of utricular sensory epithelia

Timed-pregnant CbA/CaJ mice at various gestation stages (E14.5, E15.5, E17.5, P0, P2, P6 and P12) were anesthetized with CO₂, their peritoneal cavity opened and the uterus removed and placed in cold DMEM/F12. Following isolation of embryos from the uterus, each embryo was decapitated and the utricles dissected from the temporal bone. The membranous roof of each utricle, the otoconia, and the otolithic membrane were removed (otoconia and otolithic membranes were present at E15.5, but not earlier). The utricles from each mouse were stored in RNAlater solution (Ambion, Houston, Tex.) in individual tubes at 4° C. An average of 40 utricles were used for each stage.

To separate the utricle epithelial sheets from the underlying basement membrane, utricles were incubated for one hour at 37° C. in a DMEM/F12 solution containing 500 μg/ml thermolysin (Sigma, St. Louis, Mo.). At the end of this incubation period, the utricles were transferred to an ice-cold solution of DMEM/F12/BSA without thermolysin. The sensory epithelia were delaminated from the underlying non-sensory tissue by the use of fine forceps.

Mouse utricle preparation

Semi-intact preparations of the mouse utricle were made as described previously (J. R. Holt, D. P. Corey, R. A. Eatock, J Neurosci 17, 8739 (1997); A. Rusch, R. A. Eatock, Ann NY Acad Sci 781, 71 (1996)). Temporal bones were removed from Col1A1-pRb^{+/-} and Col1A1-pRb^{-/-} embryos at E18.5. The utricles were exposed and bathed in standard extracellular solution containing 100 μg/ml protease type XXIV (Sigma, St. Louis, Mo.) for 20 minutes at room temperature to facilitate removal of the otolithic membrane. Extracellular solution contained (in mM): 144 NaCl, 0.7 NaH2PO4, 5.8 KCl, 1.3 CaCl2, 0.9 MgCl2, 5.6 D-glucose, 10 HEPES-NaOH, vitamins and minerals as in Eagle's MEM; pH 7.4, ˜320 mmol/kg.

GeneChip analysis of developing utricle

The extraction of total RNA, synthesis of cRNA, GeneChip hybridization and scanning were as described (Chen and Corey, 2002). The Microarray Analysis Suite V5 (MAS5, Affymetrix, Santa Clara, Calif.) was used to analyze the data. The data was then exported to an Excel file to be further analyzed with GeneSpring (Silicon Genetics, Redwood City, Calif.).

Using GeneSpring, MAS5 data was normalized before being further filtered by P (presence), M (marginal) and A (absence) and by fold change. Only the genes, classified as P or M, and with a 2-fold expression change in at least one of the conditions, were used for cluster analysis. The inclusion of non-ear samples such as from the heart and retina as well as cell lines served to identify the genes enriched in the utricle. Best K-mean was used to identify the number of clusters with the highest explained variability. K-mean analysis classified the genes into 15 clusters. Using the GO (Gene Ontology) classification, the cell cycle regulators were assigned to different clusters. To identify the genes primarily expressed in the sensory epithelium, RMA (robust multi-array average) (Irizarry et al., 2003) (http://www.bioconductor.org) was used to normalize all the data. The genes derived from the sensory epithelium, the stroma, or likely induced by thermolysin treatment were identified.

Analysis of pRb^{-/-} conditional mice

Rb^{loxp/loxp} mice, with the Rb1 exon 19 flanked by loxP sites (provided by Dr. Anton Berns, Netherlands Cancer Institute, Amsterdam, The Netherlands), were crossed with Rb^{loxp/+}-cre mice (Collagen1A1-cre mice (Col-cre; provided by Dr. Barbara Kream, University of Connecticut Health Center, Farmington Conn.)) (F. Liu et al., Int J Dev Biol 48, 645 (2004)) to produce Col1A1-Rb−/− embryos (pRb^{-/-} embryos). The genotyping of embryos was as described (Dr. Phil Hinds, Tufts University, Boston, Mass., personal communication and (R. Dacquin, M. Starbuck, T. Schinke, G. Karsenty, Dev Dyn 224, 245 (2002)). All animal work was conducted using procedures reviewed and approved by the institutional animal care and use committee of Massachusetts General Hospital, and were conducted in accordance with the NIH Guide for the Care and Use of Laboratory Research Animals.

BrdU labeling

Timed pregnant mice at E13.5 and E16.5 were injected with 5-bromo-2-deoxyuridine (BrdU, Sigma) (prepared in 1×PBS, pH 7.0). For E13.5 pregnant mice the injection was done once for 4 hours before the embryos were harvested, and for E16.5 pregnant mice the injection was done twice at 6-hour interval, and the embryos were harvested at E18.5. The final concentration was 50 μg BrdU per gram of body weight. The mice were sacrificed at E18.5, and the inner ear tissues were harvested and fixed with fresh 4% paraformaldehyde in PBS.

Immunohistochemistry

Frozen sections of the inner ear tissues were prepared for immunolabeling. Inner ear slides were dried for 15 minutes at 37° C. and rehydrated in 1×PBS for 5 minutes. Then the sections were subjected to an antigen unmasking treatment using the Antigen Unmasking Solution (Vector laboratories, Burlingame, Calif., Cat#h-3300), according to the protocol provided by the manufacturer. For the slides used for DAB staining, endogenous peroxidase was quenched with 1.5% H₂O₂ in methanol for 10 minutes then washed twice in 1×PBS. The blocking, primary antibody and second primary antibody incubation followed the standard protocol (Harlow and Lane, Using Antibodies: A Laboratory Manual, Cold Spring Harbor Laboratory Press, 1999). The secondary antibodies were anti-rabbit Alexa 594 and/or anti-mouse Alexa 488 for fluorescent labeling (Molecular Probes, Eugene, Oreg.) and anti-rabbit Biotinylated (Vector Laboratories, Burlingame, Calif.). Data visualization and acquisition were performed using a regular fluorescent microscope (Zeiss Axioscope 2) or a confocal microscope (Biorad, Hercules, Calif.).

Other reagents used were the following: DAPI and rhodamine-phalloidin (Molecular Probes, Eugene, Oreg.); anti-Rb (Pharmingen, San Diego, Calif.); anti-Math1 (provided by Dr. J. Johnson, University of Texas Southwestern Medical Center, Dallas, Tex.), anti-Brn-3.1 (provided by Dr. M Xiang, UMDNJ-Robert Wood Johnson Medical School, Piscataway, N.J.), anti-myo7a (Dr. T. Hasson, University of San Diego, San Diego, Calif.); anti-espin (provided by Dr. S. Heller, Massachusetts Eye and Ear Infirmary, Boston, Mass.); anti-Lhx3, -Isl-1 (DSHB, University of Iowa, Iowa City, Iowa); anti-BrdU (Accurate Chemical, Westbury, N.Y.); anti-p27kip1 (LabVision Corp., Fremont, Calif.); anti-p130 (Santa Cruz Biotech., Santa Cruz, Calif.); anti-p75ntr (Chemicon International, Temecula, Calif.); anti-Ptprq (provided by Dr. D. F. Bowen-Pope, University of Washington, Seattle, Wash.); anti-activated caspase 3 (R&D Research Systems, Minneapolis, Minn.); anti-tubulin (Sigma); anti-S100A1 (LabVision Corp.); anti-synaptophysin (provided by Dr. Jeff Macklis, Massachusetts General Hospital and Harvard Medical School, Boston Mass. 02114).

In situ analysis

Primers specific for each gene, with built-in T7 and SP6 promoter sequences, were used to amplify cDNA fragments from the inner ear cDNA pool. The PCR products were sequenced to ensure that the right genes were amplified. After purification, 1 μg of DNA template was used for making antisense and sense riboprobes, respectively, using the digoxigenin RNA labeling kit (SP6/T7) (Roche Diagnostics, Nutley, N.J.). Synthesis of the riboprobes was performed following the manufacturer's protocol.

Cryosectioned slides containing the inner ear tissues were used for in situ hybridization. The protocol for in situ hybridization was similar to what has been described previously with minor modifications (Birren et al., 1993; Tiveron et al., 1996). Briefly, thawed sections were fixed in 4% paraformaldehyde for 15 min at room temperature followed by a brief wash in PBS. The sections were then treated with Proteinase K (10 μg/ml) for 10 min followed by fixation in 4% paraformaldehyde for 15 min at room temperature. The slides were treated with 100 mM triethanolamine, pH 8.0, acetylated for 10 min at room temperature by adding dropwise acetic anhydride (0.25% final concentration) while being rocked, and washed with PBS. The slides were prehybridized for 1 hr with hybridization solution (50% formamide, 5×SSC, 1×Denhardt's, 0.1 mg/ml heparin, 0.3 mg/ml yeast RNA, 0.1% Tween 20, 5 mM EDTA) in plastic slide mailers. Hybridizations were carried out at 65° C. for 12-16 hours with the same solution in the presence of 1-2 μg/ml of probe. The slides were washed in 2×SSC at 65° C. for 15 min, then treated with 1 μg/ml RNase A at 37° C. for 30 min, followed by wash in 2×SSC and 0.2×SSC twice. The final washes included twice in 0.2×SSC for 30 min at 65° C. followed by in PBT at room temperature for 20 min.

For the labeling, slides were blocked for 1 hr at room temperature in PBT with 10% heat-inactivated sheep serum and incubated for 1.5 hr at room temperature with alkaline phosphatase-coupled anti-DIG antibody (Roche) diluted 1:2000 in PBT with 1% heat-inactivated sheep serum. The slides were washed for 5 min in AP buffer (100 mM Tris, pH 9.5, 100 mM NaCl, 50 mM MgCl2, 0.1% Tween 20) and equilibrated for 5 min in AP buffer with 5 mM Levamisol (Sigma) to block endogenous phosphatase activity. The signals were visualized by a color reaction using the NBT/BCIP reagents (NEN Life Science, Boston, Mass.). The color reaction was allowed to develop in the dark at room temperature and stopped with PBS.

FM1-43

To determine if the newly generated hair cells in the Rb^{-/-} mice were functional, the uptake of FM1-43 with fluorescent microscopy was measured after applying FM1-43 (5 μm) to the medium bathing freshly dissected E18.5 Rb^{+/-} and Rb^{-/-} utricles (E18.5 Col1A1-pRb^{+/-} and Col1A1-pRb^{-/-} utricles) for 4 minutes or 1 minute at room temperature. Dye that partitioned into the outer leaflet of the membrane was subsequently washed out with fresh bath replacements. The samples were visualized and reordered using DIC (Gale et al., 2001; Geleoc and Holt, 2003; Meyers et al., 2003) and fluorescence microscopy with identical settings for control and pRb^{-/-} utricles.

Patch Clamping

Semi-intact preparations of the mouse utricle were made as described previously (Holt et al., 1997; Rusch and Eatock, 1996). The utricles were bathed in standard extracellular solution containing 100 μg/ml protease type XXIV (Sigma, St. Louis, Mo.) for 20 minutes at room temperature (22-25° C.) to facilitate removal of the otolithic membrane.

Extracellular solution contained (in mM): 144 NaCl, 0.7 NaH₂PO₄, 5.8 KCl, 1.3 CaCl₂, 0.9 MgCl₂, 5.6 D-glucose, 10 HEPES-NaOH, vitamins and minerals as in Eagle's MEM; pH 7.4, ˜320 mmol/kg. Recording pipettes contained (in mM): 140 KCl, 0.1 CaCl₂, 10 EGTA-KOH, 3.5 MgCl₂, 2.5 Na₂ATP, 5 HEPES-KOH, 0.1 Li-GTP, 0.1 Na-cAMP; pH 7.4, ˜290 mmol/kg.

Recording pipettes were pulled from R6 glass (Garner Glass, Claremont, Calif.) and had resistances in standard solutions of 4-5 M\tilde{•}. Recording pipettes contained (in mM): 140 KCl, 0.1 CaCl2, 10 EGTA-KOH, 3.5 MgCl2, 2.5 Na2ATP, 5 HEPES-KOH, 0.1 Li-GTP, 0.1 Na-cAMP; pH 7.4, ˜290 mmol/kg. Transduction currents were recorded in the whole-cell ruptured-patch mode with an Axopatch 200B amplifier (Axon Instruments, Union City, Calif.). Hair cells were voltage clamped at −64 mV. Transduction currents were low-pass-filtered at a corner frequency, f_{c}, of 1-5 kHz (8-pole Bessel filter) and digitized >2×f_{c} with a 12-bit data acquisition board (Digidata 1200, Axon Instruments) and stored on disk. Analysis was done with Origin 6.0 (Microcal Software, Northampton, Mass.).

Transduction currents were elicited by hair bundle displacements effected with a stiff probe. Glass pipettes were pulled to a final diameter of 1-2 μm and mounted on a piezoelectric bimorph (Corey and Hudspeth, 1980). The stimulus probe was driven by voltage protocols generated with pClamp 8.0 and the Digidata 1200 and low-pass-filtered by an 8-pole Bessel filter (Model 3382, Krohn-Hite, Brockton, Mass.), with f_{c} below the probe's resonant frequency. Probe displacement as a function of applied voltage was calibrated off-line from videotaped images.

Adenovirus infection of culture utricles

Whole utricles from E17.5 and P10 flox-P-Rb mice were dissected and cultured in the medium as described (J. R. Holt et al., J Neurophysiol 81, 1881 (1999)). Hair cells are postmitotic at both stages and highly mature at P10 (A. Rusch, A. Lysakowski, R. A. Eatock, J Neurosci 18, 7487 (1998)). One day after culture, adenovirus-Cre/GFP and control adenovirus-GFP (obtained from Gene Transfer Vector Core, University of Iowa, Iowa City, Iowa) was added to the culture at a titer of 10⁸ pfu/ml, respectively. 24 hours after virus infection, the medium was replaced with fresh medium supplemented BrdU (3 μg/ml) and the cultures were continued for 9 days. The medium was changed twice during the 9-day period. The utricles were subsequently fixed and cryosectioned as described. In general adenovirus-GFP had a higher infection rate than adenovirus-Cre/GFP, and a much higher infection rate was observed for E17.5 utricles than for P10 utricles.

### EXAMPLE 1

### Identification of Cell Cycle Regulators in the Developing Utricle

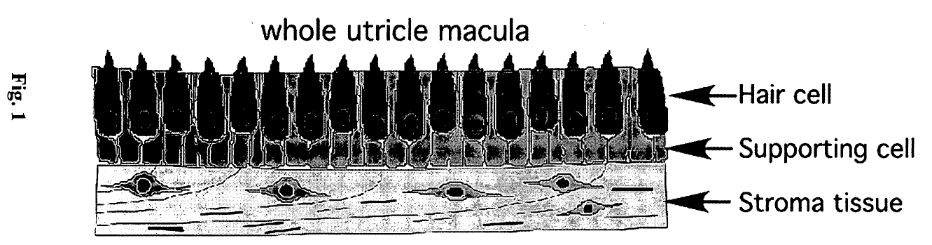

To identify cell cycle regulators with potential roles in hair cell development GeneChip analysis was used to study the gene expression patterns of the developing mouse utricle. The whole utricle is a relatively simple sensory organ, consisting of the hair cells, the supporting cells and the stromal tissues (FIG. 1). Previous studies based on the incorporation of tritiated thymidine showed that the peak of thymidine labeling occurs between E14-E15 in the utricular hair cells and supporting cells (Ruben, 1967). Therefore, developing mouse utricles at E14, E15, E17, P0, P2, P6, and P12 were collected for the GeneChip analysis. In order to understand the expression pattern in the utricular sensory epithelia cells, whole utricles from P0 and P12 mice were also treated with thermolysin, and only the sensory epithelia were isolated for GeneChip hybridization. In addition, adult mouse retina and heart samples, as well as two mouse embryonic utricular cell lines (MEU), either treated or untreated with retinoic acid, were used for the analysis (Table 1).

TABLE 1
List of samples studied

| Samples | Stages |
|---|---|
| Heart-1 | adult |
| Heart-2 | adult |
| MEU Cell | E16 |
| MEU(RA) Cell | E16 |
| Retina | adult |
| Utricle | E14 |
| Utricle | E15 |
| Utricle | E17 |
| Utricle | P0 |
| Utricle | P2 |
| Utricle + Saccule | P2 |
| Utricle | P6 |
| Utricle | P12 |
| Utricle Sensory Epithelium | P0 |
| Utricle Sensory Epithelium | P12 |

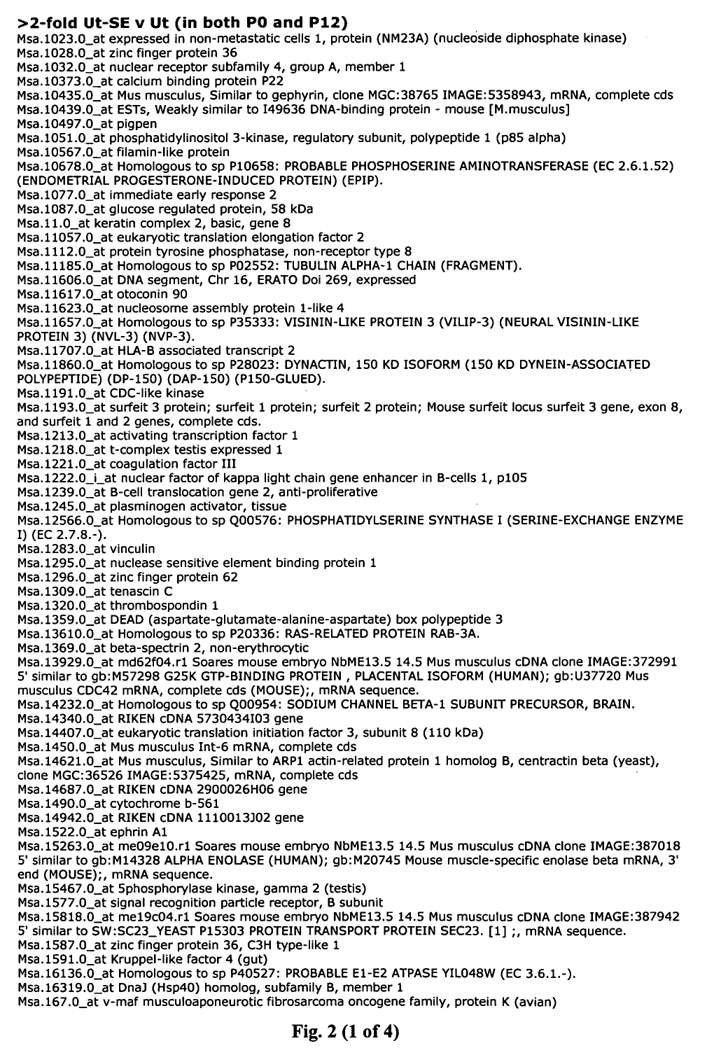

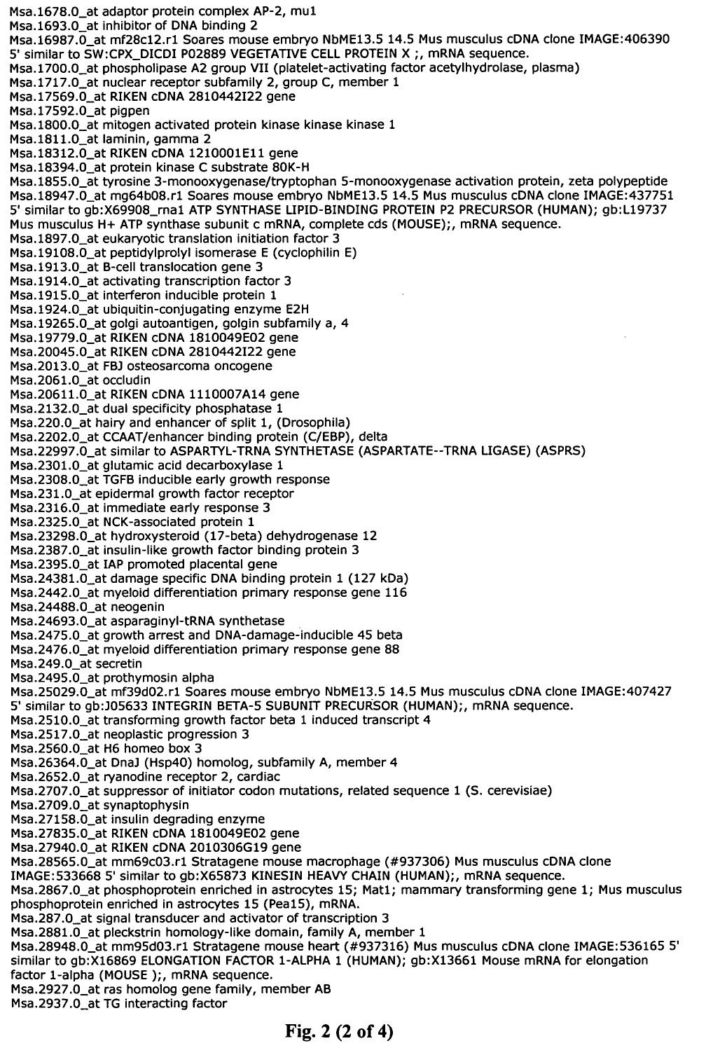

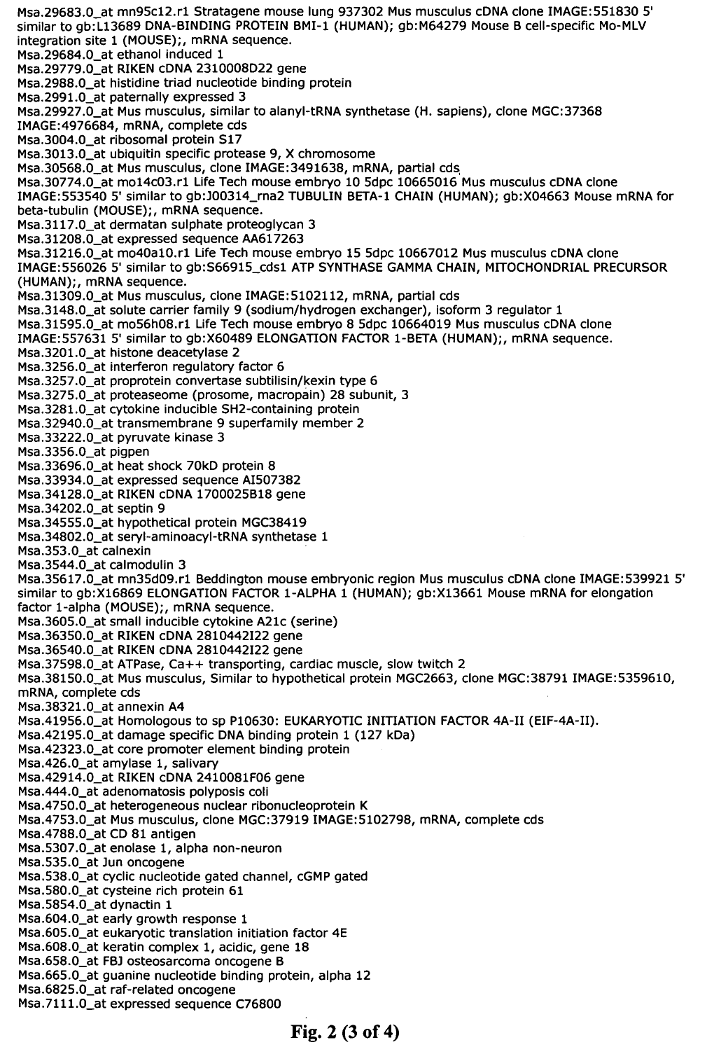

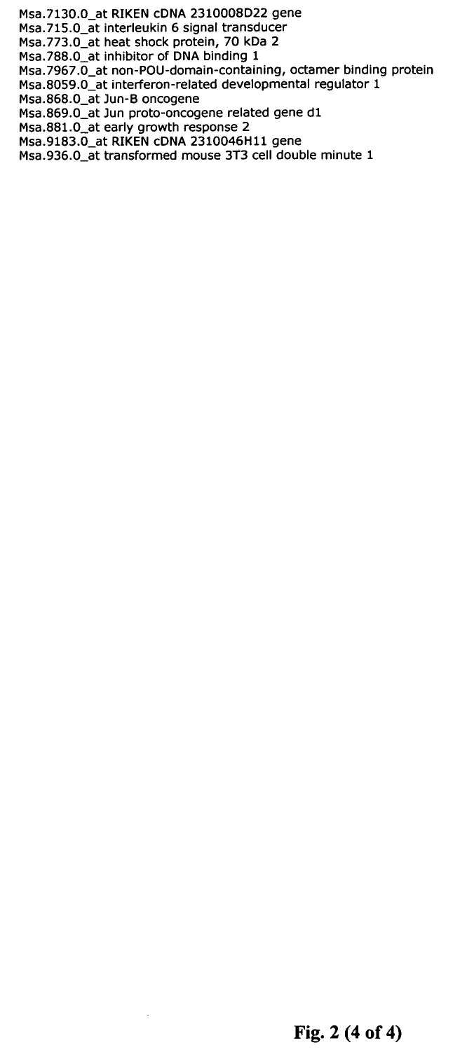

The murine 6.5k chipset with ˜6500 genes and ESTs was used for hybridization. The data were first generated using MAS5 and then were further analyzed with K-mean cluster using GeneSpring (Silicon Genetics) to identify the genes with coregulation during development. RMA was used to compare the expression of the thermolysin treated samples to the whole utricles, and the genes primarily expressed in the sensory epithelia (for instance myosin VI and Math1) and the stroma (Norrie gene and peripheral myelin protein) were identified (FIG. 2). A number of genes were upregulated in the thermolysin treated samples when compared to the whole utricles at comparable stage, including Math1. These genes are primarily those whose expression was induced by thermolysin treatment.

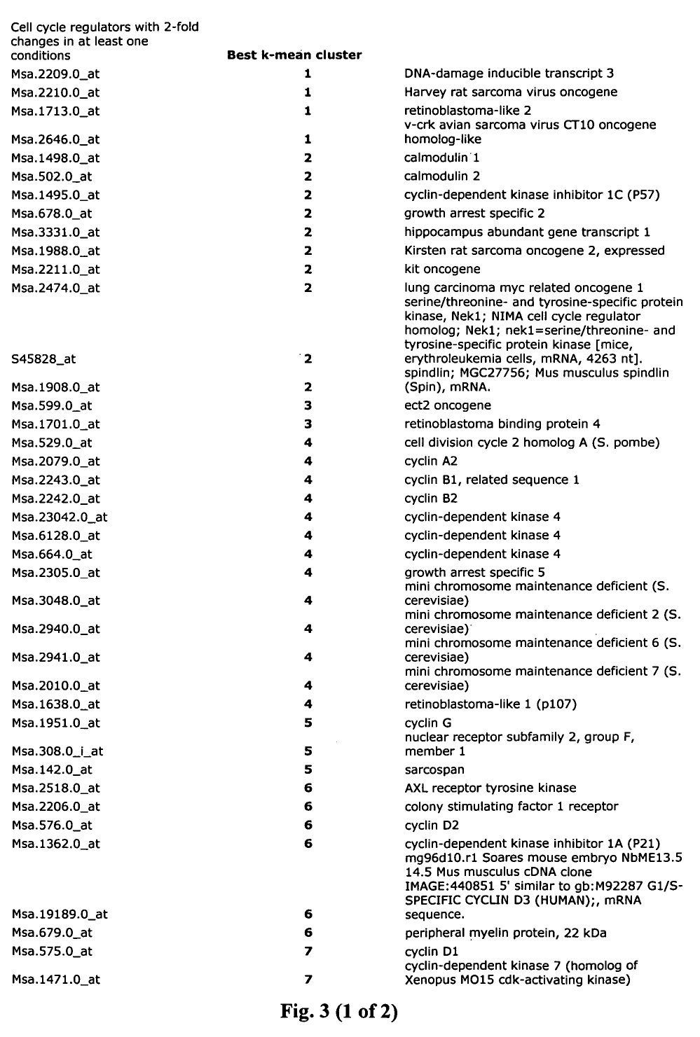

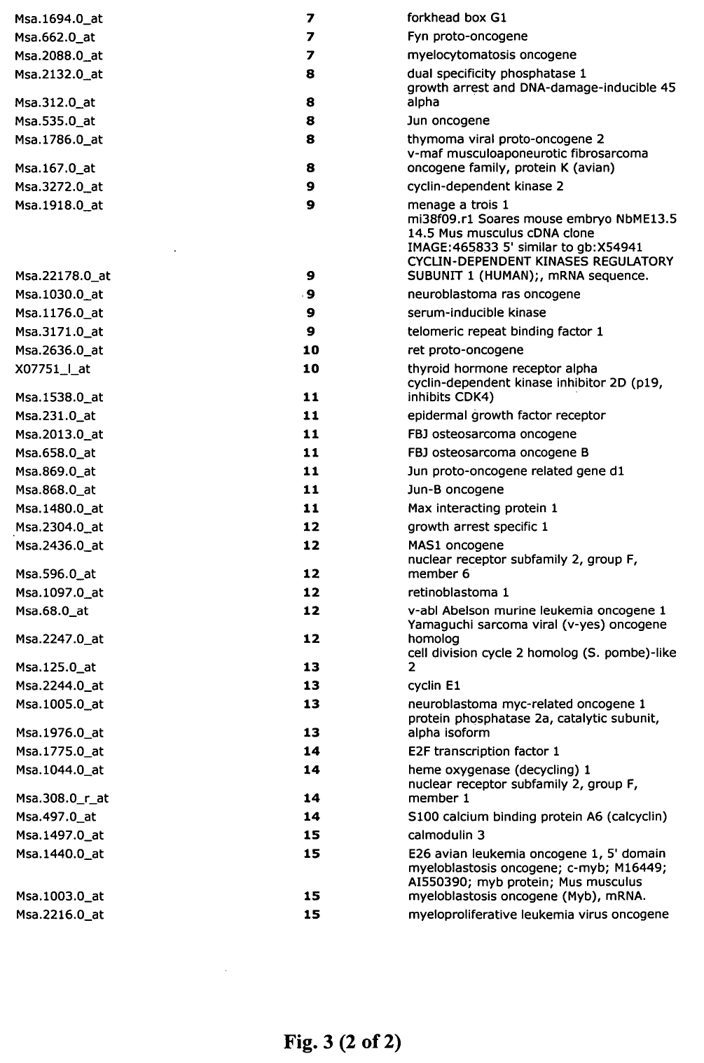

Using GO classification all the cell cycle regulators (144 in total) that passed the data filtering were first identified. 81 cell cycle regulators were identified (i.e. the genes expressed in at least one of the samples with an expression level that changed over 2-fold in at least one of the conditions). By K-mean analysis they were clustered into 15 groups (FIG. 3). The early development of the utricle is dominated by proliferation that is evident by the high level of expression of many cell cycle genes, including 9 cyclin genes from E14 through E17 (FIG. 4 Panel A, clusters 4, 7 and 9). Of particular interest was the negative cell growth control genes derived from the sensory epithelial cells, as they may be involved in cell cycle exit and the establishment of the postmitotic status of the sensory precursors. This process is prerequisite for the terminal differentiation of the hair cells during development, as no hair cells in S phase have been labeled with hair cell markers.

Many negative cell growth regulators were identified in clusters 11 and 12. Cluster 11 included many genes that are upregulated during development such as Max interacting protein 1 (Mxi1) and Cdk4 inhibitor p19 (p19^{ink4d}), whereas cluster 12 contained the genes with consistent expression throughout development including retinoblastoma and growth arrest specific protein 1 (gas1). In total, 13 genes known to be negative cell cycle regulators are in the two clusters (Table 1), and with the exception of p19^{ink4d}, most of these genes have not been characterized in the inner ear. Taken together this group of cell cycle regulator genes represents the potential candidates in controlling growth arrest in the developing utricle.

To provide further evidence to show that the genes from the thermolysin treated samples were indeed of sensory epithelia origin, the expression of a selected group of the cell cycle regulators were studied in the inner ear using in situ hybridization. All the genes tested, including Jun-B, Abelson murine leukemia oncogene (Abl1) and Max interacting protein Mxi1, were expressed in the sensory epithelium, in particular in hair cells (FIG. 4, Panels C-E). Therefore, GeneChip analysis proved to be a powerful tool that can be used to identify cell cycle regulators in the developing inner ear.

The retinoblastoma gene (Rb) is expressed in the sensory epithelial cells of the mouse inner ear

Of the negative cell growth genes the retinoblastoma family members showed interesting expression patterns: Rb1 maintained a constant level of expression throughout development, Rbl1 (p107) showed downregulation whereas Rbl2 (p130) exhibited upregulation during development (FIG. 4, Panel B). The expression patterns also indicated that they were primarily derived from the sensory epithelia cells, which is supported by their expression in the thermolysin treated samples.

Using both GeneChip and immunohistochemistry, the retinoblastoma gene (Rb) was detected in the sensory epithelial cells including the hair cells and the supporting cells, in the developing mouse inner ear. The GeneChip analysis of the expression of the Rb family members in the developing utricle correlates with the current understanding of their roles during the cell cycle, i.e. the level of p107 is highest between G1-S phase and the p130 level is highest during G0-G1 phase (FIG. 4, Panel B) (Classon and Dyson, 2001). Collectively, the results suggest that the Rb family members, individually or in combination, may be involved in cell cycle control in the utricle epithelium. To understand their normal expression patterns the distributions of pRb and p130 (Rbl2) were studied in the developing mouse inner ear, with anti-Rb and anti-p130 antibodies.

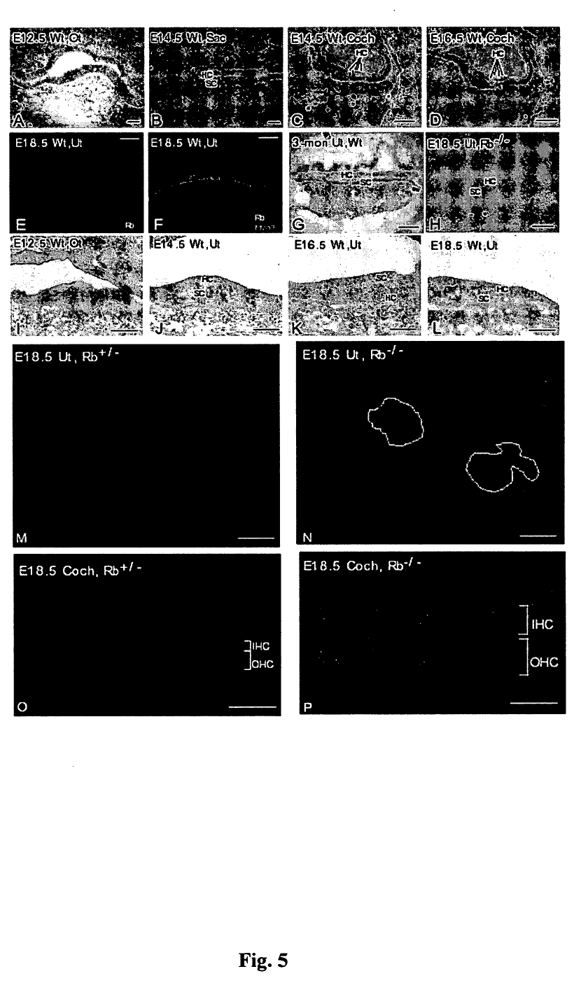

Using immunostaining, pRb was detected at an early stage of development. At E12.5 the expression of pRb was detected ubiquitously in the otocyst at a moderate level (FIG. 5, Panel A). In the saccule at E14.5, pRb showed a differential expression pattern with a hair cell prominent pattern of expression, which persists in the E18.5 utricle (FIG. 5, Panels E-F), and less expression in the supporting cells (FIG. 5, Panel B). A similar pattern persists throughout the rest of embryonic development such that by E18.5, pRb is predominantly expressed in the utricular hair cells with further reduced expression in the supporting cells (FIG. 5, Panels E-F). pRb expression is also maintained in the hair cells and the supporting cells of the adult utricle (FIG. 5, Panel G), indicating that it may be required for the life of the sensory epithelial cells. Similarly, the expression of pRb in the cochlea showed prominent hair cell expression during embryonic stages, with moderate expression in the supporting cells (FIG. 5, Panels C and D). The same pattern can be seen in the P6 mouse cochlea. Immunohistochemical study with anti-p130 antibodies also confirmed the expression pattern observed with the GeneChip analysis. Little p130 expression was detected during early development of the inner ear (FIG. 5 Panels I-J), whereas expression was upregulated in the hair cells during later development, with weak expression in the supporting cells (FIG. 5, Panels K-L).

The expression pattern of the Rb family members, therefore, strongly suggested their potential roles in inner ear development, in particular the sensory epithelial cells. As a first step to comprehensively characterizing their inner ear functions, conditional Rb knockout mice were studied.

Rb is known to suppress the function of the E2f family, especially E2f1-E2f3. p130 and p107 are thought to participate in the Rb pathway (Classon and Dyson, 2001). The expression of p130 or E2f5 is not altered in the pRb^{-/-} inner ear sensory cells. It has also been shown that in general p107 will be upregulated when pRb is deleted, to compensate for pRb function (Berns, 2003). Given the distinct expression pattern of p107 during utricle development it is likely that p107 will participate in a compensatory mechanism after loss of Rb. It is unlikely, however, that any change in p107 expression is adequate to compensate for the loss of pRb function, as demonstrated by continuous hair cell and supporting cell division.

Rb conditional knockout mice

Since germline Rb1 knockout mice are embryonic lethal between E13-E15 when most of the hair cells are not fully developed (Jacks, 1992), the conditional Rb knockout model provided by Dr. Phil Hinds (Tufts University, Boston, Mass.) was used for this study. Mice with loxP sites flanking exon 19 of the Rb1 gene were obtained as were mice with cre-recombinase under the control of collagen 1A1 promoter (Dr. Barbara Kream, University of Connecticut Health Center, Farmington, Conn.). The Rb1^{loxp/loxp} mice were crossed with the Col-cre mice (Dacquin et al., 2002) to create pRb^{-/-} mice. The 3.6 kb Col1A1 promoter driving cre results in the expression of cre-recombinase in a pattern similar to endogenous Col1A1 expression. The pRb conditional knockout mice produced from this cross were perinatal lethal, due to respiratory failure (G. Gutierrez and P. Hinds, unpublished observations), and therefore, the analysis focused on mouse embryos.

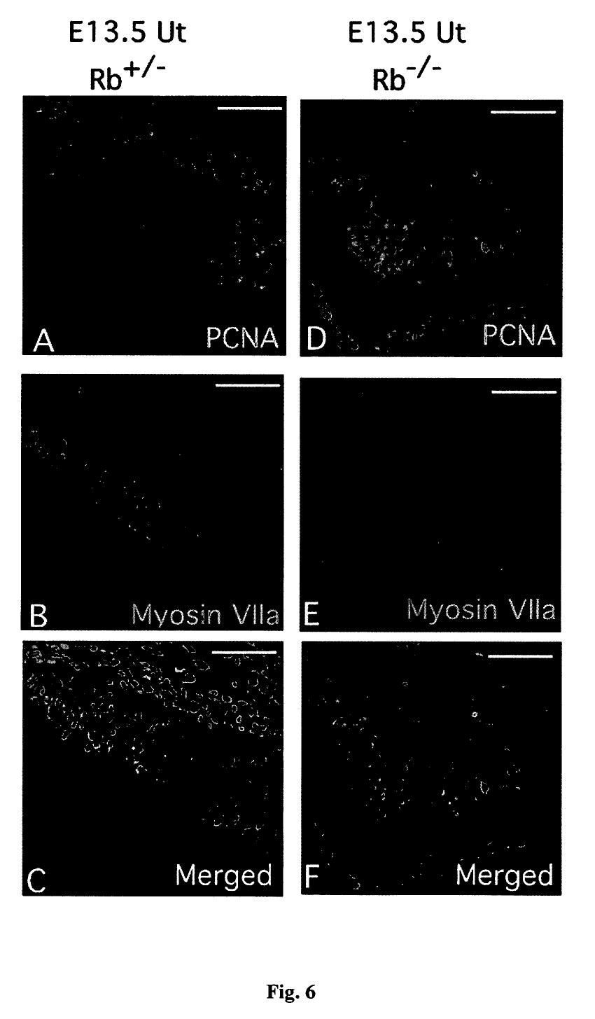

The expression of collagen1A1 (Col1A1) in the developing utricle was examined. GeneChip analysis showed that Col1A1 is expressed in the utricle, with the main expression being derived from the stroma. In situ hybridization confirmed the expression of Col1A1 in the developing otic placode as early as E10.5 and, subsequently, in the hair cells and supporting cells (FIG. 6), whereas prominent expression was found in the stromal tissue. These results indicate that Rb is likely to be deleted at an early stage, when Col1A1 promoter activates the expression of cre-recombinase at E10.5.

The expression of pRb in the pRb^{-/-} mice was studied using the anti-Rb antibody. pRb was found to be completely absent in the sensory epithelial cells in both the pRb^{-/-} inner ear at E13.5 (the earliest stage examined) and E18.5 (FIG. 5, Panel H). Therefore, the early activity of cre-recombinase under Col1A1 promoter was sufficient to completely eliminate the production of pRb in the inner ear pRb^{-/-} mice.

Rb controls cell cycle exit in the hair cells and to a lesser extent in the supporting cells

a) Increased hair cell number in the pRb^{-/-} mouse inner ear

If pRb is critical in regulating the cell cycle exit control during hair cell development, loss of pRb could allow hair cells to remain in the cell cycle, thereby producing more hair cells. Using confocal microscopy the number of utricular hair cells of pRb^{-/-} mice at E18.5 was counted, after rhodamine-phalloidin staining of the hair bundles. The hair bundles were clearly labeled in the pRb^{-/-} utricle, which exhibited normal morphology, as in the controls. The overall hair bundle morphology of the knockout and control mice was similar, judging from confocal microscopy. Compared to the control littermates there was a 40% increase in the number of hair bundles in the pRb^{-/-} mice: pRb^{-/-}, 1406±73 (mean±SD), n=3; pRb^{+/-}, 987±62 (mean±SD), n=5; P<0.05. It was evident from the confocal study that the distribution of the hair cells in the pRb^{-/-} mice was abnormal (FIG. 5, Panels M and N). Instead of the regular mosaic pattern of hair cells surrounded by supporting cells (as in the controls), clustered hair cells were observed in some regions whereas fewer hair cells were seen in other regions in the pRb^{-/-} utricles, suggesting that the mechanisms maintaining the normal cell pattern was disrupted. A more drastic increase in the number of hair cells was observed in the pRb^{-/-} cochlea. In the E18.5 pRb^{-/-} cochlea, instead of the normal one row of inner hair cells and three rows of outer hair cells, there were as many as 3-4 rows of inner hair cells and 7-8 rows of outer hair cells (FIG. 5, Panels O and P), consistent with the increase in the number of hair cells in the pRb^{-/-} utricle. Close examination showed that most of the pRb^{-/-} cochlea hair cells have hair bundles, but the orientation of the bundles was irregular (FIG. 5, Panel P).

b) The hair cells in the pRb^{-/-} mice undergo cell division

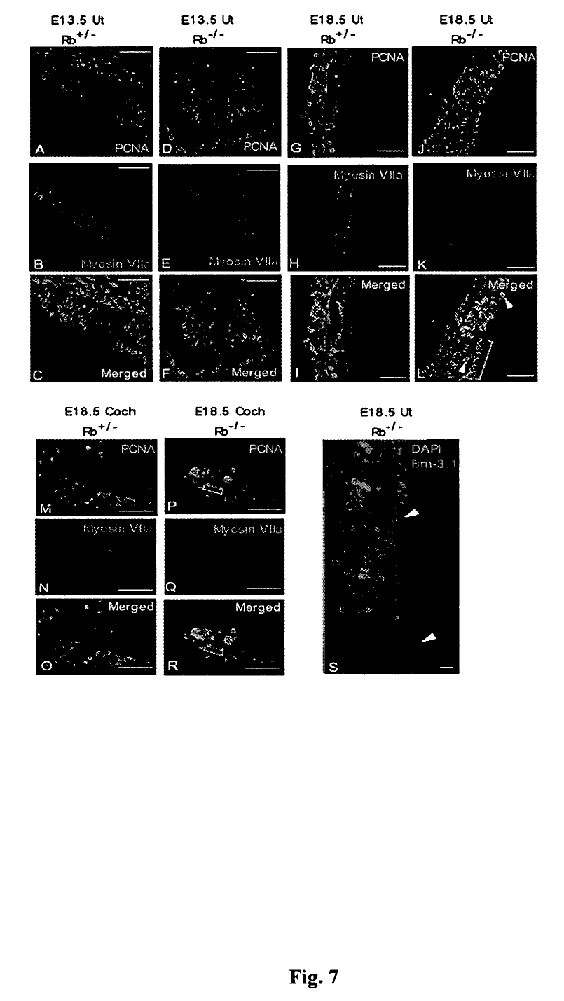

To investigate if the differentiated hair cells can still enter the cell cycle, anti-PCNA antibody was used to localize the dividing cells together with the hair cell marker myosin VIIa (myo7a). During normal development the progenitor cells differentiate to hair cells after they become postmitotic. At E13.5 in the pRb^{-/-} utricle, double-labeling showed that all of the hair cells stained strongly for PCNA, whereas the control hair cells were PCNA negative (FIG. 7, Panels A-F). Also, the supporting cells beneath the hair cells in the pRb^{-/-} utricle stained more intensely for PCNA than the control, suggesting higher cell cycle activity. More strikingly, most of the highly differentiated pRb^{-/-} utricular hair cells at E18.5 were prominently stained by PCNA, whereas there were no PCNA labeled hair cells in the control utricle. In comparison, the utricular supporting cells in both the knockout and control mice had some PCNA labeling (FIG. 7, Panels G-L). In the pRb^{-/-} mice, the continuous cell division of hair cells led to vastly increased hair cell numbers. The sensory epithelium of a normal utricle has a well defined structure with one row of hair cell nuclei on top of one row of supporting cell nuclei. However, in the pRb^{-/-} utricle, there were as many as 3-4 rows of hair cells, and the hair cell nuclei were also found in the supporting cell nuclei layer (FIG. 7, Panels K and L). In addition, the hair cells, located along the apical surface of the pRb^{-/-} utricular sensory epithelium, were also labeled with PCNA, indicating that most of the apical hair cells were still undergoing cell cycling (bracket in FIG. 7, Panel L).

At E18.5 most of the pRb^{-/-} cochlear hair cells stained intensely for PCNA, similar to the labeling in the pRb^{-/-} utricle hair cells. No labeling was observed in the control cochlear hair cells. The large number of hair cells in the pRb^{-/-} cochlea also confirmed the observations made in the confocal study. In addition, some of the supporting cells in the pRb^{-/-} cochlea were also stained strongly for PCNA, yet in the control, PCNA labeling in the supporting cells was completely absent (FIG. 7, Panels M-R). Therefore, in addition to its prominent role in the hair cells, pRb is also involved in the control of cell cycle arrest in the supporting cells in the cochlea.

In further support of the observation of cycling hair cells, dividing hair cells in the M phase were observed in the pRb^{-/-} utricle (FIG. 7, Panel S). In a double-labeling experiment using DAPI and a hair specific transcription factor Brn-3.1 (for nuclear staining), the majority of the hair cells showed overlapping DAPI and Brn-3.1 labeling. However, in the hair cells in the M phase of the cell cycle, the Brn-3.1 labeling was separated from the DAPI labeling. The DAPI labeled condensed chromosomes were seen undergoing the process of segregating into two daughter nuclei. Therefore, the results demonstrated that the differentiated inner ear hair cells can re-enter and complete the cell cycle, as a consequence of the loss of pRb function. pRb, therefore, is a key regulator involved in the maintenance of the postmitotic status of the differentiated hair cells.

The proliferation of the hair cells was further studied by injecting BrdU into pregnant mice at E16.5 twice at 6-hour intervals. The embryos were then harvested at E18.5, 48 hours after initial injection. During normal development, at E16.5 the hair cells are postmitotic and have started to express many hair cell marker genes. This was clearly demonstrated in the control pRb^{+/-} mice since there were no BrdU labeled hair cells (FIG. 9, Panels B and H). Also there were no BrdU positive cochlear supporting cells in the control pRb^{+/-} mice (FIG. 9, Panel B), whereas in the utricle some supporting cells were BrdU positive (FIG. 9, Panel B H), consistent with previous studies (Ruben, 1967). In the pRb^{-/-} mice, both the cochlea and the vestibular hair cells were labeled with BrdU (FIG. 9, Panels E and K), as were the supporting cells (FIG. 9, Panels E and K). Comparing the labeling intensities between the hair cells and the supporting cells in the pRb^{-/-} mice, the labeling in the hair cells was much weaker than in the supporting cells, indicating that the hair cells had undergone further cell division after initial incorporation of BrdU. No labeling was detected in the control hair cells or cochlear supporting cells. To further quantify the BrdU labeling, in both the pRb^{-/-} cochlea and vestibular system, the number of intensely labeled hair cells (with signals roughly equal to that in the most intensely labeled supporting cells) and weakly labeled hair cells (with signals about the half or less than the intensely labeled cells) was determined. It was found that 83% (251/300) of the labeled hair cells had weak BrdU labeling, while in the supporting cells, 58% (125/174) were classified as having weak BrdU labeling. In fact, the labeling in most hair cells was less than 1/4 of that in the intensely labeled cells (FIG. 9, Panel K). Therefore, within 48 hours of initial BrdU injection 83% or most of the labeled hair cells had undergone more than one round of cell cycling, consistent with continuous hair cell division. The BrdU labeling study also indicated that the rate of cell cycling in the supporting cells is lower than in the hair cells in the pRb^{-/-} mice. These results indicate that Rb plays a key role in the cell cycle arrest of hair cells.

Although some pRb^{-/-} hair cells were not labeled with BrdU, it is likely that these hair cells are dividing hair cells in the S-phase, outside the time frame when BrdU was available, i.e. the first 12 of 48 hours. This was supported by the PCNA labeling experiment where a majority of the E18.5 hair cells were PCNA positive, indicating that given a longer time frame most of hair cells would be labeled as PCNA measures the activity between G1-S phases of the cell cycle (Woods et al., 1991). Therefore, it is likely that all the hair cells in the pRb^{-/-} mice were actively dividing.

c) p27kip1 and p57kip2 are likely to be involved in the pRb pathway

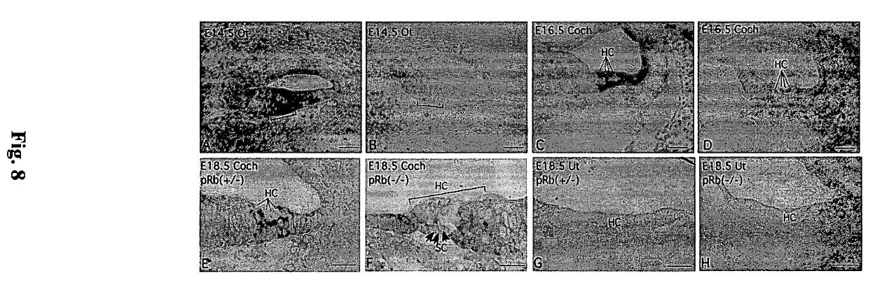

To further understand how the supporting cells re-enter the cell cycle in the pRb^{-/-} cochlea, the expression of p27kip1 was studied, a Cdk4 kinase inhibitor with prominent expression in the cochlear supporting cells. Downregulation of p27kip1 in the cochlea hair cell precursors correlated with upregulation of pRb in the same cells, during early development (FIG. 8, Panels A-D). This implies that for a progenitor cell to become a hair cell a switch in expression pattern between p27kip1 and Rb may be required, thereby maintaining the postmitotic status of the hair cell. In the p27kip1 mice the cochlear progenitor cells re-enter the cell cycle, and subsequently give rise to additional hair cells and supporting cells in the cochlea (Chen and Segil, 1999; Lowenheim et al., 1999). However, only 2 rows of the inner and 4 rows of the outer hair cells were generated in the adult p27kip1 cochlea, indicating that the lack of p27kip1 is not sufficient to drive the differentiated hair cells into cell cycle.

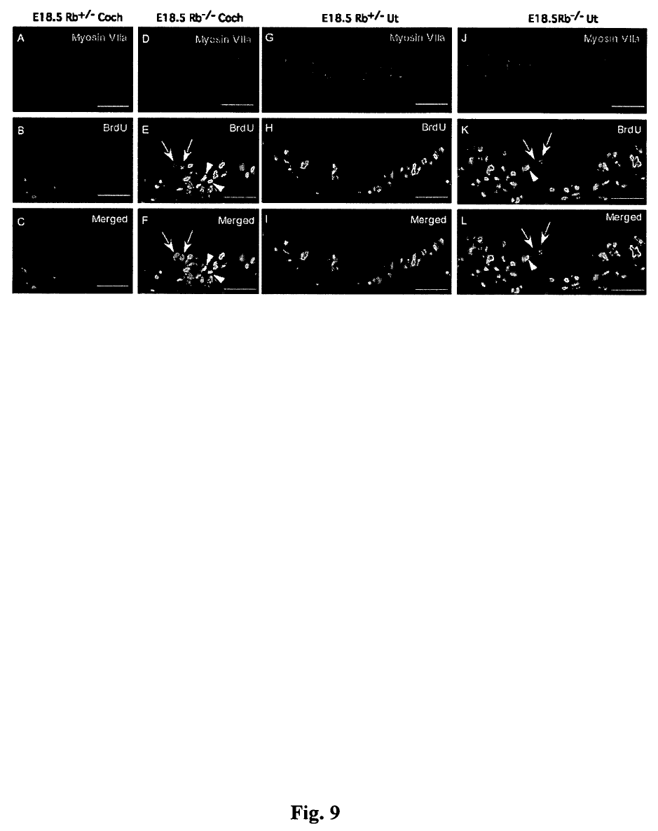

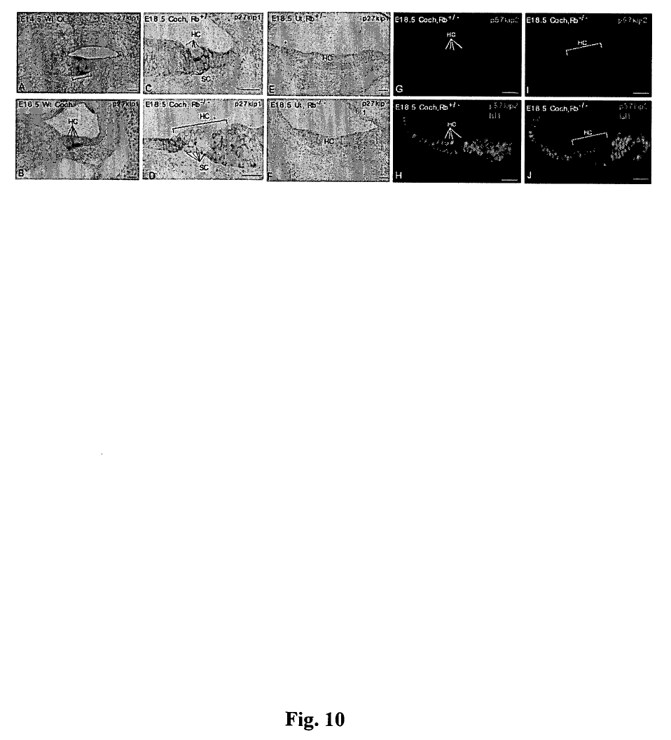

Loss of p27kip1 leads to proliferation of the cochlear supporting cells, suggesting that both pRb and p27kip1 control the postmitotic status of the supporting cells. Interestingly, there is a general reduction of p27kip1 expression in the pRb^{-/-} supporting cells, and in some supporting cells p27kip1 is completely absent (FIG. 10, Panels E and F). This indicates a possible genetic interaction between pRb and p27kip1 in the cochlear supporting cells. This is in agreement with a previous study that indicated the possible interaction between Rb and p27kip1 in controlling cell cycle exit, although the underlying mechanism is not clear (Alexander, 2001). The observations described herein support such an interaction. The cell division of the pRb^{-/-} cochlear supporting cells may thus be a consequence of the downregulation of p27kip1, in addition to the loss of pRb. In addition, the expression of p27kip1 in the utricle supporting cells was examined and residual expression of p27kip1 was observed (FIG. 10, Panels G and H). This is in agreement with the GeneChip analysis that classified p27kip1 as absent throughout utricle development. Hence p27kip1 is unlikely to be the main gene maintaining the postmitotic status of the utricle supporting cells.

At E14 when p27kip1 has established ZNPC within the sensory patch of the primordial organ of Corti, the Rb expression is maintained in both the sensory and non-sensory cells. It is therefore likely that the exit of the cell cycle of the sensory precursor cells may be initiated primarily by p27kip1, which occurs prior to the differentiation of the hair cells. This is supported by studies which showed that upregulation of p27kip1 and cell cycle withdrawal proceed the differentiation of neurons (Farah, 2000). Immediately after the initiation of hair cell differentiation, Rb is upregulated in the hair cell whose p27kip1 is downregulated. Therefore there appears to be a coordinated expression control between p27kip1 and Rb, to ensure that the cell cycle arrest is maintained in the differentiating hair cells.

During normal development the sensory precursor cells become postmitotic prior to differentiation which involves patterning of hair cells and supporting cells. This study showed that loss of Rb leads to re-entry to the cell cycle of primarily the hair cells, and the massive increase in hair cell number clearly disrupted the regular hair cell/supporting cell pattern. Therefore the correct number of postmitotic cells is largely maintained by Rb in the cochlea, together with p27kip1. The particular role of pRb in the maintenance of postmitotic hair cells helps to explain the observation that in the p27kip1 mouse cochlea only one additional row of inner and outer hair cells is produced. It is possible that deletion of p27kip1 leads to increased number of hair cell precursors, and differentiation of the precursor cells to the hair cells results in the upregulation of the Rb gene, thereby blocking the differentiated hair cells from re-entering the cell cycle. In the cochlear supporting cells however, both pRb and p27kip1 are expressed and loss of function for either one can lead to cell cycle re-entry. Therefore unlike the hair cells where the function of pRb is likely to replace that of p27kip1, functions of both molecules are required for the maintenance of quiescent supporting cells. However, the rate of cell division in the pRb^{-/-} supporting cells is considerably lower than that in the hair cells. This is likely due to the presence of p27kip1 in the supporting cells, thereby partially compensating the function of pRb. The compensation by p27kip1 may not be complete, however, as the re-entry of cell cycle and downregulation of p27kip1 are observed in some supporting cells. There is, therefore, likely a genetic mechanism, which is different from that in the hair cells, by which the functions of p27kip1 and pRb are coordinated in the supporting cells.

To further test if some other negative cell cycle regulators, identified through GeneChip analysis, are involved in the Rb controlled pathway, the expression patterns of p57kip2, Mxi1, Abl1 and Jun-B were studied. Immunostaining with anti-p57kip2 antibody showed that at E18.5 p57kip2 was normally expressed in the outer hair cells in the cochlea, and in some of the utricular hair cells. However p57kip2 was markedly reduced in the pRb^{-/-} cochlear hair cells (FIG. 10, Panels G-J), while in the utricle it was completely absent. Therefore, p57kip2 may participate in the pRb pathway, to maintain the postmitotic state of some hair cells. In situ analysis of Mxi1, Abl1 and Jun-B showed no change in the expression in the pRb^{-/-} inner ear, indicating that they are not part of the pRb pathway.

d) The newly derived hair cells are differentiated and functional

The Rb gene is involved in the differentiation of different cell types. For example Rb is required for the differentiation of the osteoblast by physically interacting and activating osteoblast transcription factor CBFA1 (Thomas, 2001). Increasing evidence has indicated that pRb has a role in neural development, is required for cell cycle exit in some neurons, and is required for differentiation in other neurons (Ferguson, 2002; Marino, 2003). The observation of cell division of hair cells in the pRb^{-/-} mice at E18.5 indicated that the genetic program for the hair cell differentiation was intact in the pRb^{-/-} hair cells, and that pRb primarily functions as a suppressor of cell cycle re-entry.

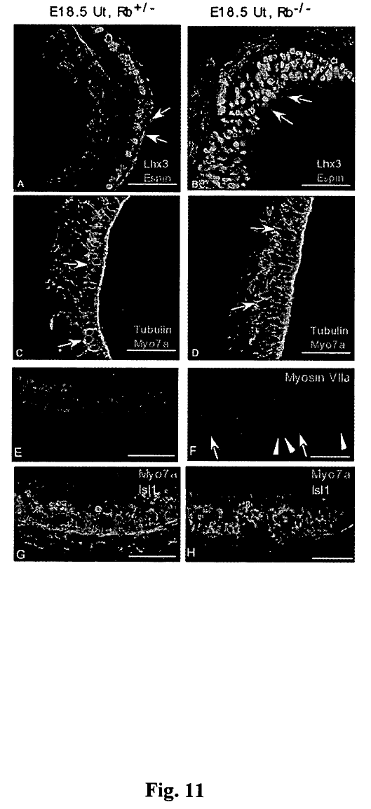

Only the hair cells on the apical surface of the pRb^{-/-} utricle appeared to have normal structure. The stereocilia were intact, and the cells bore the normal pear-like appearance of hair cells. Also, the hair bundles were labeled with actin-cross linking protein, espin (FIG. 11, Panels A and B). However, the rest of the hair cells located below the apical region did not have hair bundles (observed by the lack of espin or phalloidin labeling), were of variable shape (instead of being round many nuclei were cylindrical) and some were seen facing away from the lumen (FIG. 11, Panels D-F). These abnormalities may reflect newly generated hair cells failing to migrate longitudinally. The utricular hair bundle count in the confocal study, therefore, severely underestimated the increase in the number of hair cells in the pRb^{-/-} mice, as it missed the hair cells underneath those located along the apical region. Instead they increased the thickness of the sensory epithelium. For the apical hair cells in the M phase, the hair bundles were missing. Similar results were observed for all other vestibular organs including the saccule and the crista. However, unlike in the utricle, the extra pRb^{-/-} cochlear hair cells were still located along the apical region and had a typical hair cell shape and extended along the organ of Corti laterally (FIG. 7, Panel R and FIG. 9, Panel F). In addition to myosin VIIa and espin staining, the hair cells in the pRb^{-/-} mice were also labeled with other hair cell specific markers including Math1, Brn-3.1, Lhx3, calretinin and parvalbumin-3, demonstrating that they were indeed differentiated hair cells.

Generally, hair cells form synapses with axons of ganglion neurons, which can be labeled with acetylated tubulin. Therefore, the tubulin distribution in the pRb^{-/-} utricle was studied. Indeed labeling with anti-tubulin antibody showed, similar to the controls, that most of the hair cells in the pRb^{-/-} mice were surrounded by nerve fibers with tubulin labeling (FIG. 11, Panels C and D). This indicated that the newly generated hair cells can attract axons to form synapses. In contrast to the control hair cells, there was a disorganization of nerve fibers due to the abnormal distribution of the hair cells in the pRb^{-/-} mice. In addition, staining by synaptophysin (which labels the nerve terminals) showed membrane labeling around most of the pRb^{-/-} hair cells, similar to the control. The data provided herein suggest that the differentiation of hair cells does not require the function of pRb and is, therefore, pRb independent.

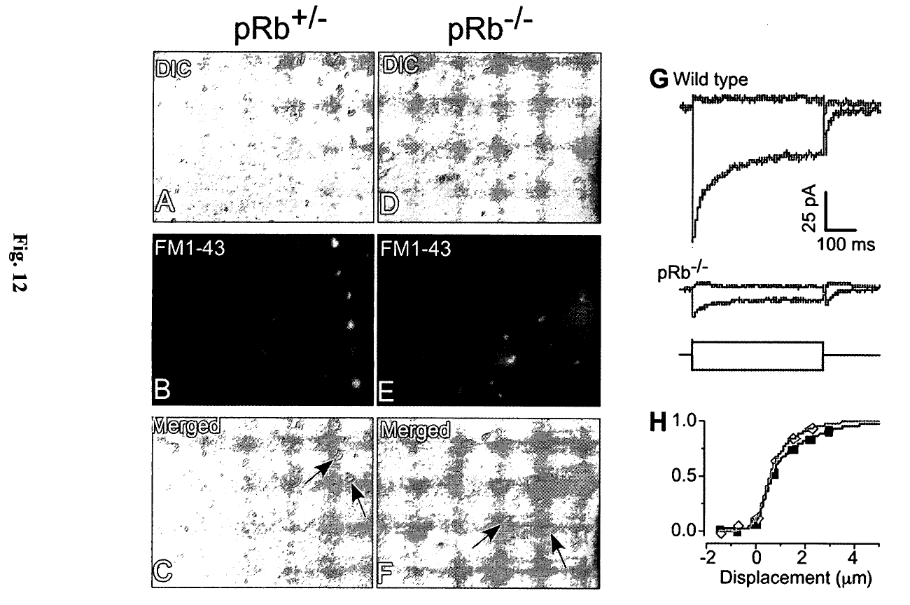

In addition, recent studies have shown that the hair cells can take up FM1-43, a small styryl dye, through the open mechanotransduction channel (Gale, 2001; Geleoc, 2003; Meyers, 2003). The uptake of FM1-43 is an indication of the presence of the functional mechanotransduction channels, a hallmark for a functional hair cell. To determine if the newly generated hair cells in the pRb^{-/-} mice were functional, the uptake of FM1-43 was measured. DIC was used to visualize the hair bundles. FIG. 12 shows the FM 1-43 uptake in both the pRb^{+/-} and pRb^{-/-} utricles associated with the cells with hair bundles in both samples. Therefore, most of the hair cells in the pRb^{-/-} mice are likely to be functional as determined by FM1-43 uptake.

To further illustrate that the transduction apparatus in the pRb^{-/-} hair cells are functional, patch clamping was performed to measure transduction currents of the utricular hair cells. The results showed that transduction currents were elicited in both pRb^{-/-} and pRb^{+/-} hair cells (FIG. 12 Panels G and H), with smaller response in the pRb^{-/-} hair cells. This is likely to be the consequence of the fact that cycling hair cells can only form the bundles outside the M phase, thereby producing the bundles that are relatively immature. These results demonstrated that the cycling pRb^{-/-} hair cells are functional.

Deletion of the pRb in the hair cell did not seem to elicit an abnormal differentiation process, judging by the labeling of various hair cell markers, the overall normal structure of the stereocilia and the FM1-43 uptake through functional channels. In fact, all the hair cell markers tested were expressed by the Rb^{-/-} hair cells, similar to the controls. The overall architecture of the hair cells is relatively normal with properly formed stereocilia. The assay with dye FM1-43 uptake experiment also showed the newly derived hair cells were functional, in agreement with a recent study which demonstrated that between embryonic days E16-E17 the inner ear hair cells have acquired the necessary components for the assembly of functional transduction channels (Geleoc, 2003). The study showed that the differentiation and proliferation processes appear to be decoupled in the pRb^{-/-} hair cells, and the differentiation of the hair cells is, therefore, likely pRb independent.

e) There is no evidence of apoptosis in the sensory epithelial cells of the pRb^{-/-} inner ear

Germline Rb^{-/-} mice die between E13-E15 with hematopoietic and neurological defects. In the CNS in germline pRb^{-/-} mice, there is extensive apoptosis as the result of aberrant entry of S phase in differentiating cells (Slack, 1998; Gloster, 1999). During normal development apoptosis has been observed in the spiral ganglion cells and the greater epithelial ridge (GER, located around the inner hair cells), in C3H/HeJ mice (Kamiya, 2001). Since one of the main functions of pRb during development is to protect cells from apoptosis whether programmed cell death occurred in pRb^{-/-} inner ear differentiated hair cells, after re-entry into the cell cycle was examined. Generally, when hair cells die they are extruded through the apical surface of the sensory epithelium, leaving a temporary space in the lumen. The examination of the utricles at all stages (multiple samples from E13.5 and E18.5) did not identify such an event, suggesting that pRb is not involved in the cell death pathway during normal development.

Cell death was further studied with anti-active caspase3 antibody in the pRb^{-/-} utricle and the cochlea. Significant labeling was not observed. However, in a positive control, the Brn-3.1 knockout mouse cochlea showed numerous caspase3 positive cells. Altogether, the data show that the absence of pRb does not lead to increased cell death in the inner ear. The hair cell apoptotic pathway in development is therefore pRb independent. Therefore, it is possible to manipulate the expression, level or function of pRb in hair cells and supporting cells to induce their re-entry into the cell cycle. The newly generated hair cells thus manipulated are fully differentiated and functional and do not undergo apoptosis. It is possible, therefore, to regenerate differentiated and functional hair cells in the mammalian (e.g., in the human) inner ear.

Different molecules and mechanisms are likely involved in hair cell apoptosis, such as p19ink4d during development and MAPK-JNK signal pathway in stress induced cell death (Chen, 2003; Wang, 2003). Inhibition of MAPK-JNK pathway protects the hair cells from ototoxic drugs and acoustic trauma induced cell death. Interestingly a recent study showed that Rb physically interacts with c-Jun NH(2)-terminal kinase/stress-activated protein kinase (JNK/SAPK), a member of the mammalian MAPK family, thereby inhibiting JNK/SAPK mediated apoptotic cell death induced by ultraviolet radiation or cytotoxic effect of topoisomerase I inhibitor camptothecin (Shim, 2000; Lauricella, 2001). pRb may have an additional protective role in preventing trauma induced hair cell death.

Isl-1 is downregulated in the Rb^{-/-} hair cells

In a separate study, two homeodomain transcription factors of the LIM family, Lim-3 transcription factor (Lhx3) and Isl-1, both with inner ear sensory cell expression patterns were identified and characterized. In the inner ear it was found that Lhx3 had hair cell specific expression starting at E13.5, whereas Isl-1 showed expression in the otocyst as early as E11.5. The expression of Lhx3 remained hair cell specific while Isl-1 expression remained predominantly in the supporting cells, with downregulated expression in the hair cells. The timing of expression of Lhx3 is very similar to that of myosin VIIa, indicating that it can be used as a relatively late hair cell marker (compared to Math1 or Brn-3.1 expression), whereas Isl-1 can be used to primarily label the supporting cells. Both Lhx3 and Isl1 maintain their expression in the adult sensory epithelial cells.

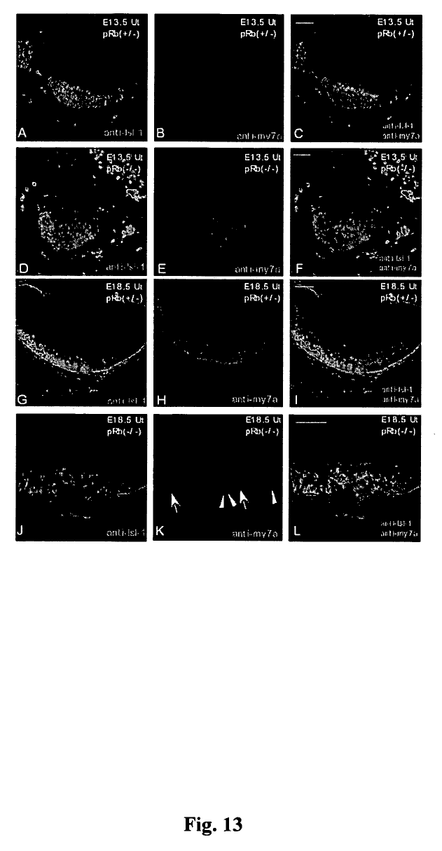

In the E13.5 utricle, Isl-1 labeled 3-4 layers of supporting cells in the pRb^{-/-} and control mice. In the hair cells, there was little Isl-1 labeling in the pRb^{-/-} samples but strong labeling in the control (FIG. 13, Panels A-F). The downregulation of Isl-1 in the pRb^{-/-} hair cells indicates that Isl-1 may be downstream of Rb. In the E13.5 utricle, the number and position of the supporting cells were very similar in the pRb^{-/-} and control mice. In the E18.5 pRb^{-/-} utricle, Isl-1 labeling was exclusively in the supporting cells (FIG. 13, Panel J), in contrast to both the supporting cell and the hair cell labeling in the control (FIG. 13, Panel G), again suggesting that Isl-1 is downstream of Rb and its downregulation may be required for hair cell production. In addition, in the E18.5 pRb^{-/-} utricle, many hair cells were in the space that is normally occupied by the supporting cells, while some supporting cells were in the mid-region of the sensory epithelium. This indicates that some of the supporting cells might have been displaced from their basal locations by the newly produced hair cells (FIG. 13, Panels J-L).

Evidence that the supporting cells are induced to become hair cells

Many pRb^{-/-} hair cell nuclei appeared along the base of the utricle epithelium, among the supporting cells. This indicates that these cells may either be newly generated hair cells migrating from the apical region or supporting cells induced to become hair cells, or both.

In the normal utricle, the hair cells and the supporting cells have very different nuclear shapes; the hair cell nucleus is round (from Brn-3.1 and Lhx3 staining) and the supporting cell nucleus is cylindrical (Isl-1 staining). Some pRb^{-/-} hair cells in the base of the utricle have the same cylindrical nuclei as the supporting cells, whereas the hair cells above the basal lumen have, in general, a round shape (FIG. 13, Panel K). Some hair cells located within the supporting cells zone also have very weak myo7a expression, suggesting that they are likely new hair cells derived from the supporting cells.

The induction of the supporting cell to hair cell is also supported by the expression of the hair cell markers that recapitulates the sequential expression patterns during development. During normal inner ear development the presumptive hair cells will first express the bHLH transcription factor Math1 at E12.5. Math1 expression is followed by the expression of another transcription factor Brn-3.1. Subsequently the hair cells express other hair cell markers such as myosin VIIa and Lhx3 at E14.5 in the cochlea. If, the supporting cells in the pRb^{-/-} utricle are to become hair cells, they are expected to express Math1 and Brn-3.1, before they express myosin VIIa or Lhx3.

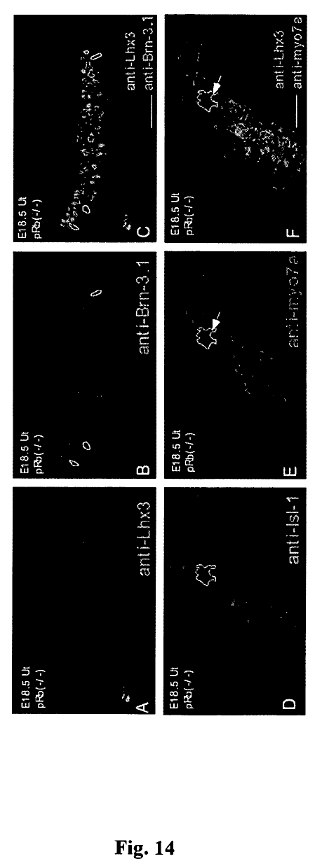

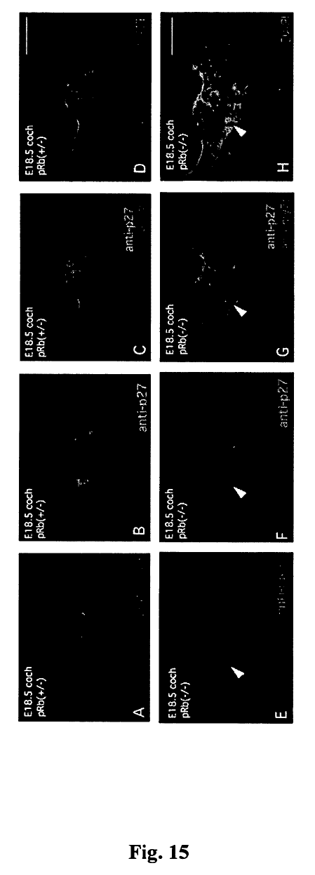

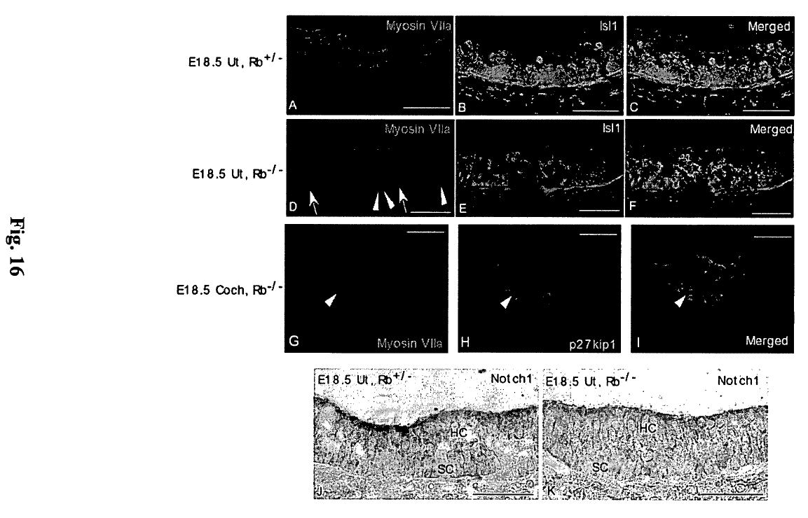

The E18.5 pRb^{-/-} utricle was studied using Brn-3.1 and Lhx3 double-labeling (during normal hair cell development Brn-3.1 is expressed earlier than Lhx3). While the majority of the hair cells were double-labeled with both Brn-3.1 and Lhx3, some hair cells were only labeled with Brn-3.1 and not Lhx3 (FIG. 14, Panels A-C), indicating that they were newly formed hair cells. In contrast, no hair cells were labeled with Lhx3 and not Brn-3.1. In another experiment, using adjacent sections stained with myosin VIIa (myo7a) and Brn-3.1 antibodies (anti-rabbit), similar to the previous experiment, Brn-3.1 labeled some hair cells which were myo7a negative. However, every myo7a positive hair cell was also Brn-3.1 positive. In most cases, when only Brn-3.1 expression was detected, the signal was weak, signaling the beginning of Brn-3.1 expression. A similar expression pattern was also observed for Math1 and Lhx3 labeling. In the E18.5 control utricle, all the hair cells were double-labeled with Brn-3.1 and Lhx3, indicating that they were fully differentiated hair cells. Isl-1 and myo7a labeling of the E18.5 Rb^{-/-} utricle showed that most of the sensory epithelial cells in the pRb^{-/-} utricle were labeled with either Isl1 (supporting cell) or myo7a (hair cell). However, it was routinely observed that some cells within the supporting cell zone were both Isl-1 and myo7a negative. It was evident from their location and the cylindrical shape of their nuclei that they were primarily the supporting cells. However, individual cells within the region were seen to express myosin VIIa at a low level, indicating that they may be new hair cells derived from the supporting cells (FIG. 14, Panels D-F). This also suggests that other supporting cells with downregulation of Isl-1 might be in transition to becoming hair cells. This is consistent with the observation that Isl-1 is absent in the pRb-^{-/-} hair cells in the utricle. The combined results suggest that the supporting cells in the pRb^{-/-} utricle were induced to transdifferentiate into hair cells.

The expression of hair cell marker in the Rb^{-/-} cochlear supporting cell zone

The possibility that the supporting cells can be induced to become hair cells in the E18.5 pRb^{-/-} cochlea was further investigated. As has been shown previously, the initiation of hair cell differentiation coincides with the downregulation of p27kip1. By E16.5, p27kip1 is only expressed in the cochlear supporting cells (FIG. 8, Panels A and C). In the E18.5 pRb^{-/-} organ of Corti it was observed that p27kip1 was downregulated in some cells within the supporting cells zone, suggesting that these cells were in transition to becoming hair cells (FIG. 8, Panels E and F). Indeed some p27kip1 negative cells in the supporting cell zone also started to express low levels of myosin VIIa, suggesting again that they are likely to be the newly differentiated hair cells (FIG. 15, Panels G-I). Taken together the results suggest that p27kip1 is downregulated in pRb^{-/-} cochlea supporting cells, which then become hair cells. Therefore, the studies in both the vestibule and the cochlea in the pRb^{-/-} mice provided support for the induction of the supporting cells to hair cells.

Notch pathway may play a role in supporting cell to hair cell induction

Notch pathway plays a major role in generating the mosaic patterns of the hair cell and supporting cell, through interactions between Notch1 and its ligands (Kiernan et al., 2001; Lanford et al., 1999; Zine and de Ribaupierre, 2002; Zine et al., 2000). Previous studies have shown that perturbation of Notch pathway, either in vivo or in vitro (Zhang, 2000; Zine, 2000; Lanford, 1999), can produce supernumerary hair cells via dedifferentiation of the supporting cells.

To determine if the Notch pathway plays any role in the supporting cell to hair cell induction in the pRb^{-/-} inner ear, the expression of the major components in the Notch pathway including Notch1, jag1, jag2, delta1 and numb using in situ hybridization was studied. The analysis showed a marked reduction in Notch1 expression in the supporting cells of pRb^{-/-} mice, whereas the expression of the ligands was largely unaffected. Since Rb is expressed in normal supporting cells, the deletion of the Rb gene in the pRb^{-/-} supporting cells likely resulted in the reduced expression of Notch1, potentiating them to become hair cells. Therefore disruption of the Notch pathway may have contributed to the supporting cell to hair cell induction in the pRb^{-/-} mice, although it is not clear if the reduction of Notch1 is directly the result of Rb deletion in the supporting cells or is a consequence of overproduction of the neighboring hair cells. This question can be better addressed using the conditional Rb^{-/-} mice with Rb being specifically abolished in the hair cell (for example with Brn-3.1-Cre mice). Such a model should provide a clear view of supporting cell to hair cell conversion, as a continuous induction process should deplete or severely reduce the number of the supporting cells in the postnatal mice.

### EXAMPLE 2

In fish, amphibians and birds, regeneration of sensory hair cells through asymmetric cell divisions of supporting cells can contribute to recovery of hearing and balance after hair cell loss caused by trauma or toxicity (J. T. Corwin, D. A. Cotanche, Science 240, 1772 (1988); B. M. Ryals, E. W. Rubel, Science 240, 1774 (1988)). Mammalian hair cells do not spontaneously regenerate, even though supporting cells in vestibular sensory epithelia retain a limited ability to divide (A. Forge, L. Li, J. T. Corwin, G. Nevill, Science 259, 1616 (1993); M. E. Warchol, P. R. Lambert, B. J. Goldstein, A. Forge, J. T. Corwin, Science 259, 1619 (1993)). Consequently, hair cell death in mammals often leads to permanent hearing and balance impairment.

As the inner ear develops, hair cell progenitor cells exit from the cell cycle and, like neurons, terminally differentiate. Negative cell-cycle regulators apparently maintain the postmitotic status of hair cells and contribute to their terminal differentiation. Cyclin-dependent-kinase inhibitors, p27Kip1 and p19Ink4d, participate in cell-cycle exit of hair cell progenitors and in hair cell apoptosis, respectively (H. Lowenheim et al., Proc Natl Acad Sci USA 96, 4084 (1999); P. Chen et al., Nat Cell Biol 5, 422 (2003)). However the key regulators of cell-cycle exit and concomitant hair cell terminal differentiation remain elusive. The retinoblastoma protein, pRb, encoded by the retinoblastoma gene, Rb1, functions in cell-cycle exit, differentiation and survival (M. Classon, E. Harlow, Nat Rev Cancer 2, 910 (2002); M. M. Lipinski, T. Jacks, Oncogene 18, 7873 (1999)). pRb is a member of the pocket protein family, which includes p107 (encoded by Rbl1, BC069179) and p130 (encoded by Rbl2, BC020528). Like pRb, p107 and p130 cause cell-cycle arrest when overexpressed (M. Classon, N. Dyson, Exp Cell Res 264, 135 (2001)). Germline pRb^{-/-} animals die in utero around E13.5, with severe defects in lens development, hematopoiesis, myogenesis, osteogenesis and neurogenesis (M. Classon, E. Harlow, Nat Rev Cancer 2, 910 (2002); T. Jacks et al., Nature 359, 295 (1992); K. L. Ferguson, R. S. Slack, Neuroreport 12, A55 (2001); D. M. Thomas et al., Mol Cell 8, 303 (2001)). In both the central and peripheral nervous systems, neurons undergo ectopic mitoses and subsequent apoptosis (K. L. Ferguson, R. S. Slack, Neuroreport 12, A55 (2001), R. S. Slack, H. El-Bizri, J. Wong, D. J. Belliveau, F. D. Miller, J Cell Biol 140, 1497 (1998)). Mice with Rb1 conditionally deleted in the central nervous system show an increase in neuronal number due to aberrant S-phase entry, without apoptosis (K. L. Ferguson et al., Embo J 21, 3337 (2002); D. MacPherson et al., Mol Cell Biol 23, 1044 (2003); S. Marino, D. Hoogervoorst, S. Brandner, A. Berns, Development 130, 3359 (2003)). However, it is not clear whether these supernumerary neurons are highly differentiated or functional.

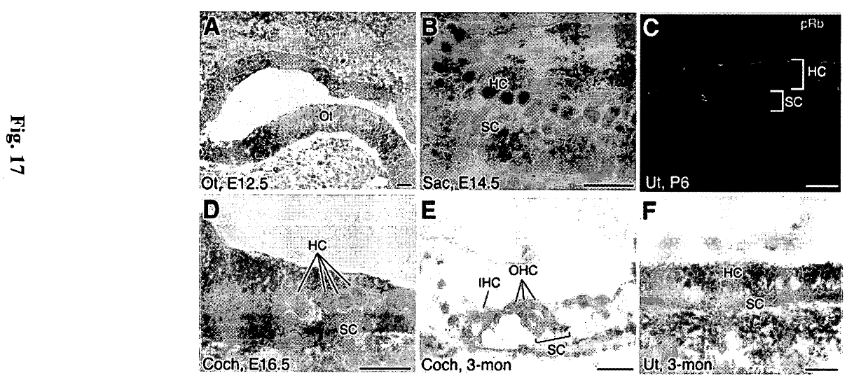

To identify molecules involved in cell-cycle regulation during hair cell development oligonucleotide microarrays were used to study gene expression in the developing mouse utricle, a balance organ of the inner ear. It was noticed that retinoblastoma family members show a suggestive pattern: from E14.5 to P12, Rb1 expression was constant, Rbl1 showed downregulation, and Rbl2 exhibited upregulation. An anti-pRb antibody weakly labeled all cells in the E12.5 otocyst (FIG. 17, Panel A), and labeling was prominent in all hair cells from embryo to adult (FIG. 17, Panels B-F). Therefore, pRb was thought to play a role in suppressing cell division in hair cells.

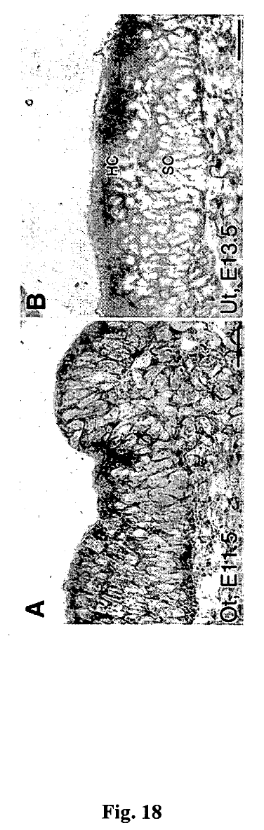

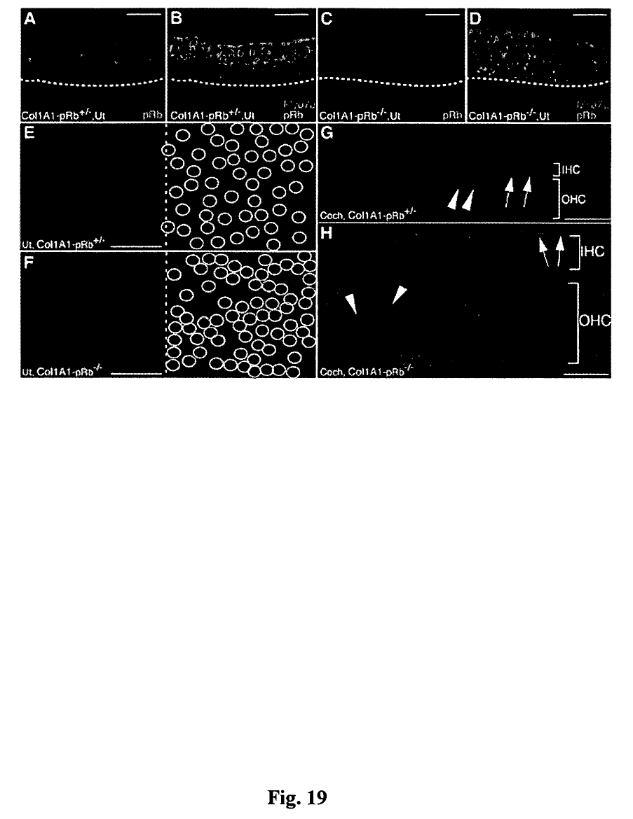

Because germline pRb^{-/-} mice die around E13.5 (T. Jacks et al., Nature 359, 295 (1992)), when hair cells are extremely immature, a conditional pRb knockout was studied. Mice with lox-P sites flanking exon 19 of the Rb1 gene (Rbl1oxp) (M. Vooijs, H. te Riele, M. van der Valk, A. Berns, Oncogene 21, 4635 (2002)) were crossed with mice carrying cre under the control of the 3.6 kb collagen1A1 (Col1A1) promoter, which express cre-recombinase in a pattern similar to endogenous Col1A1 (F. Liu et al., Int J Dev Biol 48, 645 (2004)). Because these pRb conditional knockout mice (Col1A1-pRb^{-/-}) die perinatally, embryos were studied. By in situ hybridization, Col1A1 was detected ubiquitously in the E11.5 otocyst, but later reduced in hair cells and supporting cells (FIG. 18). In Col1A1-pRb^{-/-} inner ears, pRb was undetectable in the sensory epithelium (FIG. 19, Panels C and D).

Hair bundle numbers were increased in pRb^{-/-} utricles and cochleas

Cells with hair bundles in E18.5 Col1A1-pRb^{-/-} utricles were counted. Col1A1-pRb^{-/-} utricles had 40% more cells with bundles than littermate controls (Col1A1-pRb^{-/-}: 1406±73 (mean±SD), N=3; Col1A1-pRb^{-/-}: 987±62, N=5; P<0.05) (FIG. 19, Panels E and F). A more dramatic increase in hair bundle number was observed in cochleas. While littermate controls had one row of inner hair cells and three rows of outer hair cells, Col1A1-pRb^{-/-} cochleas had 3-4 rows of inner hair cells and 7-8 rows of outer hair cells. Most Col1A1-pRb^{-/-} cochlear hair cells had bundles, but many were not properly oriented (FIG. 19, Panels G and H). The increase in hair cell number in Col1A1-pRb^{-/-} ears suggested that new hair cells arose through an increase in differentiation-competent progenitor cells and/or through continuing hair cell division.

Loss of pRb leads to proliferation and differentiation

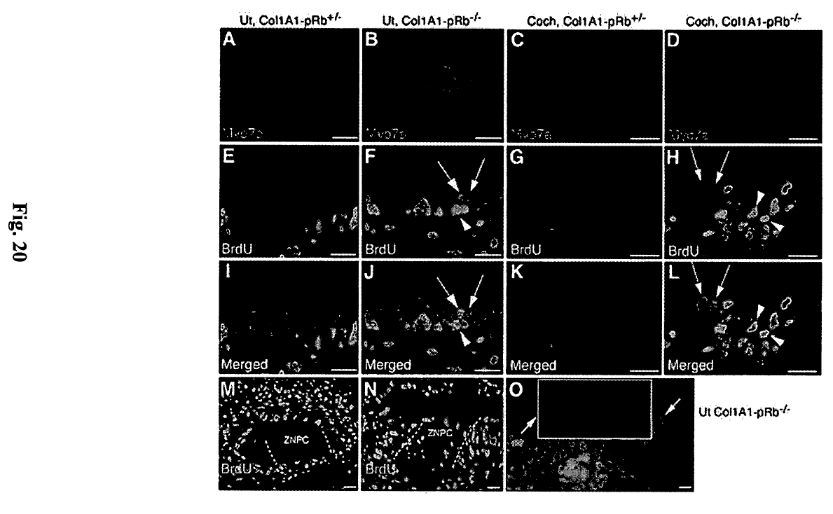

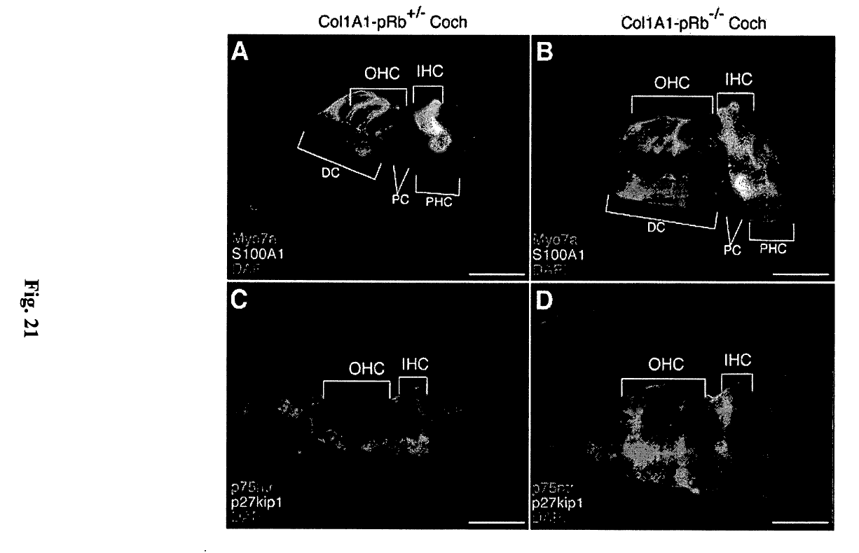

To test hair-cell proliferation specifically, E16.5 pregnant mothers were injected with BrdU and embryos harvested at E18.5. During normal development, mouse hair cells become postmitotic as early as E12.5 (M. Xiang, W. Q. Gao, T. Hasson, J. J. Shin, Development 125, 3935 (1998)). As expected, no hair cells or cochlear supporting cells were BrdU-positive in control mice (FIG. 20, Panels I and K). In contrast, many hair cells and cochlear supporting cells were BrdU-positive in Col1A1-pRb^{-/-} mice, indicating that they had entered S-phase (FIG. 20, Panels J and L). BrdU labeling in hair cells tended to be weaker than in supporting cells, suggesting hair cells had further divided, diluting the BrdU (84% of hair cells were weakly labeled vs. 58% of supporting cells, with “weak” considered less than half the level of the brightest supporting cells). An increased ratio of outer hair cells to Deiters' cells was also observed, suggesting continuous hair-cell division (Col1A1-pRb^{-/-}: OHC/DC:1.45±0.057, mean±SD, N=51; Col1A1-pRb^{-/-}/:0.79±0.037, N=22; P<0.0001). The proliferation of Col1A1-pRb^{-/-} cochlear supporting cells appeared to be cell-specific (FIG. 21), as more Deiters' cells were observed than in controls (S100A1 labeling) but not more Pillar cells (p75ntr labeling).

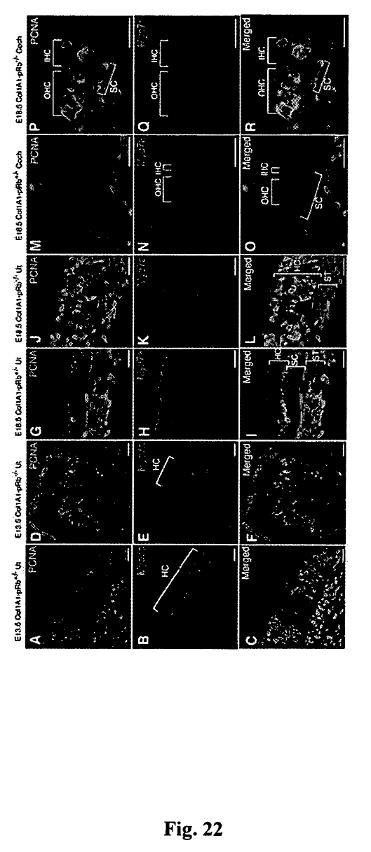

Dividing cells were also identified with an anti-PCNA antibody (A. L. Woods et al., Histopathology 19, 21 (1991)). In E13.5 and E18.5 Col1A1-pRb^{-/-} utricles but not in controls, most hair cells stained strongly for PCNA (FIG. 22). In cochleas as well, Col1A1-pRb^{-/-} hair cells and supporting cells were strongly PCNA-positive, unlike controls (FIG. 22, Panels P-R, and Panels M-O). Finally, hair cells in metaphase were observed in E18.5 Col1A1-pRb^{-/-} utricles (FIG. 20, Panel O). Staining with DAPI and an antibody to the hair-cell-specific transcription factor Brn-3.1 showed that, for hair cells in M-phase, Brn-3.1 labeling appeared to be cytoplasmic, and separated from DAPI-labeled condensed chromosomes that were segregating into two daughter nuclei during mitosis (FIG. 20, Panel O arrows and inset).

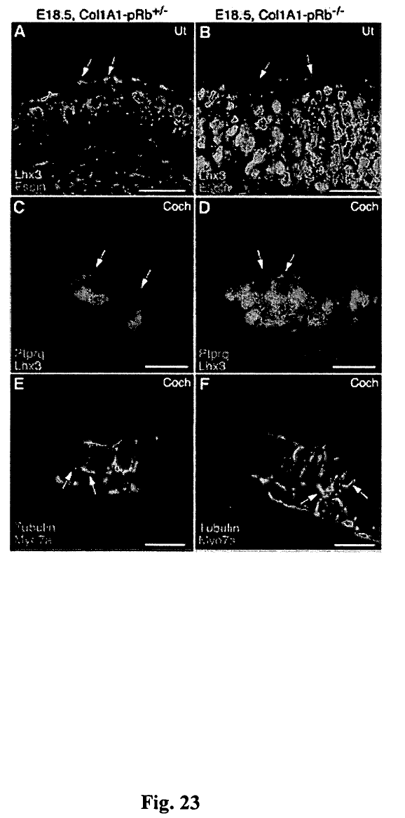

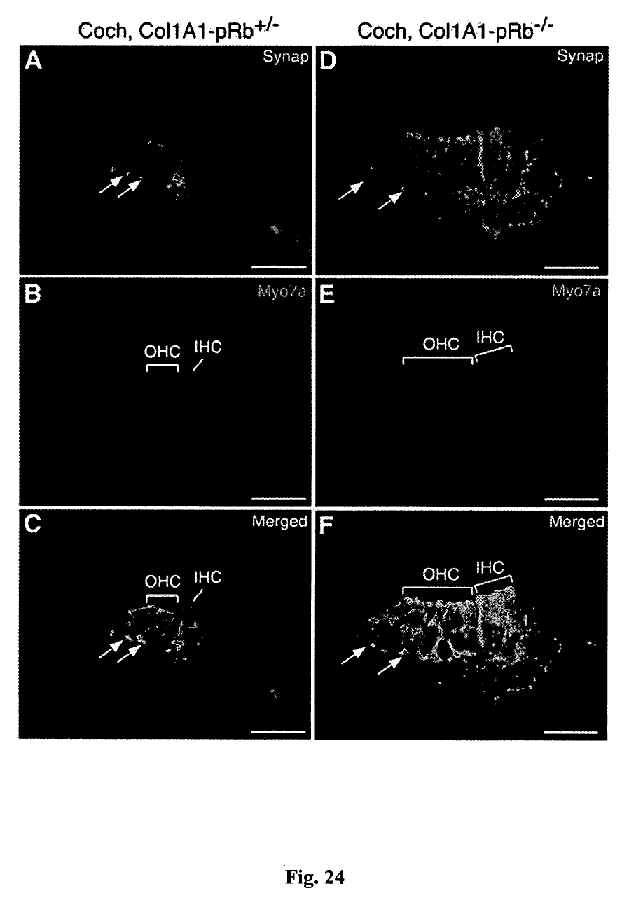

Most apical hair cells in E18.5 Col1A1-pRb^{-/-} utricles showed highly differentiated morphology, including pear-shaped cell bodies and intact hair bundles. Hair bundles were labeled with antibodies to espin (an actin crosslinker) and Ptprq (present in highly-differentiated cochlear hair bundles) (FIG. 23, Panels A and B, Panels C and D) (L. Zheng et al., Cell 102, 377 (2000); R. J. Goodyear et al., J Neurosci 23, 9208 (2003)). An antitubulin antibody revealed, as in controls, nerve fibers surrounding most Col1A1-pRb^{-/-} hair cells (FIG. 23, Panels E and F), and an antibody to the synaptic vesicle protein synaptophysin showed labeling around many Col1A1-pRb^{-/-} hair cells (FIG. 24), suggesting that Col1A1-pRb^{-/-} hair cells can attract axons and form synapses. Other markers of differentiated hair cells were also detected in Col1A1-pRb^{-/-} mice, including Brn-3.1 (FIG. 20, Panel O), Lhx3 (FIG. 23, Panels B and D), Gfil, Math1, calretinin and parvalbumin 3. In contrast to a conditional pRb-^{-/-} mouse model where retinal rods failed to differentiate (J. Zhang et al., Nat Genet 36, 351 (2004)), cell fate determination and subsequent differentiation were largely intact in the proliferating Col1A1-pRb^{-/-} hair cells. Therefore, Col1A1-pRb^{-/-} hair cells become differentiated without switching off proliferation, indicating that hair cell fate determination and differentiation do not require pRb function.

Loss of pRb does not eliminate hair cell function

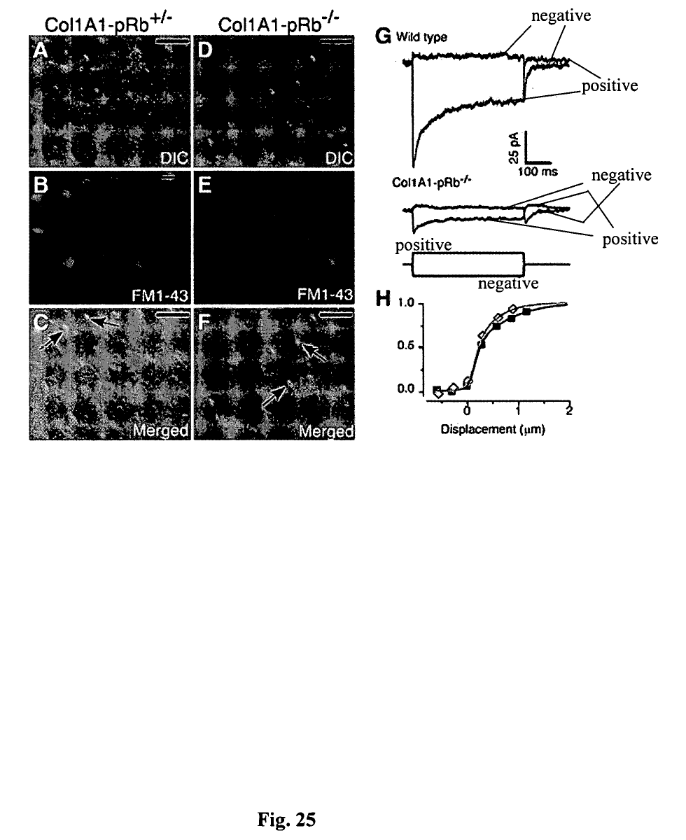

The sine qua non of hair cell function is mechanosensitivity. FM1-43, a fluorescent dye, enters hair cells through open mechanotransduction channels and so serves as a vital optical assay for mechanosensitivity (J. E. Gale, W. Marcotti, H. J. Kennedy, C. J. Kros, G. P. Richardson, J Neurosci 21, 7013 (2001); J. R. Meyers et al., J Neurosci 23, 4054 (2003)). FM1-43 labeling was observed in bundles and cell bodies of most hair cells in both control (FIG. 25, Panels A-C) and Col1A1-pRb^{-/-} utricles (FIG. 25, Panels D-F). Since most hair cells in Col1A1-pRb^{-/-} utricles are PCNA-positive, FM1-43 entry can occur in cycling hair cells. Transduction currents in control and Col1A1-pRb^{-/-} hair cells were recorded. Transduction currents were evoked in 4 randomly selected Col1A1-pRb^{-/-} hair cells (FIG. 25, Panels G and H), although currents were smaller than in controls (10-20 pA compared to ˜200 pA in controls). Currents might be smaller if bundles had little time to develop between cell divisions, especially with the known delay between bundle formation and transduction (G. S. Geleoc, J. R. Holt, Nat Neurosci 6, 1019 (2003)). Transduction currents showed a normal activation range and adaptation time course. Thus, specialized hair cell function does not require pRb.

Loss of pRb does not lead to apoptosis

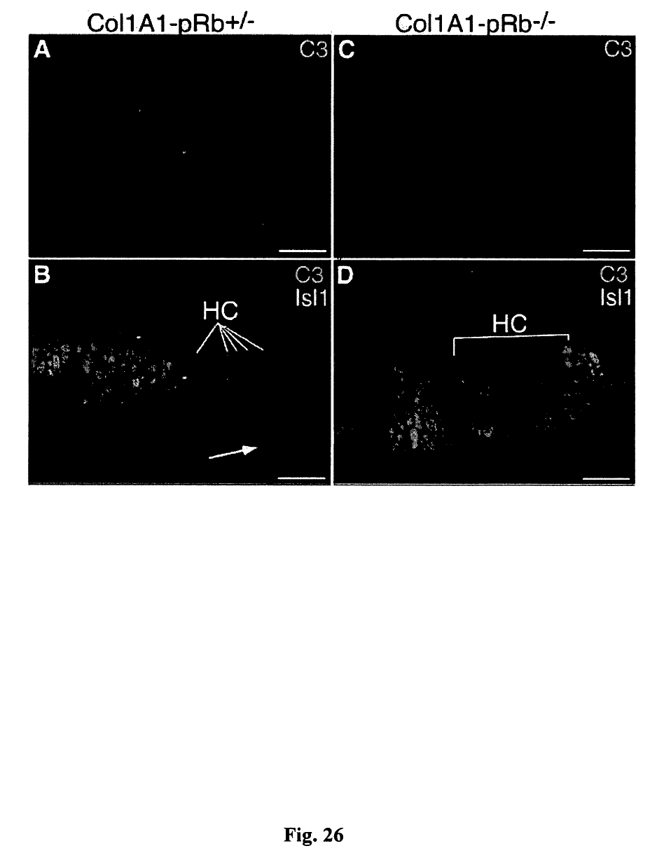

To determine whether apoptosis occurs in Col1A1-pRb^{-/-} hair cells, activated caspase-3 was assayed. Caspase-3-positive cells in Col1A1-pRb^{-/-} was not detected in sensory epithelium, nor in controls (FIG. 26). Therefore, loss of pRb itself does not appear to lead to cell death in the inner ear.

Evidence of postmitotic and mature hair cells can re-enter the cell cycle, after acute deletion of Rb1 gene

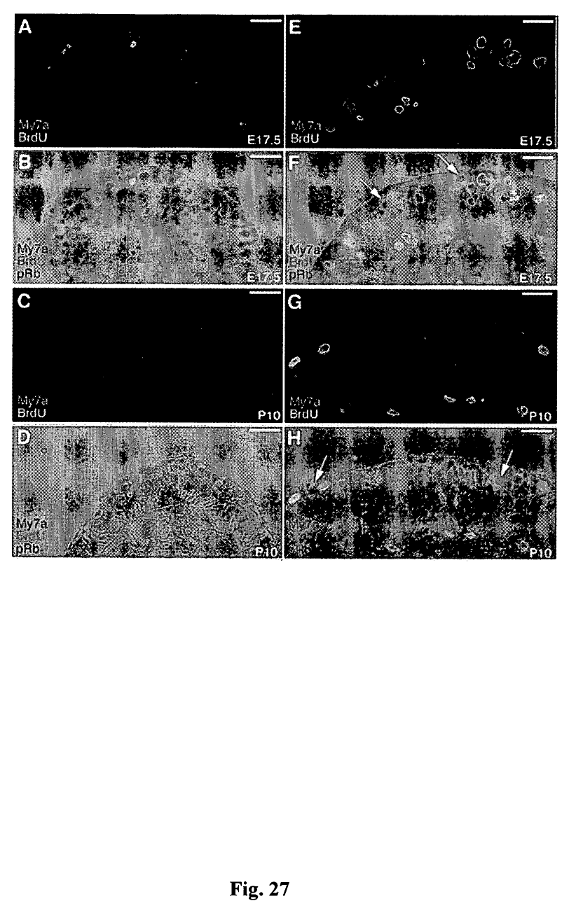

The prominent expression of Rb1 in postnatal hair cells and the fact that acute loss of pRb causes cell-cycle re-entry in quiescent or senescent cells ( J. Sage, A. L. Miller, P. A. Perez-Mancera, J. M. Wysocki, T. Jacks, Nature 424, 223 (Jul. 10, 2003)) are suggestive of a role for pRb in maintaining hair cells' non-proliferative status. To test this hypothesis, floxP-pRb utricles were cultured and infected with adenovirus carrying cre-recombinase, acutely deleting the Rb1 gene in infected hair cells (J. R. Holt et al., J Neurophysiol 81, 1881 (1999).) Utricular hair cells are mature at P10 and postmitotic at both stages studied (E17.5 and P10). After continuous culture in the presence of BrdU, no labeling was detected in adeno-GFP-infected (FIG. 27, Panels A-D) or uninfected floxP-pRb hair cells (FIG. 27, Panels F and H, arrows), whereas adeno-cre-recombinase infected hair cells incorporated BrdU (FIG. 27, Panels E-H). There were fewer BrdU labeled hair cells in P10 cultures than in E17.5 cultures, likely due to lower efficiency of infection of P10 hair cells. Additionally, more pRb was present in infected P10 hair cells following culture, suggesting that cre-mediated recombination or pRb degradation was less efficient in P10 cultures. All the infected hair cells lost hair bundles (J. R. Holt et al., J Neurophysiol 81, 1881 (1999)), so the function could not be tested. Nevertheless, the damaged hair cells re-entered the cell cycle.

Inner ear hair cells can continue to cell cycle in postnatal mice in vivo and are functional

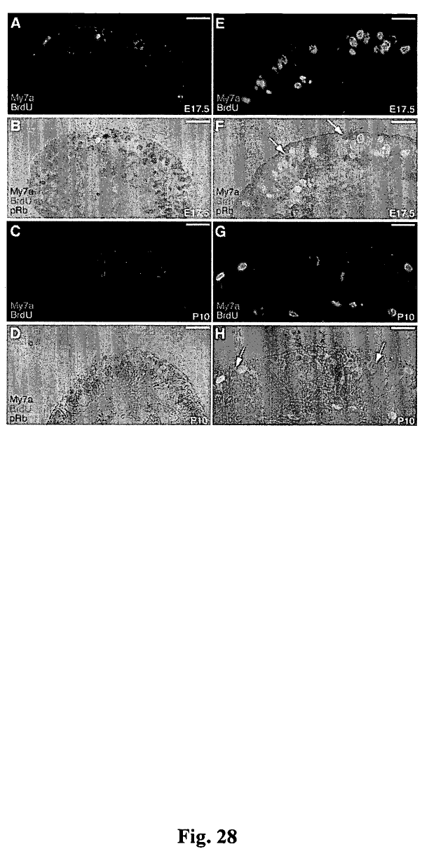

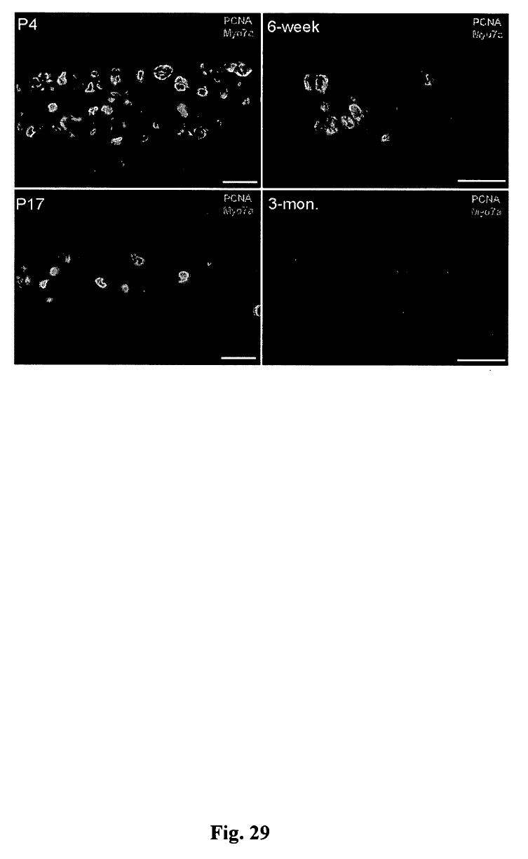

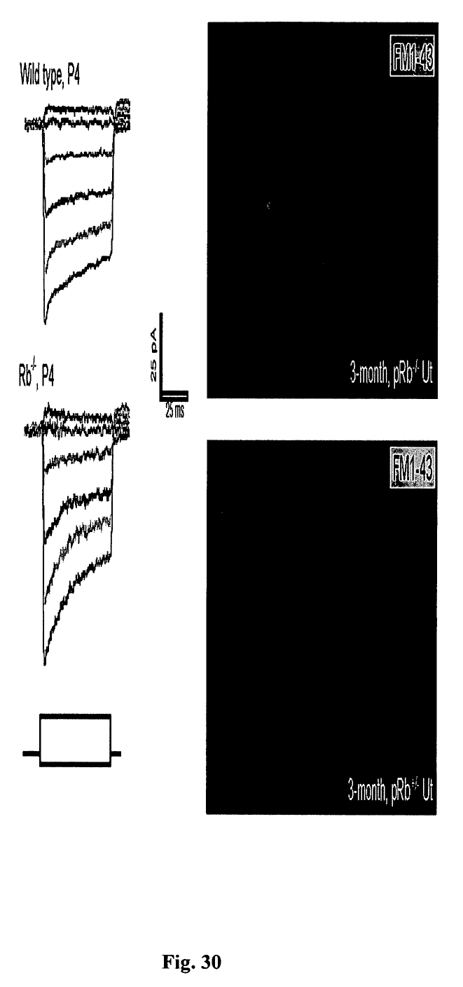

To test if proliferating hair cells can be maintained in the postnatal mice, a new pRb conditional knockout was created by breeding Brn-Cre and floxp-Rb mice. Brn-3.1 is a hair cell specific transcription factor. Brn-Cre-pRb mice survived postnatally because Brn-3.1 is only expressed in the hair cells and retinal ganglion cells. The hair cells of Brn-Cre-pRb mice were studied for their proliferation, hair bundle integrity and function. Hair cells in 3-month-old Brn-Cre-pRb mouse utricles were labeled with BrdU and PCNA (FIG. 28), indicating that they are proliferating hair cells. Confocal images of 6-month-old Brn-Cre-pRb utricular hair cells showed hair bundles. Moreover the experiment showed that 3- and 6-month-old Brn-Cre-pRb hair cells were capable of taking up FM1-43 through their signal transduction channels (FIG. 29). In addition, supporting cells in the Brn-Cre-pRb mice were induced to re-enter the cell cycle (FIG. 30), despite the fact that cre-recombinase is not expressed in supporting cells. Therefore, other signaling molecules were involved in the cell cycle re-entry of supporting cells.

pRb is also involved in cell cycle exit control of the sensory progenitor cells in the inner ear

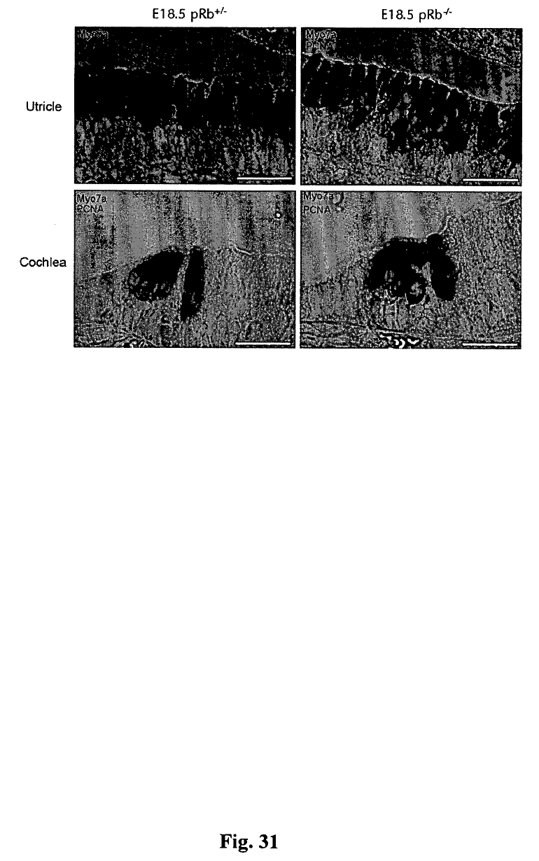

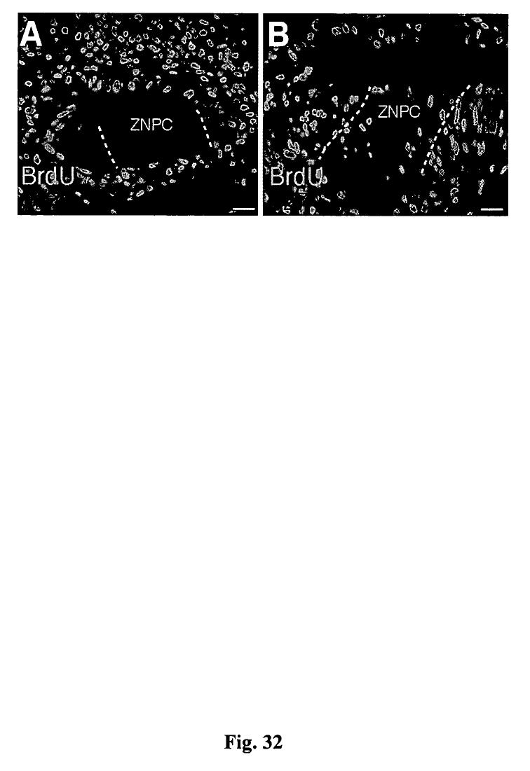

To study progenitor cell proliferation, E13.5 pregnant mice were injected with BrdU, 4 hours prior to embryo harvest. In the primordial organ of Corti, the p27Kip1-positive “zone of non-proliferating cells (ZPNC)” harbors postmitotic sensory precursor cells (P. Chen, J. E. Johnson, H. Y. Zoghbi, N. Segil, Development 129, 2495 (May, 2002).) BrdU-positive cells were found in the p27Kip1-positive region of Col1A1-pRb^{-/-} mice (FIG. 20, Panel N and FIG. 31, Panel B) but not in the controls (FIG. 20, Panel M and FIG. 31, Panel A). Therefore, pRb is involved in cell-cycle exit of sensory progenitor cells.

Supporting cell to hair cell conversion

Cochleas in Col1A1-pRb^{-/-} mice were studied for supporting-cell to hair-cell conversion. If this was the main pathway for increased hair cell number, it was expected p27Kip1-labeled supporting cells would label with Math1, the earliest hair cell marker; or Math1-positive cells would appear in supporting cell regions outside the hair-cell region. No p27Kip1 and Math1 double-positive cells, or Math1-positive cells in the supporting cell region were found. While cell fate conversion cannot be completely excluded, it is most likely that increased hair cell precursors and subsequent hair cell division are primarily responsible for the overproduction of hair cells.

From these studies it has been found that pRb regulates cell-cycle exit in hair cells, its loss permits cell-cycle re-entry and an increase hair cell numbers. These studies also demonstrate that differentiated mammalian hair cells can continue to cycle and divide in the absence of pRb, so that functional hair cells can be generated through divisions of preexisting hair cells. Furthermore, acute ablation of pRb in differentiated hair cells led to cell-cycle re-entry. Demonstration that pRb critically regulates hair cell division opens new opportunities, both for hair cell regeneration and for creating cell lines for hearing research. For hair cell regeneration, a reversible block of pRb function in hair cells could be used in place of permanent deletion of the Rb1 gene. Thus, regulated inactivation of pRb through use of, for example, siRNA, a small molecule inhibitor of pRb or reversible manipulation of pRb-modifying kinases could result in production of functional hair cells followed by restoration of normal cell-cycle exit.

These results also show that an irreversible switch from proliferation is not required for “terminal” differentiation, since cycling cells in the absence of pRb are highly differentiated and functional. These findings have implications for regenerating other functional cells (e.g., neuronal cells in the central nervous system (CNS), peripheral nervous system and inner ear) through manipulation of negative cell growth control genes.

### REFERENCES

Alexander, K., Hinds, P. W., Mol Cell Biol 21, 3616-31 (Jun. 2001).
Balsalobre, A. and Jolicoeur, P. (1995) Fos proteins can act as negative regulators of cell growth independently of the fos transforming pathway. Oncogene, 11, 455-465.
Bermingham, N. A., Hassan, B. A., Price, S. D., Vollrath, M. A., Ben-Arie, N., Eatock, R. A.,
Bellen, H. J., Lysakowski, A. and Zoghbi, H. Y. (1999) Math1: an essential gene for the generation of inner ear hair cells. Science, 284, 1837-1841.
Berns, A. (2003) Tumour suppressors: timing will tell. Nature, 424, 140-141.
Birren, S. J., Lo, L. and Anderson, D. J. (1993) Sympathetic neuroblasts undergo a developmental switch in trophic dependence. Development, 119, 597-610.
Bryant, J., Goodyear, R. J. and Richardson, G. P. (2002) Sensory organ development in the inner ear: molecular and cellular mechanisms. Br Med Bull, 63, 39-57.
Chan, H. M., Krstic-Demonacos, M., Smith, L., Demonacos, C. and La Thangue, N. B. (2001) Acetylation control of the retinoblastoma tumour-suppressor protein. Nat Cell Biol, 3, 667-674.
Chellappan, S. P., Hiebert, S., Mudryji, M., Horowitz, J. M., Nevins, J. R., Cell 65, 1053-61 (Jun. 14, 1991)
Chen, P., Johnson, J. E., Zoghbi, H. Y. and Segil, N. (2002) The role of Math1 in inner ear development: Uncoupling the establishment of the sensory primordium from hair cell fate determination. Development, 129, 2495-2505.
Chen, P. and Segil, N. (1999) p27(Kip1) links cell proliferation to morphogenesis in the developing organ of Corti. Development, 126, 1581-1590.
Chen, P., Zindy, F., Abdala, C., Liu, F., Li, X., Roussel, M. F. and Segil, N. (2003) Progressive hearing loss in mice lacking the cyclin-dependent kinase inhibitor Ink4d. Nat Cell Biol, 5, 422-426.
Chen, Z. Y. and Corey, D. P. (2002) An inner ear gene expression database. J Assoc Res Otolaryngol, 3, 140-148.
Classon, M. and Dyson, N. (2001) p107 and p130: versatile proteins with interesting pockets. Exp Cell Res, 264, 135-147.
Classon, M. and Harlow, E. (2002) The retinoblastoma tumour suppressor in development and cancer. Nat Rev Cancer, 2, 910-917.
Corey, D. P. and Hudspeth, A. J. (1980) Mechanical stimulation and micromanipulation with piezoelectric bimorph elements. J Neurosci Methods, 3, 183-202.
Corwin, J. T. (1998) Identifying the genes of hearing, deafness, and dysequilibrium. Proc Natl Acad Sci USA, 95, 12080-12082.
Corwin, J. T. and Cotanche, D. A. (1988) Regeneration of sensory hair cells after acoustic trauma. Science, 240, 1772-1774.
Dacquin, R., Starbuck, M., Schinke, T. and Karsenty, G. (2002) Mouse alpha1(I)-collagen promoter is the best known promoter to drive efficient Cre recombinase expression in osteoblast. Dev Dyn, 224, 245-251.
Deng, Y. et al., Genomics 63, 133-8 (Jan. 1, 2000)
Duan, M. L., Ulfendahl, M., Laurell, G., Counter, A. S., Pyykko, I., Borg, E. and Rosenhall, U. (2002) Protection and treatment of sensorineural hearing disorders caused by exogenous factors: experimental findings and potential clinical application. Hear Res, 169, 169-178.
Dyson, N. (1998) The regulation of E2F by pRB-family proteins. Genes Dev, 12, 2245-2262.
Dyson, N. and Harlow, E. (1992) Adenovirus E1A targets key regulators of cell proliferation. Cancer Surv, 12, 161-195.
Farah, M. H. et al., Development 127, 693-702 (February 2000)
Ferguson, K. L. and Slack, R. S. (2001) The Rb pathway in neurogenesis. Neuroreport, 12, A55-62.
Ferguson, K. L., Vanderluit, J. L., Hebert, J. M., McIntosh, W. C., Tibbo, E., MacLaurin, J. G., Park, D. S., Wallace, V. A., Vooijs, M., McConnell, S. K. and Slack, R. S. (2002) Telencephalon-specific Rb knockouts reveal enhanced neurogenesis, survival and abnormal cortical development. Embo J, 21, 3337-3346.
Forge, A., Li, L., Corwin, J. T. and Nevill, G. (1993) Ultrastructural evidence for hair cell regeneration in the mammalian inner ear. Science, 259, 1616-1619.
Gale, J. E., Marcotti, W., Kennedy, H. J., Kros, C. J. and Richardson, G. P. (2001) FM1-43 dye behaves as a permeant blocker of the hair-cell mechanotransducer channel. J Neurosci, 21, 7013-7025.
Geleoc, G. S. and Holt, J. R. (2003) Developmental acquisition of sensory transduction in hair cells of the mouse inner ear. Nat Neurosci, 6, 1019-1020.
Gloster, A., El-Bizri, H., Bamji, S. X., Rogers, D. and Miller, F. D. (1999) Early induction of Ta1pha1 alpha-tubulin transcription in neurons of the developing nervous system. J Comp Neurol, 405, 45-60.
Goodyear, R. J. et al., J. Neurosci., 23, 9208 (2003).
Holt, J. R., Corey, D. P. and Eatock, R. A. (1997) Mechanoelectrical transduction and adaptation in hair cells of the mouse utricle, a low-frequency vestibular organ. J Neurosci, 17, 8739-8748.
Holt, J. R. et al., J. Neurophysiol., 81, 1881 (1999).
Hu, Q. J., Dyson, N. and Harlow, E. (1990) The regions of the retinoblastoma protein needed for binding to adenovirus E1A or SV40 large T antigen are common sites for mutations. Embo J, 9, 1147-1155.
Hu, X. and Zuckerman, K. S. (2000) Cell cycle and transcriptional control of human myeloid leukemic cells by transforming growth factor beta. Leuk Lymphoma, 38, 235-246.
Irizarry, R. A., Hobbs, B., Collin, F., Beazer-Barclay, Y. D., Antonellis, K. J., Scherf, U. and Speed, T. P. (2003) Exploration, normalization, and summaries of high density oligonucleotide array probe level data. Biostatistics, 4, 249-264.
Jacks, T., Fazeli, A., Schmitt, E. M., Bronson, R. T., Goodell, M. A. and Weinberg, R. A. (1992) Effects of an Rb mutation in the mouse. Nature, 359, 295-300.
Jorgensen, J. M. and Mathiesen, C. (1988) The avian inner ear. Continuous production of hair cells in vestibular sensory organs, but not in the auditory papilla. Naturwissenschaften, 75, 319-320.
Kamiya, K., Takahashi, K., Kitamura, K., Momoi, T., Yoshikawa, Y., Brain Res 901, 296-302 (May 18, 2001).
Kawamoto, K., Ishimoto, S., Minoda, R., Brough, D. E. and Raphael, Y. (2003) Math1 gene transfer generates new cochlear hair cells in mature guinea pigs in vivo. J Neurosci, 23, 4395-4400.
Kiernan, A. E., Ahituv, N., Fuchs, H., Balling, R., Avraham, K. B., Steel, K. P. and Hrabe de Angelis, M. (2001) The Notch ligand Jagged1 is required for inner ear sensory development. Proc Natl Acad Sci USA, 98, 3873-3878.
Lanford, P. J., Lan, Y., Jiang, R., Lindsell, C., Weinmaster, G., Gridley, T. and Kelley, M. W. (1999) Notch signalling pathway mediates hair cell development in mammalian cochlea. Nat Genet, 21, 289-292.
Lasorella A. et al., Cancer Res 62, 301-6 (Jan. 1, 2002).
Lasorella, A., Noseda, M., Beyna, M. Yokota, Y., lavarone, A., Nature 407, 592-8 (Oct. 5, 2000)
Lauricella M., et al., FEBS Lett 499, 191-7 (Jun. 15, 2001).
Lipinski, M. M. and Jacks, T. (1999) The retinoblastoma gene family in differentiation and development. Oncogene, 18, 7873-7882.
Liu, F. et al., Int. J. Dev. Biol., 48, 645 (2004).
Lowenheim, H., Furness, D. N., Kil, J., Zinn, C., Gultig, K., Fero, M. L., Frost, D., Gummer, A. W., Roberts, J. M., Rubel, E. W., Hackney, C. M. and Zenner, H. P. (1999) Gene disruption of p27(Kip1) allows cell proliferation in the postnatal and adult organ of corti. Proc Natl Acad Sci USA, 96, 4084-4088.
Macleod, K. F., Hu, Y. and Jacks, T. (1996) Loss of Rb activates both p53-dependent and independent cell death pathways in the developing mouse nervous system. Embo J, 15, 6178-6188.
MacPherson, D., Sage, J., Crowley, D., Trumpp, A., Bronson, R. T. and Jacks, T. (2003) Conditional mutation of Rb causes cell cycle defects without apoptosis in the central nervous system. Mol Cell Biol, 23, 1044-1053.
Marino, S., Hoogervoorst, D., Brandner, S. and Berns, A. (2003) Rb and p107 are required for normal cerebellar development and granule cell survival but not for Purkinje cell persistence. Development, 130, 3359-3368.
Meyers, J. R., MacDonald, R. B., Duggan, A., Lenzi, D., Standaert, D. G., Corwin, J. T. and Corey, D. P. (2003) Lighting up the senses: FM1-43 loading of sensory cells through nonselective ion channels. J Neurosci, 23, 4054-4065.
Morris, E. J. and Dyson, N. J. (2001) Retinoblastoma protein partners. Adv Cancer Res, 82, 1-54.
Morton, C. C. (2002) Genetics, genomics and gene discovery in the auditory system. Hum Mol Genet, 11, 1229-1240.
Raz, Y. and Kelley, M. W. (1999) Retinoic acid signaling is necessary for the development of the organ of Corti. Dev Biol, 213, 180-193.
Ruben, R. T. (1967) Development of the inner ear of the mouse: a radioautographic study of terminal mitoses. Acta. Otolaryngol., 220, 4-44.
Rusch, A. and Eatock, R. A. (1996) Voltage responses of mouse utricular hair cells to injected currents. Ann N Y Acad Sci, 781, 71-84.
Rusch, A., Lysakowski, A., Eatock, R. A., J. Neurosci., 18, 7487 (1998).
Ryals, B. M. and Rubel, E. W. (1988) Hair cell regeneration after acoustic trauma in adult Coturnix quail. Science, 240, 1774-1776.
Sage, J., Miller, A. L., Perez-Mancera, P. A., Wysocki, J. M., Jacks, T., Nature 424, 223-8 (Jul. 10, 2003).
Schlingensiepen, K. H., Schlingensiepen, R., Kunst, M., Klinger, I., Gerdes, W., Seifert, W. and Brysch, W. (1993) Opposite functions of jun-B and c-jun in growth regulation and neuronal differentiation. Dev Genet, 14, 305-312.
Schacht, J., Ann N Y Acad Sci 884, 125-30 (Nov. 28, 1999)
Schreiber-Agus, N., Meng, Y., Hoang, T., Hou, H., Jr., Chen, K., Greenberg, R., Cordon-Cardo, C., Lee, H. W. and DePinho, R. A. (1998) Role of Mxi1 in ageing organ systems and the regulation of normal and neoplastic growth. Nature, 393, 483-487.
Self, T., Mahony, M., Fleming, J., Walsh, J., Brown, S. D. and Steel, K. P. (1998) Shaker-1 mutations reveal roles for myosin VIIA in both development and function of cochlear hair cells. Development, 125, 557-566.
Shim, J., et al., J. Biol Chem 275, 14107-11 (May 12, 2000).
Slack, R. S., El-Bizri, H., Wong, J., Belliveau, D. J. and Miller, F. D. (1998) A critical temporal requirement for the retinoblastoma protein family during neuronal determination. J Cell Biol, 140, 1497-1509.
Slack, R. S., Skerjanc, I. S., Lach, B., Craig, J., Jardine, K. and McBumey, M. W. (1995) Cells differentiating into neuroectoderm undergo apoptosis in the absence of functional retinoblastoma family proteins. J Cell Biol, 129, 779-788.
Thomas, D. M., Carty, S. A., Piscopo, D. M., Lee, J. S., Wang, W. F., Forrester, W. C. and Hinds, P. W. (2001) The retinoblastoma protein acts as a transcriptional coactivator required for osteogenic differentiation. Mol Cell, 8, 303-316.
Tiveron, M. C., Hirsch, M. R. and Brunet, J. F. (1996) The expression pattern of the transcription factor Phox2 delineates synaptic pathways of the autonomic nervous system. J Neurosci, 16, 7649-7660.
Vollrath, M. A., Eatock, R. A., J. Neurophysiol., 90, 2676 (2003).
Vooijs, M., te Riele, H., van der Valk, M., Berns, A, Oncogene, 21, 4635 (2002).
Wang, A., Liang, Y., Fridell, R. A., Probst, F. J., Wilcox, E. R., Touchman, J. W., Morton, C. C., Morell, R. J., Noben-Trauth, K., Camper, S. A. and Friedman, T. B. (1998) Association of unconventional myosin MYO15 mutations with human nonsyndromic deafness DFNB3 [see comments]. Science, 280, 1447-1451.
Wang, J. et al., J. Neurosci 23, 8596-607 (Sept. 17, 2003).
Warchol, M. E., Lambert, P. R., Goldstein, B. J., Forge, A. and Corwin, J. T. (1993) Regenerative proliferation in inner ear sensory epithelia from adult guinea pigs and humans. Science, 259, 1619-1622.
Woods, A. L., Hall, P. A., Shepherd, N. A., Hanby, A. M., Waseem, N. H., Lane, D. P. and Levison, D. A. (1991) The assessment of proliferating cell nuclear antigen (PCNA) immunostaining in primary gastrointestinal lymphomas and its relationship to histological grade, S+G2+M phase fraction (flow cytometric analysis) and prognosis. Histopathology, 19, 21-27.
Wu, W. J., Sha, S. H., Schacht, J. Audiol Neurootol 7, 171-4 (May-June, 2002).
Xiang, M., Gao, W. Q., Hasson, T., Shin, J. J., Development, 125, 3935 (1998).
Yoshikawa, K. (2000) Cell cycle regulators in neural stem cells and postmitotic neurons. Neurosci Res, 37, 1-14.
Zhang, J. et al., Nat. Genet., 36, 351 (2004).
Zhang, N., Martin, G. V., Kelley, M. W. and Gridley, T. (2000) A mutation in the Lunatic fringe gene suppresses the effects of a Jagged2 mutation on inner hair cell development in the cochlea. Curr Biol, 10, 659-662.
Zheng, J. L. and Gao, W. Q. (2000) Overexpression of Math1 induces robust production of extra hair cells in postnatal rat inner ears. Nat Neurosci, 3, 580-586.
Zheng, L. et al., Cell, 102, 377 (2000).
Zine, A. and de Ribaupierre, F. (2002) Notch/Notch ligands and Math1 expression patterns in the organ of Corti of wild-type and Hes1 and Hes5 mutant mice. Hear Res, 170, 22-31.
Zine, A., Van De Water, T. R. and de Ribaupierre, F. (2000) Notch signaling regulates the pattern of auditory hair cell differentiation in mammals. Development, 127, 3373-3383.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

We claim:

1. A method for generating functional, differentiated inner ear hair cells, comprising:
eliminating or reducing the expression level or function of pRb in inner ear sensory cells by an amount effective to generate functional, differentiated inner ear hair cells.

2. The method of claim 1, wherein the inner ear sensory cells are progenitor cells.

3. The method of claim 1, wherein the inner ear sensory cells are supporting cells.

4.-6. (canceled)

7. The method of claim 1, wherein the inner ear sensory cells are hair cells.

8.-12. (canceled)

13. The method of claim 1, wherein the expression level or function is reduced or eliminated with a RNAi or siRNA molecule.

14.-21. (canceled)

22. A method for restoring hearing or balance to a subject, comprising:
eliminating or reducing the expression level or function of pRb in the inner ear sensory cells of the subject by an amount effective to generate functional, differentiated inner ear hair cells to restore hearing or balance to the subject.

23. The method of claim 22, wherein the subject suffers from hearing damage due to a viral infection, noise, a mutation in a gene which causes hair cell death, or ototoxic drug exposure.

24.-28. (canceled)

29. The method of claim 22, wherein the inner ear sensory cells are hair cells.

30.-34. (canceled)

35. The method of claim 22, wherein the expression level or function is reduced or eliminated with a RNAi or siRNA molecule.

36.-42. (canceled)

43. A method for restoring hearing or balance to a subject, comprising:
providing to the subject in need thereof functional, differentiated inner ear hair cells generated by the elimination or reduction of the expression level or function of pRb in inner ear sensory epithelial cells.

44.-48. (canceled)

49. The method of claim 43, wherein the inner ear sensory cells are hair cells.

50.-53. (canceled)

54. The method of claim 43, wherein the expression level or function is reduced or eliminated with an antisense oligonucleotide, a RNAi or siRNA molecule or an intrabody.

55. (canceled)

56. A functional, differentiated inner ear hair cell line.

57. The functional, differentiated inner ear hair cell line of claim 56, wherein the functional, differentiated inner ear hair cell line is composed of functional, differentiated inner ear hair cells with reduced or eliminated expression level or function of pRb.

58. (canceled)

59. The functional, differentiated inner ear hair cell line of claim 57, wherein the expression level or function is reduced or eliminated with an antisense oligonucleotide, a RNAi or siRNA molecule or an intrabody.

60.-61. (canceled)

62. An inner ear sensory epithelial cell or cell line, wherein the expression level or function of pRb is eliminated or reduced.

63.-64. (canceled)

65. The inner ear sensory epithelial cell line of claim 62, wherein the expression level or function is reduced or eliminated with an antisense oligonucleotide, a RNAi or siRNA molecule or an intrabody.

66.-76. (canceled)

77. A screening method for identifying compounds for regenerating or protecting hair cells, comprising:
contacting a candidate compound with a sample containing cells of a functional, differentiated inner ear hair cell line, and
determining if the candidate compound affects the production of or protects the functional, differentiated inner ear hair cells.

78. The method of claim 77, wherein the cells have reduced or eliminated pRb expression level or function.

79.-94. (canceled)

95. A vector or plasmid, comprising:
a hair cell-specific promoter and a nucleic acid that is hybridizable to a retinoblastoma gene or a transcript thereof or a complement thereof.

96.-100. (canceled)
